(19) United States
(12) Patent Application Publication
(10) Pub. No.: US 2014/0296343 A1
(43) Pub. Date: Oct. 2, 2014

(54) NON-PEPTIDE BDNF NEUROTROPHIN MIMETICS

(71) Applicant: PharmatrophiX, Inc., Menlo Park, CA (US)

(72) Inventors: Frank M. LONGO, Menlo Park, CA (US); Stephen M. MASSA, Burlingame, CA (US); Sandeep GUPTA, Concord, OH (US)

(21) Appl. No.: 14/230,821

(22) Filed: Mar. 31, 2014

Related U.S. Application Data
(63) Continuation of application No. 13/117,537, filed on May 27, 2011, now abandoned.
(60) Provisional application No. 61/349,770, filed on May 28, 2010.

(51) Int. Cl. C07C 33/30; C07C 215/68; C07C 33/26; C07C 43/205; C07D 401/10; C07C 237/34; C07D 211/42; C07D 295/135; C07D 215/20; C07C 327/48; C07C 237/42

(52) U.S. Cl. CPC C07C 33/30; C07C 327/48; C07C 215/68; C07C 33/26; C07C 43/2055; C07C 237/42; C07C 237/34; C07D 211/42; C07D 295/135; C07D 215/20; C07D 401/10
USPC 514/599; 564/74; 564/315; 568/811; 514/730; 568/648; 514/718; 564/153; 514/616; 564/157; 514/617; 564/156; 546/216; 544/165; 546/153; 546/188; 435/375

(57) ABSTRACT

Methods and compounds for treating neurological and other disorders are provided. Included is the administering to a subject in need thereof an effective amount of a compound having binding and/or modulation specificity for a TrkB receptor molecule.

### CROSS-REFERENCE TO RELATED APPLICATION

The application claims the benefit of priority of U.S. Provisional Application No. 61/349,770, filed on May 28, 2011 and entitled “Non-Peptide BDNF Neurotrophin Mimetics”, the contents of which are hereby incorporated by reference in their entireties for all purposes.

### TECHNICAL FIELD

The presently disclosed subject matter generally relates to the treatment of disorders in a subject, including but not limited to neurological disorders. More particularly, the methods of the presently disclosed subject matter relate to administering to a subject an effective amount of a compound having binding and/or modulation specificity for the TrkB receptor molecule to treat a disorder in the subject.

### BACKGROUND

Neurotrophins are polypeptides that play a role in the development, function, and/or survival of certain cells, including neurons. The death or dysfunction of neurons has been directly implicated in a number of neurological disorders. It has been suggested that alterations in neurotrophin localization, expression levels of neurotrophins, and/or expression levels of the receptors that bind neurotrophins are linked to neuronal degeneration or dysfunction. This degeneration or dysfunction can occur in the neurological disorders Alzheimer's, Parkinson's, Huntington's disease and amyotrophic lateral sclerosis (ALS), among others. Neurotrophins also mediate fundamental mechanisms relevant to non-neurological disorders including for example depression, obesity, and ischemic conditions of peripheral tissues.

A variety of neurotrophins have been identified, including Nerve Growth Factor (NGF), Neurotrophin-3 (NT-3), Neurotrophin-4/5 (NT-4/5), Neurotrophin 6 (NT-6) and Brain Derived Neurotrophic Factor (BDNF). Neurotrophins are found in both precursor form, known as pro-neurotrophins, and in mature form. The mature forms are proteins of about 120 amino acids in length that exist in physiological states as stable, non-covalent approximately 25 kDa homodimers. Each neurotrophin monomer includes three solvent-exposed .beta.-hairpin loops, referred to as loops 1, 2, and 4 that exhibit relatively high degrees of amino acid conservation across the neurotrophin family.

Mature neurotrophins bind preferentially to the receptors Trk and p75^{NTR}, while pro-neurotrophins, which contain an N-terminal domain proteolytically removed in mature forms, interact principally with the p75^{NTR} receptor and through their N-terminal domains, with the sorting receptor sortilin (Fahnestock, M., Michalski, B., Xu, B., Coughlin M. D. (2001) Mol Cell Neurosci 18, 210-220; Harrington, A. W. et al. (2004) Proc Natl Acad Sci USA 101, 6226-6230; Nykjaer, A. et al., (2004) Nature 427, 843-848). The p75^{NTR} receptor interacts with Trks and modulates Trk signaling, but is also independently coupled to several signaling systems, including pro-survival signals, IRAK/TRAF6/NF.kappa.B, PI3/AKT, and pro-apoptotic signals, NRAGE/JNK (Mamidipudi, V., Li, X., Wooten, M. W. (2002) J Biol Chem 277, 28010-28018; Roux, P. P., Bhakar. A. L., Kennedy, T. E., Barker, P. A. (2001) J Biol Chem 276, 23097-23104; Salehi, A. H., et al. (2000) Neuron 27, 279-288).

Depending on the operative ligands, co-expression of Trk or other receptors, and expression of downstream signaling elements p75^{NTR} promotes cell survival or death. proNGF induces death of superior cervical ganglion neurons and oligodendrocytes through p75^{NTR}, and its comitant binding to p75^{NTR} and sortilin has been shown to activate cell death pathways (Nykjaer, A. et al., (2004) Nature 427, 843-848; Lee, R., Kermani, P., Teng, K. K., Hempstead, B. L. (2001) Science 294, 1945-1948; Beattie, M. S., et al. (2002) Neuron 36, 375-386).

When administered for therapeutic use, neurotrophins exhibit suboptimal pharmacological properties, including poor stability with low serum half lives, likely poor oral bioavailability, and restricted central nervous system penetration (Podulso, J. F., Curran, G. L. (1996) Brain Res Mol Brain Res 36, 280-286; Saltzman, W. M., Mak, M. W., Mahoney, M. J., Duenas, E. T., Cleland, J. L. (1999) Pharm Res 16, 232-240; Partridge, W. M. (2002) Adv Exp Med Bio 513, 397-430). Additionally, the highly pleiotropic effects of neurotrophins achieved through action of the triple receptor signaling network increases the chances of adverse effects.

Unfortunately, technical and ethical considerations have thus far hampered the development of therapeutic agents based upon neurotrophins. For example, it is technically difficult to produce sufficient quantities of pure neurotrophins using recombinant DNA techniques. Additionally, although it is possible to utilize human fetal cells to produce neurotrophins, the ethical ramifications raised by the use of such cells (typically obtained from an aborted fetus) have all but prevented the utilization of this approach.

Previous studies have described the creation of synthetic peptides corresponding to various domains of the BDNF protein that are capable of achieving the BDNF effect of promoting neurite outgrowth (O'Leary and Hughes, 2003; Williams et al., 2005; Fletcher and Hughes, 2006). While it is not known if these synthetic BDNF peptides actually activate the TrkB receptor or whether they achieve their neurotrophic effects by a non-TrkB mechanism, these peptides are too large (approximately 2000 MW) to constitute actual medicinal compounds.

Accordingly, there is an unmet need in the art for the development of small molecule (for example, <500 MW, characteristic of successful drugs) non-peptidyl or peptide agents based upon neurotrophins for use in the treatment of disorders. In particular, there is a need to identify small molecules that mimic key regions of neurotrophin proteins and have the ability to activate the TrkB receptor, optionally in combination with a TrkA or TrkC receptor. There is further a need for small molecules that target TrkB receptors optionally in combination with TrkA or TrkC receptors to avoid or minimize potentially deleterious interactions with the p75^{NTR} and sortilin receptors.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

Disclosed herein are compounds having binding and/or modulation specificity for a TrkB receptor molecule, optionally in combination with a TrkA or TrkC receptor molecule.

Also disclosed herein are methods of treating a disorder in a subject, including both neurological and non-neurological disorders, comprising administering to the subject an effective amount of a small molecule compound of the invention.

In some embodiments, the disorder is selected from the group consisting of Alzheimer's disease, Huntington's disease, amyotrophic lateral sclerosis, Rett syndrome, epilepsy, Parkinson's disease, spinal cord injury, stroke, hypoxia, ischemia, brain injury, diabetic neuropathy, peripheral neuropathy, nerve transplantation complications, motor neuron disease, multiple sclerosis, HIV dementia, peripheral nerve injury, hearing loss, depression, obesity, metabolic syndrome, pain, cancer, and conditions involving degeneration or dysfunction of cells expressing TrkB.

Also disclosed herein are methods of facilitating neural cell survival or promoting neural function comprising treating a neural cell with a compound of the invention having the ability to specifically bind and/or modulate the activity of a TrkB receptor molecule, optionally in combination with TrkA or TrkC receptor molecule.

### DETAILED DESCRIPTION

In subjects with particular disorders, including neurological and other disorders, alterations in neurotrophin localization, expression levels of neurotrophins, and/or expression levels of the receptors that bind neurotrophins can occur. Accordingly, by providing subjects suffering from such disorders with a corresponding neurotrophic factor or mimetic thereof, such neural degeneration can be alleviated or prevented. In some cases, inhibition of neurotrophin function would be of benefit. As disclosed for the first time herein, methods of treating a disorder and/or facilitating neural cell survival by administering a non-peptide compound having binding and/or modulation specificity for the TrkB receptor molecule are provided.

### DEFINITIONS

It is to be understood that the terminology used herein is for the purpose of describing particular embodiments only and is not intended to be limiting.

Throughout the specification and claims, a given chemical formula or name shall encompass all optical and stereoisomers, as well as racemic mixtures where such isomers and mixtures exist.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application. Generally the term “about”, as used herein when referring to a measurable value such as an amount of weight, time, dose, etc. is meant to encompass in one example variations of ±20% or ±10%, in another example ±5%, in another example ±1%, and in yet another example ±0.1% from the specified amount, as such variations are appropriate to perform the disclosed method.

As used herein, the term “neurological disorder” includes any disorder characterized by damage of nervous system cells and include the following, without limitation, Alzheimer's disease, Huntington's disease, amyotrophic lateral sclerosis (ALS), epilepsy, Parkinson's disease, spinal cord injury, stroke, hypoxia, ischemia, brain injury, diabetic neuropathy, peripheral neuropathy, nerve transplantation complications, multiple sclerosis, peripheral nerve injury, and conditions involving degeneration or dysfunction of cells expressing Trkb.

The term “alkylene,” alone or in combination, refers to an optionally substituted straight-chain or branched-chain alkyl radical having from 1 to about 20 carbon atoms. The term also includes optionally substituted straight-chain or branched-chain alkyl radicals having from 1 to about 6 carbon atoms as well as those having from 1 to about 4 carbon atoms. Examples of alkyl radicals include methyl, ethyl, n-propyl, isopropyl, n-butyl, isobutyl, sec-butyl, tert-butyl, tert-amyl, pentyl, hexyl, heptyl, octyl and the like. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls. Alkyl groups can be optionally substituted.

The term “heteroalkyl” refers to alkyl groups, as described above, in which one or more skeletal atoms are oxygen, nitrogen, sulfur or combinations thereof. The term heteroalkyl also includes alkyl groups in which one 1 to about 6 skeletal atoms are oxygen, nitrogen, sulfur or combinations thereof, as well as those in which 1 to 4 skeletal atoms are oxygen, nitrogen, sulfur or combinations thereof and those in which 1 to 2 skeletal atoms are oxygen, nitrogen, sulfur or combinations thereof. Heteroalkyl groups are optionally substituted.

The term “alkenyl,” alone or in combination, refers to an optionally substituted straight-chain or branched-chain hydrocarbon radical having one or more carbon-carbon double-bonds and having from 2 to about 18 carbon atoms. The term also includes optionally substituted straight-chain or branched-chain hydrocarbon radicals having one or more carbon-carbon double bonds and having from 2 to about 6 carbon atoms as well as those having from 2 to about 4 carbon atoms. Examples of alkenyl radicals include ethenyl, propenyl, butenyl, 1,4-butadienyl and the like. Suitable alkenyl groups include allyl. The terms “allylic group” or “allyl” refer to the group —CH₂HC═CH₂ and derivatives thereof formed by substitution. Thus, the terms alkenyl and/or substituted alkenyl include allyl groups, such as but not limited to, allyl, methylallyl, di-methylallyl, and the like. The term “allylic position” or “allylic site” refers to the saturated carbon atom of an allylic group. Thus, a group, such as a hydroxyl group or other substituent group, attached at an allylic site can be referred to as “allylic.” “1-alkenyl” refers to alkenyl groups where the double bond is between the first and second carbon atom.

The term “alkynyl,” alone or in combination, refers to an optionally substituted straight-chain or branched-chain hydrocarbon radical having one or more carbon-carbon triple-bonds and having from 2 to about 12 carbon atoms. The term also includes optionally substituted straight-chain or branched-chain hydrocarbon radicals having one or more carbon-carbon triple bonds and having from 2 to about 6 carbon atoms as well as those having from 2 to about 4 carbon atoms. Examples of alkynyl radicals include ethynyl, propynyl, butynyl and the like. “1-alkynyl” refers to alkynyl groups where the triple bond is between the first and second carbon atom.

“Cyclic alkyl” and “cycloalkyl” refer to a non-aromatic mono- or multicyclic ring system of about 3 to about 10 carbon atoms, e.g., 3, 4, 5, 6, 7, 8, 9, or 10 carbon atoms, alternately from about 3 to about 6 carbon atoms. The cycloalkyl group can be optionally partially unsaturated, such as for example cyclohexadiene, e.g. cyclohexa-1,4-diene. The cycloalkyl group also can be optionally substituted as defined herein. Representative monocyclic cycloalkyl rings include, but are not limited to, cyclopropyl, cyclobutyl, cyclopentyl, cyclohexyl, cycloheptyl, and the like. Further, the cycloalkyl group can be optionally substituted with a linking group, such as an alkylene group as defined hereinabove, for example, methylene, ethylene, propylene, and the like. In such cases, the cycloalkyl group can be referred to as, for example, cyclopropylmethyl, cyclobutylmethyl, and the like. Additionally, multicyclic cycloalkyl rings include adamantyl, octahydronaphthyl, decalin, camphor, camphane, and noradamantyl.

The term “heterocyclic alkyl” and “heterocycloalkyl” refer to cyclic groups of 3 to 6 atoms, containing at least one heteroatom. In one aspect, these groups contain 1 to 3 heteroatoms. Suitable heteroatoms include oxygen, sulfur, and nitrogen. Heterocyclic groups may be attached through a nitrogen or through a carbon atom in the ring. Suitable heterocyclic groups include pyrrolidinyl, morpholino, imidazolidinyl, pyrazolidinyl, piperidyl, piperazyl, dithianyl, dioxanyl, thiomorpholinyl, tetrahydrofuranyl, and pyridyl. Such groups may be substituted.

The term “aryl” refers to aromatic groups which have 5-14 ring atoms and at least one ring having a conjugated pi electron system and includes carbocyclic aryl, heterocyclic aryl and biaryl groups, all of which may be optionally substituted. The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The aromatic ring(s) can comprise phenyl, naphthyl, biphenyl, diphenylether, diphenylamine and benzophenone, among others. all of which can be optionally substituted. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings. Examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like, all optionally substituted.

“Carbocyclic aryl” groups are groups wherein the ring atoms on the aromatic ring are carbon atoms. Carbocyclic aryl groups include monocyclic carbocyclic aryl groups and polycyclic or fused compounds such as optionally substituted naphthyl groups.

“Heterocyclic aryl” or “heteroaryl” groups are groups containing at least one aromatic ring and having from 1 to 4 heteroatoms as ring atoms with the remainder of the ring atoms being carbon atoms. Heteroaryl and heterocyclic aryl include both monocyclic and bicyclic ring systems. Such groups may be substituted. Suitable heteroatoms include oxygen, sulfur, nitrogen, and selenium. Suitable monocyclic heteroaryl groups include furanyl, thienyl, pyridyl, pyrrolyl, N-lower alkyl pyrrolyl, pyridyl-N-oxide, pyrimidyl, pyrazinyl, imidazolyl, and the like, all optionally substituted. Suitable bicyclic heteroaryl groups include quinolinyl, tetrahydroquinolinyl, isoquinolinyl, tetrahydroisoquinolinyl, indolyl, isoindolyl, indolinyl, benzimidazolyl, benzopyrrolyl, benzoxazolyl, benzothiazolyl, oxazolopyridinyl, thiazolopyridinyl, imidazolopyridinyl, benzofuranyl, benzothiophenyl, indazolyl, quinazolinyl, quinoxalinyl and phthalazinyl.

The phrase “carbocyclic ring” refers to a saturated or unsaturated monocyclic or bicyclic ring in which all atoms of all rings are carbon. Thus, the term includes cycloalkyl and carbocyclic aryl rings.

The phrase “heterocyclic ring” refers to a saturated or unsaturated monocyclic or bicyclic ring having from 1 to 4 heteroatoms as ring atoms in the aromatic ring and the remainder of the ring atoms being carbon atoms. Thus, the term includes heterocycloalkyl and heterocyclic aryl rings.

The term “optionally substituted” or “substituted” includes groups substituted by one to four substituents, independently selected from lower alkyl, lower aryl, lower aralkyl, lower alicyclic, heterocyclic alkyl, hydroxyl, lower alkoxy, lower aryloxy, perhaloalkoxy, aralkoxy, heteroaryl, heteroaryloxy, heteroarylalkyl, heteroaralkoxy, azido, amino, guanidino, amidino, halo, lower alkylthio, oxo, acylalkyl, carboxy esters, carboxyl, -carboxamido, nitro, acyloxy, aminoalkyl, alkylaminoaryl, alkylaryl, alkylaminoalkyl, alkoxyaryl, arylamino, aralkylamino, phosphono, sulfonyl, -carboxamidoalkylaryl, -carboxamidoaryl, hydroxyalkyl, haloalkyl, alkylaminoalkylcarboxy-, aminocarboxamidoalkyl-, cyano, lower alkoxyalkyl, lower perhaloalkyl, and arylalkyloxyalkyl.

When a named atom of a ring or chain is defined as being “absent,” the named atom is replaced by a direct bond or is incorporated into double bond along with the atom to which it is attached. When the linking group or spacer group is defined as being absent, the linking group or spacer group is replaced by a direct bond.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Alkoxyl” or “alkoxyalkyl” refer to an alkyl-O— group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl- group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Dialkylamino” refers to an —NRR′ group wherein each of R and R′ is independently an alkyl group and/or a substituted alkyl group as previously described. Exemplary alkylamino groups include ethylmethylamino, dimethylamino, and diethylamino.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is alkyl and/or substituted alkyl as previously described.

“Dialkylcarbamoyl” refers to a R′RN—CO— group wherein each of R and R′ is independently alkyl and/or substituted alkyl as previously described.

“Acyloxyl” refers to an acyl-O— group wherein acyl is as previously described.

“Acylamino” refers to an acyl-NH— group wherein acyl is as previously described.

“Acylamino” refers to an aroyl-NH— group wherein aroyl is as previously described.

The term “amino” refers to the —NH₂ group.

The term “carbonyl” refers to the —(C═O)— group.

The term “carboxyl” refers to the —COOH group.

The term “cyano” refers to the —CN group.

The terms “halo”, “halide”, or “halogen” as used herein refer to fluoro, chloro, bromo, and iodo groups.

The term “hydroxyl” refers to the —OH group.

The term “hydroxyalkylene” refers to an alkylene group substituted with an —OH group; hydroxyalkenyl refers to an alkenyl group substituted with an —OH group; hydroxyalkynyl refers to an alkynyl group substituted with an —OH group.

The term “aminoalkylene” refers to an alkylene group substituted with an —NH₂ group; aminoalkenyl refers to an alkenyl group substituted with an —NH₂ group; aminoyalkynyl refers to an alkynyl group substituted with an —NH₂ group.

The term “mercapto” refers to the —SH group.

The term “oxo” refers to ═O.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term “cycloalkenyl” refers to a partially unsaturated cyclic hydrocarbon group containing one or more rings, for example, one ring, two rings, three rings, or four rings, with three or more carbon atoms per ring, for example, 3, 4, 5, 6, 7, or 8 carbon atoms per ring. Exemplary cycloalkenyl groups include, but are not limited to, cyclopropenyl, cyclobutenyl, cyclopentenyl, cyclohexenyl, and the like. Cycloalkenyl groups can be optionally substituted, such as with one or more substituents, e.g. 1, 2, 3, or 4 substituents, at any available point of attachment. Exemplary substituents include, but are not limited to, alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl.

When the term “independently selected” is used, the substituents being referred to (e.g., R groups, such as groups R¹ and R², or groups X and Y), can be identical or different. For example, both R¹ and R² can be substituted alkyls, or R¹ can be hydrogen and R² can be a substituted alkyl, and the like.

The term “treatment” as used herein covers any treatment of a disease and/or condition in an animal or mammal, particularly a human, and includes: (i) preventing a disease, disorder and/or condition from occurring in a person which can be predisposed to the disease, disorder and/or condition, or at risk for being exposed to an agent that can cause the disease, disorder, and/or condition; but, has not yet been diagnosed as having it; (ii) inhibiting the disease, disorder and/or condition, i.e., arresting its development; and (iii) relieving the disease, disorder and/or condition, i.e., causing regression of the disease, disorder and/or condition.

“Binding specificity” refers to the ability of a protein or other type of molecule capable of recognizing and interacting with a complementary site on another protein or other type of molecule. As used herein, the term binding specificity can refer to the ability of a molecule to bind preferentially to one type of molecule over another. For example, binding specificity for TrkB can refer to the ability of a BDNF mimetic to preferentially bind to TrkB. In one embodiment binding specificity for TrkB can refer to the ability of a BDNF mimetic to preferentially bind to TrkB and TrkC as opposed to other receptors or proteins; in another embodiment, binding specificity for TrkB can refer to the ability of a BDNF mimetic to preferentially bind to TrkB and TrkA as opposed to other receptors or protein. A molecule having binding specificity for a receptor can be used for one or more of contacting the receptor, activating the receptor, and inhibiting the receptor.

The term “modulation specificity” as used herein refers to a molecule that can modulate the activity of one receptor preferentially. The molecule can modulate the activity of one receptor to a greater extent than another receptor or can modulate the activity of one receptor in a group of receptors exclusively. For example, a BDNF mimetic can specifically modulate the activity of TrkB. Modulation specificity for TrkB can refer to the ability of a BDNF mimetic to preferentially modulate TrkB. In one embodiment modulation specificity for TrkB can refer to the ability of a BDNF mimetic to preferentially modulate TrkB and TrkC as opposed to other receptors or proteins; in another embodiment, modulation specificity for TrkB can refer to the ability of a BDNF mimetic to preferentially modulate TrkB and TrkA as opposed to other receptors or protein. The modulation of activity can include, but is not limited to, upregulation, downregulation, activation, partial activation, agonism, partial agonism, antagonism, partial antagonism, inhibition, partial inhibition, or a combination thereof. A molecule having modulation specificity for a receptor can be used, for example, to contact and activate a receptor or to contact and inhibit a receptor.

The term “binding and/or modulation specificity” refers to a molecule that can bind a designated receptor, modulate the activity of a designated receptor, or both bind and modulate the activity of a designated receptor.

The term “pharmacophore”, as used herein, refers to a specific model or representation of a molecular moiety capable of exerting a selected biochemical effect, e.g., inhibition of an enzyme, binding to a receptor, chelation of an ion, and the like. A selected pharmacophore can have more than one biochemical effect, e.g., can be an inhibitor of one enzyme and an agonist of a second enzyme. A therapeutic agent can include one or more pharmacophores, which can have the same or different biochemical activities.

The term “derivative” as used herein refers to a compound chemically modified so as to differentiate it from a parent compound. Such chemical modifications can include, for example, replacement of hydrogen by an alkyl, acyl, or amino group. A derivative compound can be modified by, for example, glycosylation, pegylation, or any similar process that retains at least one biological or immunological function of the compound from which it was derived.

The term “stereoisomer” as it relates to a given compound is well understood in the art, and refers to another compound having the same molecular formula, wherein the atoms making up the other compound differ in the way they are oriented in space, but wherein the atoms in the other compound are like the atoms in the given compound with respect to which atoms are joined to which other atoms (e.g., an enantiomer, a diastereomer, or a geometric isomer).

The term “hydrophilicity” is used in the common manner of the field as having an affinity for water; readily absorbing and/or dissolving in water.

The term “lipophilicity” is used in the common manner of the field as having an affinity for, tending to combine with, or capable of dissolving in lipids.

The term “amphipathicity”, as used herein, describes a structure having discrete hydrophobic and hydrophilic regions. Thus, one portion of the structure interacts favorably with aqueous and other polar media, while another portion of the structure interacts favorably with non-polar media.

The term “solubility” as used herein, describes the maximum amount of solute that will dissolve in a given amount of solvent at a specified temperature.

The term “bioavailability” as used herein refers to the systemic availability (i.e., blood/plasma levels) of a given amount of compound administered to a subject. The term further encompasses the rate and extent of absorption of compound that reaches the site of action.

Tautomers of the compounds of the invention are encompassed by the present application. Thus, for example, a carbonyl includes its hydroxyl tautomer.

As used herein “solvate” refers to a complex of variable stoichiometry formed by a solute (e.g. a compound of formula (I) or (II) or a salt, ester or prodrug thereof) and a solvent. Such solvents for the purpose of the invention may not interfere with the biological activity of the solute. Examples of suitable solvents include water, methanol, ethanol and acetic acid. Generally the solvent used is a pharmaceutically acceptable solvent. Examples of suitable pharmaceutically acceptable solvents include water, ethanol and acetic acid. Generally the solvent used is water.

The present invention further relates to an ester of the compounds of the invention, for example an in vivo hydrolysable ester. An in vivo hydrolysable ester of a compound which contains carboxy or hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid.

The present invention includes prodrugs of the compounds of the invention. In general, such prodrugs will be functional derivatives of these compounds that are readily convertible in vivo into the required compound of the invention. Conventional procedures for the selection and preparation of suitable prodrug derivatives are described, for example, in “Design of Prodrugs”, ed. H. Bundgaard, Elsevier, 1985. Such prodrugs include but are not limited to ester prodrugs from alcohols and acids as well as phosphate prodrugs of alcohols, all of which are familiar to those of skill in the art. The prodrug can be formulated to achieve a goal of improved chemical stability, improved patient acceptance and compliance, improved bioavailability, prolonged duration of action, improved organ selectivity, improved formulation (e.g., increased hydrosolubility), and/or decreased side effects (e.g., toxicity).

Table of Abbreviations

2D—two-dimensional
3D—three-dimensional
Aβ—amyloid-β
Ab—antibody
AD—Alzheimer's disease
ALS—amyotrophic lateral sclerosis
BCA—bicinchoninic acid
BDNF—brain-derived neurotrophic factor
b.i.d.—twice daily
cm—centimeter
d—day
D—Dalton
DMEM—Dulbecco's Modified Eagle Media
ECL—electrogenerated chemiluminescence
EDTA—ethylenediamine tetraacetic acid
ELISA—Enzyme Linked ImmunoSorbent Assay
ERK—extracellular signal-regulated protein kinase
FBS—fetal bovine serum
g—gram
h—hour
HBA—hydrogen bond acceptor
HBD—hydrogen bond donor
HEPES—4-2-hydroxyethyl-1-piperazineethanesulfonic acid
HRP—horseradish peroxidase
IgG—Immunoglobin G
IP—Intraperitoneal
IV—intravenous
K³²—lysine residue number 32
kcal—kilocalorie
kg—kilogram
MBP—myelin basic protein
mg—milligram
min—minute
ml—milliliter
mM—millimolar
mol—mole
MTT—3-(4,5-dimethylthiazol-2-yl)-2,5-diphenyltetrazolium bromide
MW—molecular weight
NaCl—sodium chloride
ng—nanogram
nM—nanomolar
NS—not significant
NMR—nuclear magnetic resonance
NGF—nerve growth factor
nM—nanomolar
p—probability
p75^{NTR}—p75 neurotrophin receptor
PBS—phosphate-buffered saline
pmol—picomole
PMSF—phenylmethylsulfonyl fluoride
PO—per os (by mouth)
pro-NGF—unprocessed precursor of NGF
PVDF—Polyvinylidine Difluoride
SDS—sodium dodecyl sulfate
SE—standard error
s.e.m.—standard error of measurement
Tris—2-Amino-2-(hydroxymethyl)-1,3-propanediol
TUNEL—Terminal deoxynucleotidyl transferase-mediated deoxyuridine
triphosphate nick-end labeling
μg—microgram
μl—microliter
μM—micromolar
%—percent
° C.—degrees Celsius
≧—greater than or equal to
>—greater than
≦—less than or equal to
<—less than

### Embodiments of Compounds

The presently disclosed subject matter provides compounds having binding and/or modulation specificity for the TrkB receptor molecule. In some embodiments, the compounds bind to and/or modulate both TrkB and TrkA; in other embodiments, the compounds of the invention bind to and/or modulate both TrkB and TrkC. The compounds may be mimetics of BDNF, in some embodiments, specifically mimetics of the β-turn loop 2 of BDNF. The compounds of the invention can be used in accordance with the presently disclosed pharmaceutical compounds and methods in the treatment and prevention of disorders, including but not limited to neurological disorders (e.g., neurodegenerative disorders).

Some TrkB binding and/or modulation compounds demonstrate agonist function and thus promote TrkB activation. Some TrkB binding and/or modulation compounds demonstrate partial agonist function. These compounds can be used to promote TrkB function or in some cases to partially block the function of endogenous BDNF. Inhibition of BDNF function can prove useful for prevention or treatment of epilepsy or other disorders in which excessive BDNF function contributes to underlying disease mechanisms. Some TrkB binding and/or modulation compounds demonstrate no agonist activity and thus might prove useful as TrkB antagonists.

The compounds of the presently disclosed subject matter can be isolated from natural sources, purchased from commercial sources, or synthesized or partially synthesized by methodology known in the art of synthetic organic chemistry, including parallel and combinatorial synthetic techniques.

In accordance with one aspect of the presently disclosed subject matter, a representative compound or mimetic of BDNF β-turn loop 2 having binding and/or modulation specificity for a TrkB receptor molecule can comprise a compound having a structure of Formula (I) or Formula (II), each as defined herein.

In one aspect, the present application discloses a compound of Formula (I):

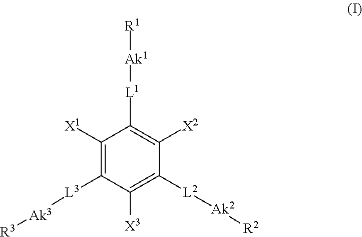

wherein X¹, X², and X³ each independently is —H, halo or C₁-C₆ alkyl; Ak¹, Ak², and Ak³ each independently is optionally substituted C₁-C₆ alkylene, optionally substituted C₂-C₆ alkenyl, or optionally substituted C₂-C₆ alkynyl; L¹, L², and L³ each independently is —O—, —S—, —NH—, —C(O)NH—, —C(S)NH—, —CR_{A}R_{B}CR_{A′}R_{B′}— or —CR_{A}═CR_{A′}—; or alternatively, one or two of -L¹-Ak¹-R¹, -L²-Ak²—R², and -L³-Ak³-R³ is —H; R¹, R², and R³ each independently is —OH, —NH₂, —OR^{A}, —NR^{A}R^{B}, an optionally substituted heterocycloalkyl or an optionally substituted heteroaryl; and each of R^{A}, R^{B}, R^{A′}, and R^{B′} independently is —H, halo, or C₁-C₆ alkyl; or a pharmaceutically acceptable salt, ester, prodrug, or solvate thereof.

In one embodiment, the compound does not have the formula:

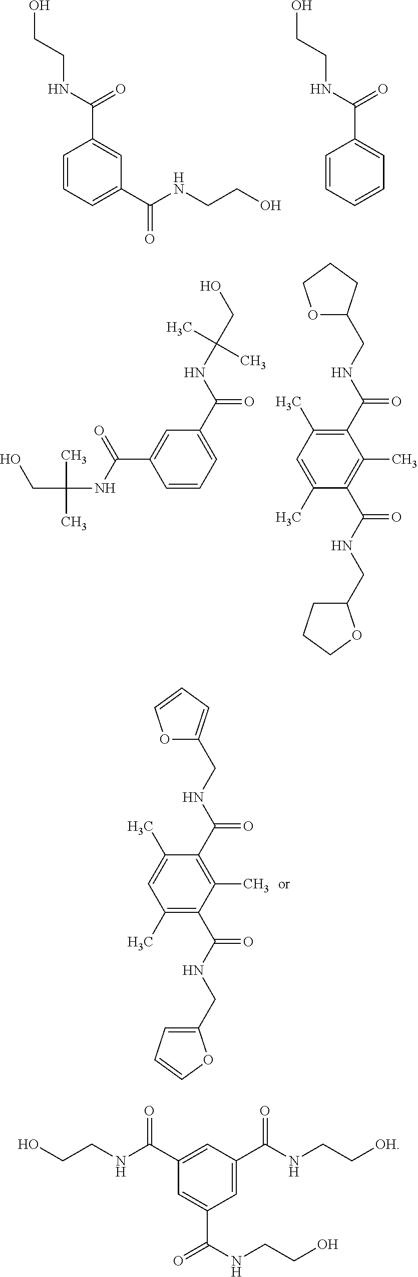

In one embodiment the present application discloses a compound of Formula (I) wherein each of X¹, X², and X³ is —H, —F, —Cl or —CH₃; Ak¹, Ak², and Ak³ each independently is optionally substituted C₁-C₆ alkylene; R¹, R², and R³ each independently is —OH, —NH₂, —OR^{A}, —NR^{A}R^{B}, optionally substituted heterocycloalkyl or optionally substituted monocyclic heteroaryl; and each of R^{A}, R^{B}, R^{A′}, and R^{B′} independently is —H, —F, —Cl, —CH₂, or —CH₂—CH₃; or a pharmaceutically acceptable salt, ester, prodrug, or solvate thereof. In another embodiment, each of X¹, X², and X³ is —H; Ak¹, Ak², and Ak³ each independently is optionally substituted C₁-C₄ alkylene; and R¹, R², and R³ each independently is —OH, —NH₂, —OR^{A}, or —NR^{A}R^{B}; or a pharmaceutically acceptable salt, ester, prodrug, or solvate thereof. In another embodiment, Ak¹, Ak², and Ak³ each independently is optionally substituted C₂-C₃ alkylene; and L¹, L², and L³ each independently is —O—, —C(O)NH—, —C(S)NH—, —CH₂CH₂— or —CH═CH—; or a pharmaceutically acceptable salt, ester, prodrug, or solvate thereof.

In one embodiment the present application discloses a compound of Formula (I) wherein at least one of X¹, X², and X³ is —Cl; Ak¹, Ak², and Ak³ each independently is optionally substituted C₁-C₄ alkylene; R¹, R², and R³ each independently is —OH, —NH₂, —OR^{A}, or —NR^{A}R^{B}; and each of R^{A}, R^{B}, R^{A′}, and R^{B′} each independently is —H, —F, or —Cl; or a pharmaceutically acceptable salt, ester, prodrug, or solvate thereof. In another embodiment, at least two of X¹, X², and X³ is —Cl; Ak¹, Ak², and Ak³ each is —CH₂—CH₂—; L¹, L², and L³ each independently is —O—, —S—, —NH—, —C(O)NH—, —C(S)NH—, —CH₂CH₂— or —CH═CH—; and R¹, R², and R³ each independently is —OH or —NH₂; or a pharmaceutically acceptable salt, ester, prodrug, or solvate thereof.

In one embodiment the present application discloses a compound having a structural formula selected from the group consisting of:

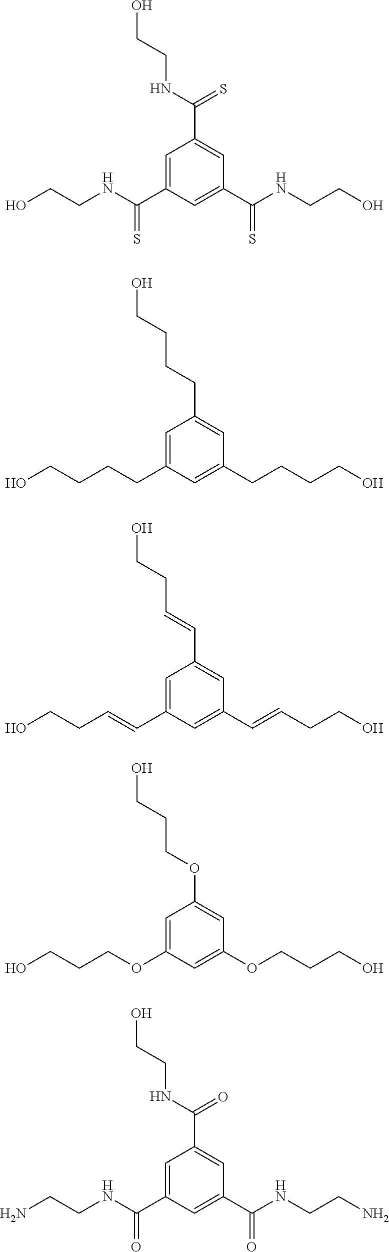

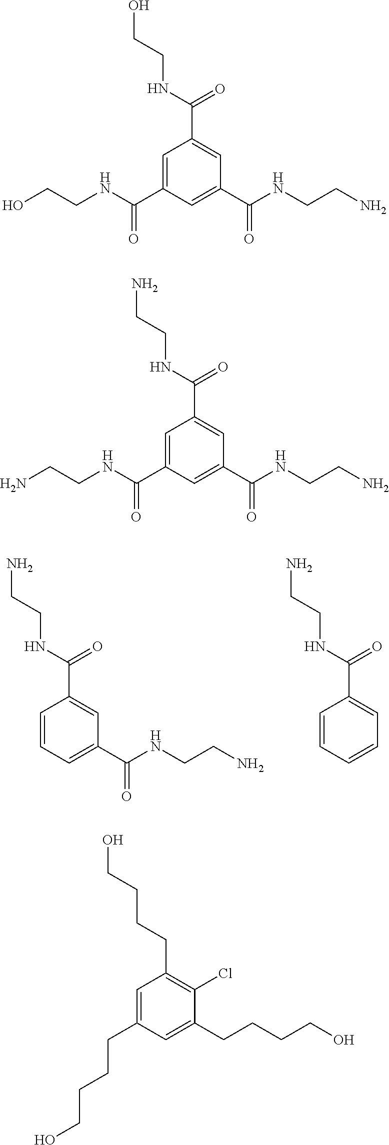

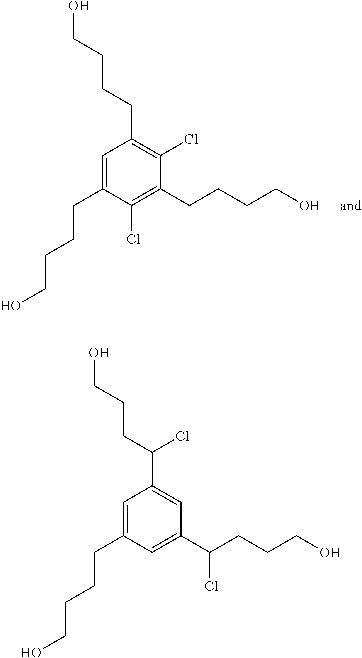 [SMILES: OCCCCc1cc(CCCCO)c(Cl)c(CCCCO)c1Cl ; OCCCCc1cc(C(Cl)CCCO)cc(C(Cl)CCCO)c1]

or a pharmaceutically acceptable salt, ester, prodrug, or solvate thereof.

In one aspect, the present application discloses a compound of Formula (I):

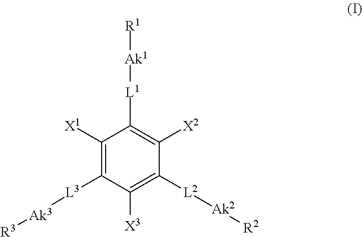

wherein X¹, X², and X³ each independently is —H or halo; Ak¹, Ak², and Ak³ each independently is optionally substituted C₁-C₆ alkylene, optionally substituted C₂-C₆ alkenyl, or optionally substituted C₂-C₆ alkynyl; L¹, L², and L³ each independently is —O—, —S—, —NH—, —C(O)NH—, —C(S)NH—, —CR^{A}R^{B}CR^{A′}R^{B′}— or —CR^{A}═CR^{A′}—; or alternatively, one or two of -L¹-Ak¹-R¹, -L²-Ak²-R², and -L³-Ak³-R³ is —H; R¹, R², and R³ each independently is —OH, —NH₂₅—OR^{A}, or —NR^{A}R^{B}; and each of R^{A}, R^{B}, R^{A′}, and R^{B′} independently is —H, halo, or C₁-C₆ alkyl; or a pharmaceutically acceptable salt, ester, prodrug, or solvate thereof.

In one embodiment, the compound does not have the formula:

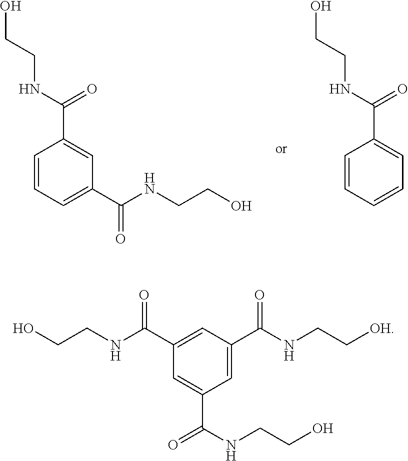 [SMILES: O=C(NCCO)c1cccc(C(=O)NCCO)c1 ; O=C(NCCO)c1ccccc1 ; O=C(NCCO)c1cc(C(=O)NCCO)cc(C(=O)NCCO)c1]

In another embodiment, L¹, L², and L³ each independently is —O—, —S—, —NH—, —C(O)NH—, —C(S)NH—, —CR^{A}R^{B}CR^{A′}R^{B′}— or —CR^{A}═CR^{A′}—; or alternatively, one of -L¹-Ak¹-R¹, -L²-Ak²-R², and -L³-Ak³-R³ is —H; alternately, L¹, L², and L³ each independently is —O—, —S—, —NH—, —C(O)NH—, —C(S)NH—, —CR^{A}R^{B}, CR^{A′}R^{B′}— or —CR^{A}═CR^{A′}—. In one embodiment Ak¹, Ak², and Ak³ each independently is optionally substituted C₁-C₆ alkylene. In one embodiment, each of X¹, X², and X³ is —H, —F, or —Cl; Ak¹, Ak², and Ak³ each independently is optionally substituted C₁-C₆ alkylene; each of R^{A}, R^{B}, R^{A′}, and R^{B′} independently is —H, —F, or —Cl; or a pharmaceutically acceptable salt, ester, prodrug, or solvate thereof. In another embodiment, each of X¹, X², and X³ is —H; Ak¹, Ak², and Ak³ each independently is optionally substituted C₁-C₄ alkylene; and R¹, R², and R³ each independently is —OH or —NH₂; or a pharmaceutically acceptable salt, ester, prodrug, or solvate thereof. In another embodiment, each of X¹, X², and X³ is —H; Ak¹, Ak², and Ak³ each independently is optionally substituted C₂-C₃ alkylene; and L¹, L², and L³ each independently is —O—, —C(O)NH—, —C(S)NH—, —CH₂CH₂— or —CH═CH—; R¹, R², and R³ each independently is —OH or —NH₂; or a pharmaceutically acceptable salt, ester, prodrug, or solvate thereof. In yet another embodiment, at least one of X¹, X², and X³ is —Cl; Ak¹, Ak², and Ak³ each independently is optionally substituted C₁-C₄ alkylene; R¹, R², and R³ each independently is —OH, —NH₂, —OR^{A}, or —NR^{A}R^{B}; and each of R^{A}, R^{B}, R^{A′}, and R^{B′} each independently is —H, —F, or —Cl; or a pharmaceutically acceptable salt, ester, prodrug, or solvate thereof. In one variation, at least two of X¹, X², and X³ is —Cl; Ak¹, Ak², and Ak³ each is —CH₂—CH₂—; L¹, L², and L³ each independently is —O—, —S—, —NH—, —C(O)NH—, —C(S)NH—, —CH₂CH₂— or —CH═CH—; and R¹, R², and R³ each independently is —OH or —NH₂; or a pharmaceutically acceptable salt, ester, prodrug, or solvate thereof.

In one aspect, the present application discloses a compound of Formula (II):

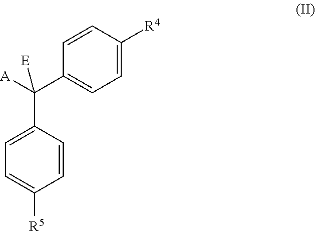

wherein: E is —H; each of R⁴ and R⁵ is independently halo, —NR^{C}R^{D}, optionally substituted heterocycloalkyl; or optionally substituted phenyl; A is —H, C₁-C₆ alkylene, C₂-C₆ alkenyl, C₂-C₆ alkynyl, optionally substituted aryl, or optionally substituted heteroaryl; and each of R^{C} and R^{D} is independently —H, C₁-C₆ alkylene, C₁-C₆ aminoalkylene, C₂-C₆ aminoalkenyl, C₂-C₆ aminoalkynyl, C₁-C₆ hydroxyalkylene, C₂-C₆ hydroxyalkenyl, C₂-C₆ hydroxyalkynyl; or a pharmaceutically acceptable salt, ester, prodrug, or solvate thereof.

In one embodiment, the compound does not have the formula:

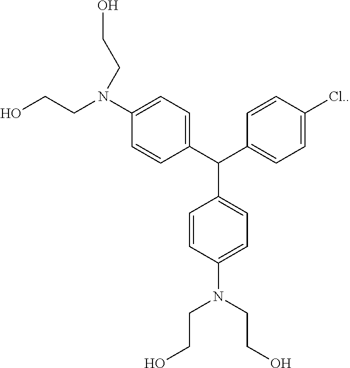 [SMILES: OCCN(CCO)c1ccc(C(c2ccc(Cl)cc2)c2ccc(N(CCO)CCO)cc2)cc1]

In one embodiment the present application discloses a compound of Formula (II) wherein each of R⁴ and R⁵ is independently —F, —Cl, —NR^{C}R^{D}, optionally substituted morpholinyl, optionally substituted thiomorpholinyl, optionally substituted piperazinyl, optionally substituted piperidinyl, optionally substituted pyrrolidinyl, or phenol; A is —H, C₁-C₆ alkylene, optionally substituted phenyl, or optionally substituted bicyclic heteroaryl; and each of R^{C} and R^{D} is independently —H, C₁-C₆ alkylene, C₁-C₆ aminoalkylene, or C₁-C₆ hydroxyalkylene; or a pharmaceutically acceptable salt, ester, prodrug, or solvate thereof. In another embodiment, each of R⁴ and R⁵ is independently —F, —Cl, —NR^{C}R^{D}, optionally substituted N-bound morpholinyl, optionally substituted N-bound piperidinyl, or phenol; A is —H, C₃-C₆ alkylene, optionally substituted phenyl, optionally substituted quinolinyl, optionally substituted tetrahydroquinolinyl, optionally substituted indolinyl; and each of R^{C} and R^{D} is independently —H, methyl, ethyl, C₂-C₄ aminoalkylene, or C₂-C₄ hydroxyalkylene; or a pharmaceutically acceptable salt, ester, prodrug, or solvate thereof. In one variation, A is —H or a pharmaceutically acceptable salt, ester, prodrug, or solvate thereof. In another variation, A is C₃-C₆ alkylene or a pharmaceutically acceptable salt, ester, prodrug, or solvate thereof. In yet another variation, A is quinolinyl substituted with one or more of —OH and C₁-C₆ hydroxyalkylene, or a pharmaceutically acceptable salt, ester, prodrug, or solvate thereof; alternately, A is quinolinyl substituted with C₂-C₄ hydroxyalkylene, or a pharmaceutically acceptable salt, ester, prodrug, or solvate thereof. In still a further variation, A is isoquinolinyl substituted with one or more of —OH and C₁-C₆ hydroxyalkylene, or a pharmaceutically acceptable salt, ester, prodrug, or solvate thereof; alternately, A is isoquinolinyl substituted with C₂-C₄ hydroxyalkylene, or a pharmaceutically acceptable salt, ester, prodrug, or solvate thereof. In yet another variation, A is optionally substituted phenyl, or a pharmaceutically acceptable salt, ester, prodrug, or solvate thereof; alternately, A is para-substituted phenyl, wherein the para-substituent is —Cl or —NR^{C}R^{D}, wherein each of R^{C} and R^{D} is independently —H, C₁-C₆ aminoalkylene, or C₁-C₆ hydroxyalkylene; or a pharmaceutically acceptable salt, ester, prodrug, or solvate thereof. In some embodiments, the para-substituent is —NR^{C}R^{D}, wherein each of R^{C} and R^{D} is C₂-C₄ aminoalkylene, or C₂-C₄ hydroxyalkylene; or a pharmaceutically acceptable salt, ester, prodrug, or solvate thereof. In one variation each of R^{C} and R^{D} is —CH₂CH₂—OH or a pharmaceutically acceptable salt, ester, prodrug, or solvate thereof.

In one aspect, the present application discloses a compound having a structural formula selected from the group consisting of:

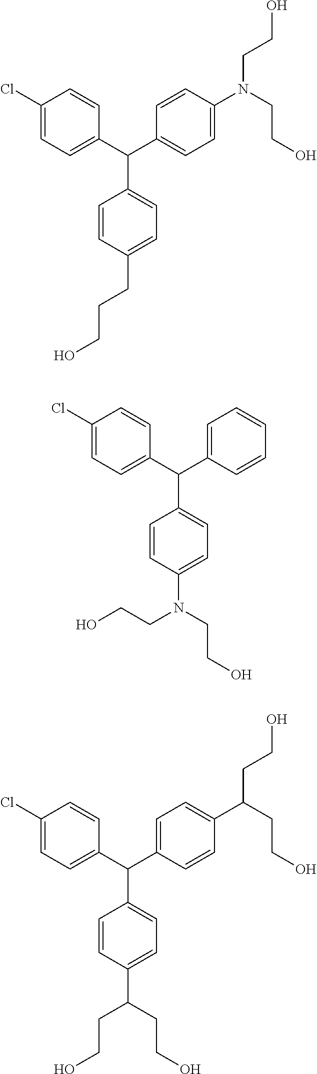 [SMILES: OCCCc1ccc(C(c2ccc(Cl)cc2)c2ccc(N(CCO)CCO)cc2)cc1 ; OCCN(CCO)c1ccc(C(c2ccccc2)c2ccc(Cl)cc2)cc1 ; OCCC(CCO)c1ccc(C(c2ccc(Cl)cc2)c2ccc(C(CCO)CCO)cc2)cc1]

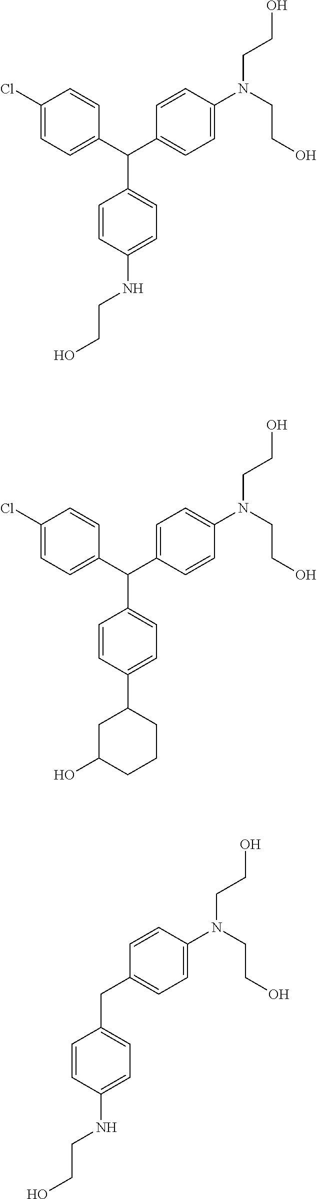 [SMILES: OCCNc1ccc(C(c2ccc(Cl)cc2)c2ccc(N(CCO)CCO)cc2)cc1 ; OCCN(CCO)c1ccc(C(c2ccc(Cl)cc2)c2ccc(C3CCCC(O)C3)cc2)cc1 ; OCCNc1ccc(Cc2ccc(N(CCO)CCO)cc2)cc1]

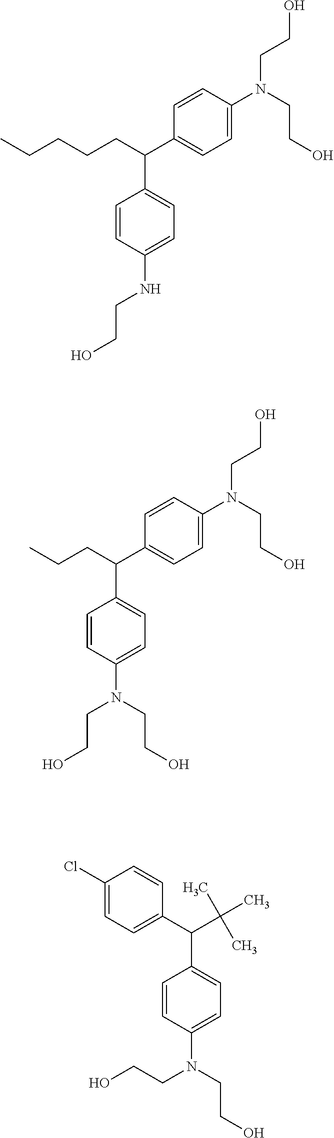 [SMILES: CCCCCC(c1ccc(NCCO)cc1)c1ccc(N(CCO)CCO)cc1 ; CCCC(c1ccc(N(CCO)CCO)cc1)c1ccc(N(CCO)CCO)cc1 ; CC(C)(C)C(c1ccc(Cl)cc1)c1ccc(N(CCO)CCO)cc1]

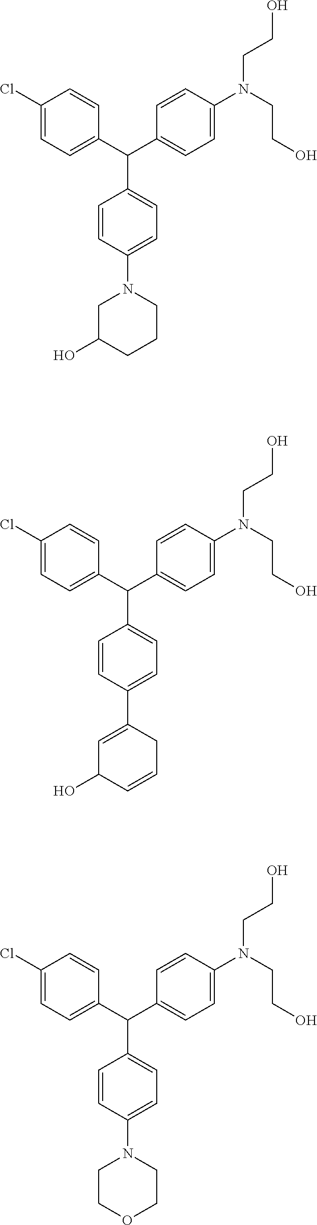 [SMILES: OCCN(CCO)c1ccc(C(c2ccc(Cl)cc2)c2ccc(N3CCCC(O)C3)cc2)cc1 ; OCCN(CCO)c1ccc(C(c2ccc(Cl)cc2)c2ccc(C3=CC(O)C=CC3)cc2)cc1 ; OCCN(CCO)c1ccc(C(c2ccc(Cl)cc2)c2ccc(N3CCOCC3)cc2)cc1]

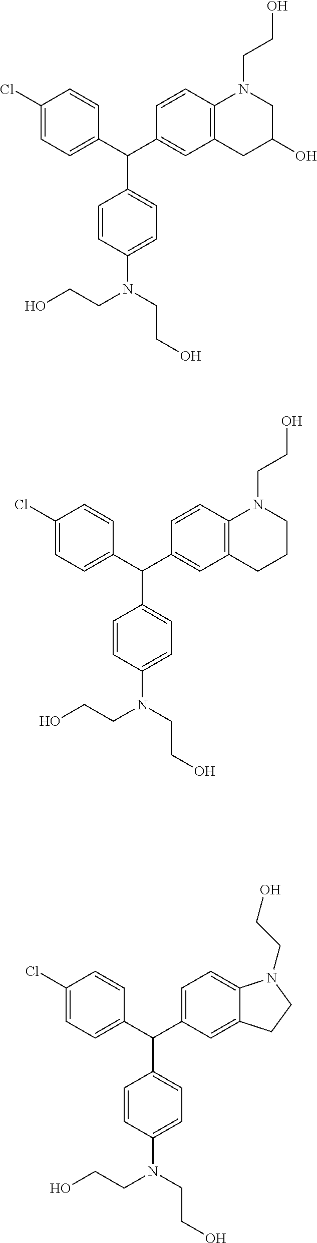 [SMILES: OCCN(CCO)c1ccc(C(c2ccc(Cl)cc2)c2ccc3c(c2)CC(O)CN3CCO)cc1 ; OCCN(CCO)c1ccc(C(c2ccc(Cl)cc2)c2ccc3c(c2)CCCN3CCO)cc1 ; OCCN(CCO)c1ccc(C(c2ccc(Cl)cc2)c2ccc3c(c2)CCN3CCO)cc1]

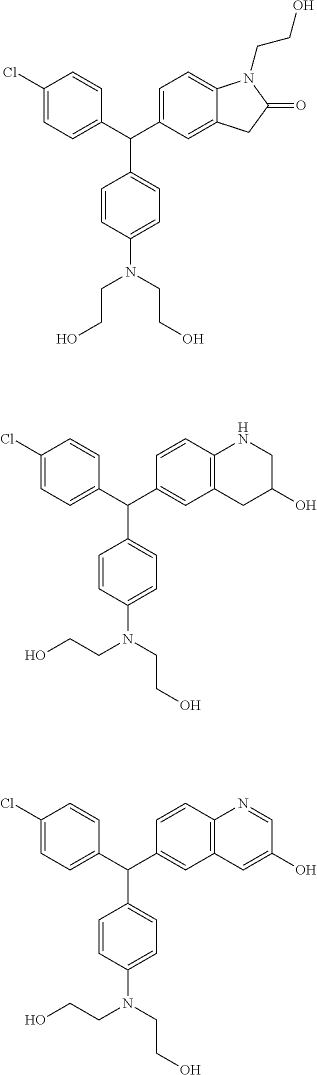 [SMILES: O=C1Cc2cc(C(c3ccc(Cl)cc3)c3ccc(N(CCO)CCO)cc3)ccc2N1CCO ; OCCN(CCO)c1ccc(C(c2ccc(Cl)cc2)c2ccc3c(c2)CC(O)CN3)cc1 ; OCCN(CCO)c1ccc(C(c2ccc(Cl)cc2)c2ccc3ncc(O)cc3c2)cc1]

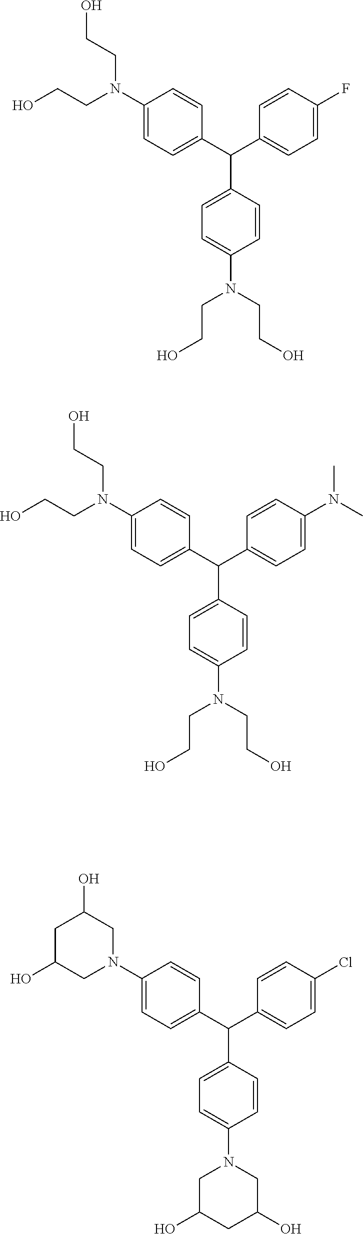 [SMILES: OCCN(CCO)c1ccc(C(c2ccc(F)cc2)c2ccc(N(CCO)CCO)cc2)cc1 ; CN(C)c1ccc(C(c2ccc(N(CCO)CCO)cc2)c2ccc(N(CCO)CCO)cc2)cc1 ; OC1CC(O)CN(c2ccc(C(c3ccc(Cl)cc3)c3ccc(N4CC(O)CC(O)C4)cc3)cc2)C1]

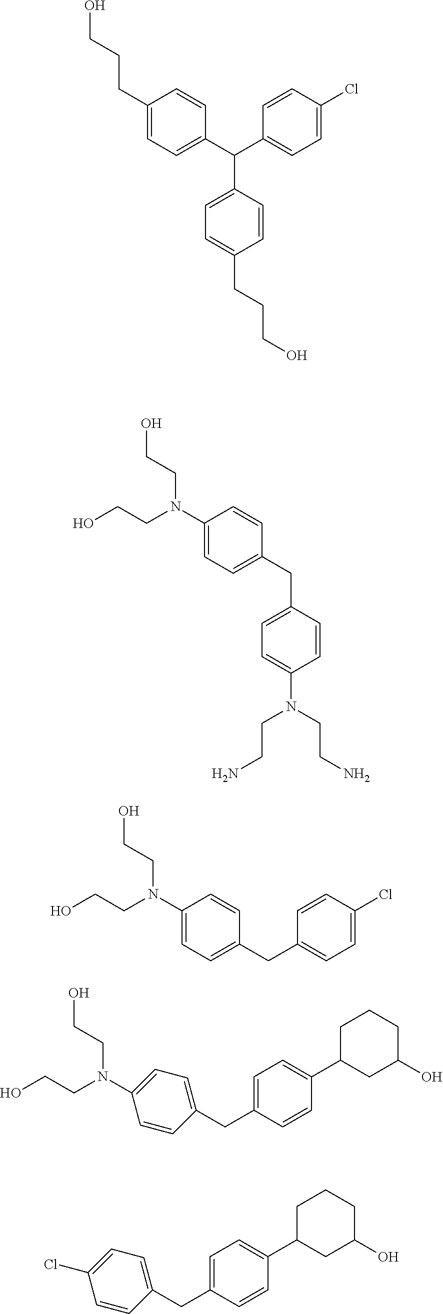 [SMILES: OCCCc1ccc(C(c2ccc(Cl)cc2)c2ccc(CCCO)cc2)cc1 ; NCCN(CCN)c1ccc(Cc2ccc(N(CCO)CCO)cc2)cc1 ; OCCN(CCO)c1ccc(Cc2ccc(Cl)cc2)cc1 ; OCCN(CCO)c1ccc(Cc2ccc(C3CCCC(O)C3)cc2)cc1 ; OC1CCCC(c2ccc(Cc3ccc(Cl)cc3)cc2)C1]

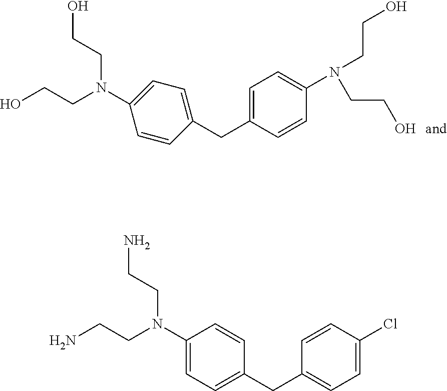 [SMILES: OCCN(CCO)c1ccc(Cc2ccc(N(CCO)CCO)cc2)cc1 ; NCCN(CCN)c1ccc(Cc2ccc(Cl)cc2)cc1]

or a pharmaceutically acceptable salt, ester, prodrug, or solvate thereof.

In one aspect, the present application discloses a compound of Formula (II):

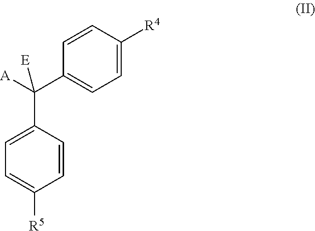

wherein: each of R⁴ and R⁵ is independently halo, —NR^{C}R^{D}, optionally substituted heterocycloalkyl, optionally substituted cycloalkyl, or optionally substituted aryl; A is —H, C₁-C₆ alkyl, C₂-C₆ alkenyl, C₂-C₆ alkynyl, optionally substituted aryl, or optionally substituted heteroaryl; E is —H or halo; and each of R^{C} and R^{D} is independently —H, C₁-C₆ alkyl, C₁-C₆ aminoalkylene, C₂-C₆ aminoalkenyl, C₂-C₆ aminoalkynyl, C₁-C₆ hydroxyalkylene, C₂-C₆ hydroxyalkenyl, or C₂-C₆ hydroxyalkynyl; or a pharmaceutically acceptable salt, ester, prodrug, or solvate thereof.

In one embodiment, the compound does not have the formula:

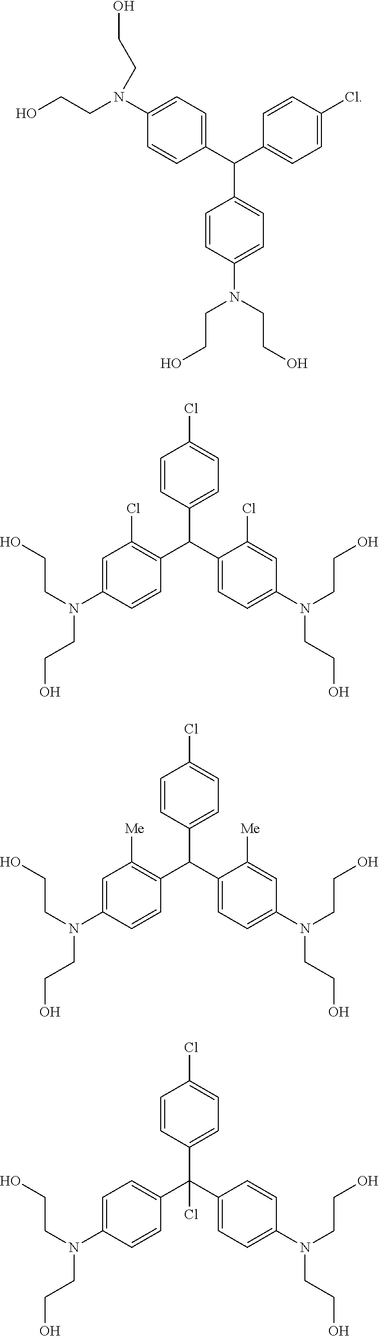

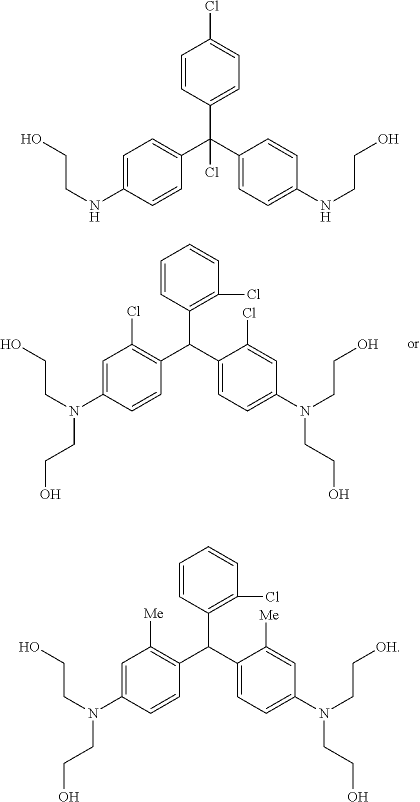 [SMILES: OCCNc1ccc(C(Cl)(c2ccc(Cl)cc2)c2ccc(NCCO)cc2)cc1 ; OCCN(CCO)c1ccc(C(c2ccccc2Cl)c2ccc(N(CCO)CCO)cc2Cl)c(Cl)c1 ; Cc1cc(N(CCO)CCO)ccc1C(c1ccc(N(CCO)CCO)cc1C)c1ccccc1Cl]

In one embodiment, each of R⁴ and R⁵ is independently —F, —Cl, —NR^{C}R^{D}, optionally substituted morpholinyl, optionally substituted thiomorpholinyl, optionally substituted piperazinyl, optionally substituted piperidinyl, optionally substituted pyrrolidinyl, optionally substituted cyclohexadienyl, or optionally substituted phenyl; A is —H, C₁-C₆ alkyl, optionally substituted phenyl, or optionally substituted bicyclic heteroaryl; E is —H or —Cl; and each of R^{C} and R^{D} is independently —H, C₁-C₆ alkyl, C₁-C₆ aminoalkyl, or C₁-C₆ hydroxyalkyl; or a pharmaceutically acceptable salt, ester, prodrug, or solvate thereof. In one variation, each of R⁴ and R⁵ is independently —F, —Cl, —NR^{C}R^{D}, optionally substituted N-bound morpholinyl, optionally substituted N-bound piperidinyl, or optionally substituted cyclohexa-1,4-dienyl; A is —H, C₃-C₆ alkylene, optionally substituted phenyl, optionally substituted quinolinyl, or optionally substituted tetrahydroquinolinyl; E is —H; and each of R^{C} and R^{D} is independently —H, methyl, ethyl, C₂-C₄ aminoalkylene, or C₂-C₄ hydroxyalkylene; or a pharmaceutically acceptable salt, ester, prodrug, or solvate thereof.

In one embodiment, each of A and E is —H or a pharmaceutically acceptable salt, ester, prodrug, or solvate thereof. In another embodiment, A is C₃-C₆ alkylene and E is —H or a pharmaceutically acceptable salt, ester, prodrug, or solvate thereof. In yet another embodiment, A is quinolinyl substituted with one or more of —OH and C₁-C₆ hydroxyalkylene, and E is —H or a pharmaceutically acceptable salt, ester, prodrug, or solvate thereof. In one variation thereof, A is quinolinyl substituted with C₂-C₄ hydroxyalkylene, and E is —H or a pharmaceutically acceptable salt, ester, prodrug, or solvate thereof. In another embodiment, A is tetrahydroquinolinyl substituted with one or more of —OH and C₁-C₆ hydroxyalkylene, and E is —H or a pharmaceutically acceptable salt, ester, prodrug, or solvate thereof. In one variation thereof, A is tetrahydroquinolinyl substituted with C₂-C₄ hydroxyalkylene, and E is —H or a pharmaceutically acceptable salt, ester, prodrug, or solvate thereof. In another embodiment, A is optionally substituted phenyl, and E is —H or —Cl, or a pharmaceutically acceptable salt, ester, prodrug, or solvate thereof. In one variation thereof, A is substituted phenyl, wherein the substituent is selected from the group consisting of —Cl, -Me and —NR^{C}R^{D}, wherein each of R^{C} and R^{D} is independently —H, C₁-C₆ aminoalkylene, or C₁-C₆ hydroxyalkylene; or a pharmaceutically acceptable salt, ester, prodrug, or solvate thereof. In another variation, the substituent is —NR^{C}R^{D}, wherein each of R^{C} and R^{D} is C₂-C₄ aminoalkylene, or C₂-C₄ hydroxyalkylene; or a pharmaceutically acceptable salt, ester, prodrug, or solvate thereof. In another variation, each of R^{C} and R^{D} is —CH₂CH₂—OH or a pharmaceutically acceptable salt, ester, prodrug, or solvate thereof.

In one embodiment, the compound has a structural formula selected from the group consisting of:

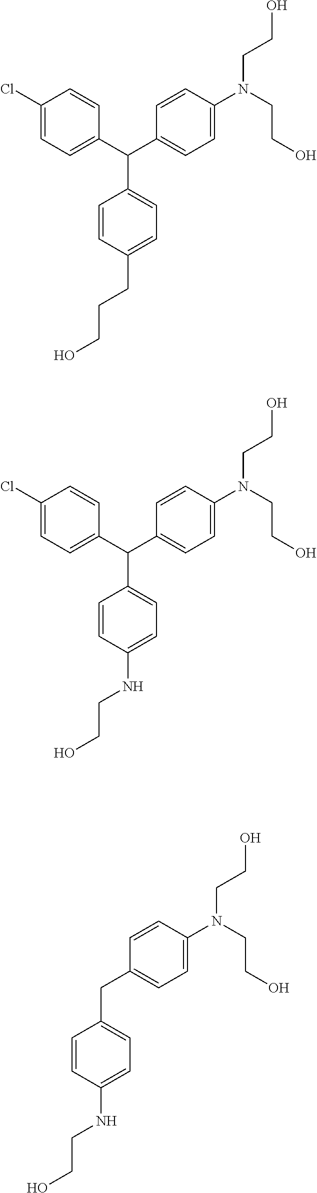 [SMILES: OCCCc1ccc(C(c2ccc(Cl)cc2)c2ccc(N(CCO)CCO)cc2)cc1 ; OCCNc1ccc(C(c2ccc(Cl)cc2)c2ccc(N(CCO)CCO)cc2)cc1 ; OCCNc1ccc(Cc2ccc(N(CCO)CCO)cc2)cc1]

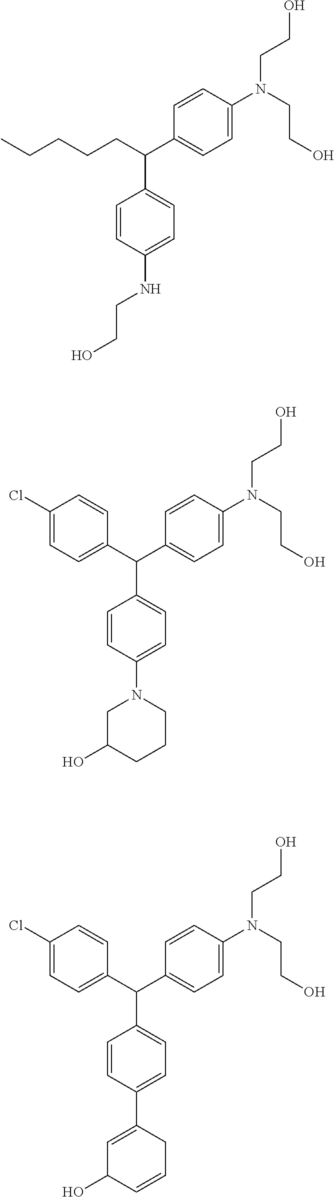 [SMILES: CCCCCC(c1ccc(NCCO)cc1)c1ccc(N(CCO)CCO)cc1 ; OCCN(CCO)c1ccc(C(c2ccc(Cl)cc2)c2ccc(N3CCCC(O)C3)cc2)cc1 ; OCCN(CCO)c1ccc(C(c2ccc(Cl)cc2)c2ccc(C3=CC(O)C=CC3)cc2)cc1]

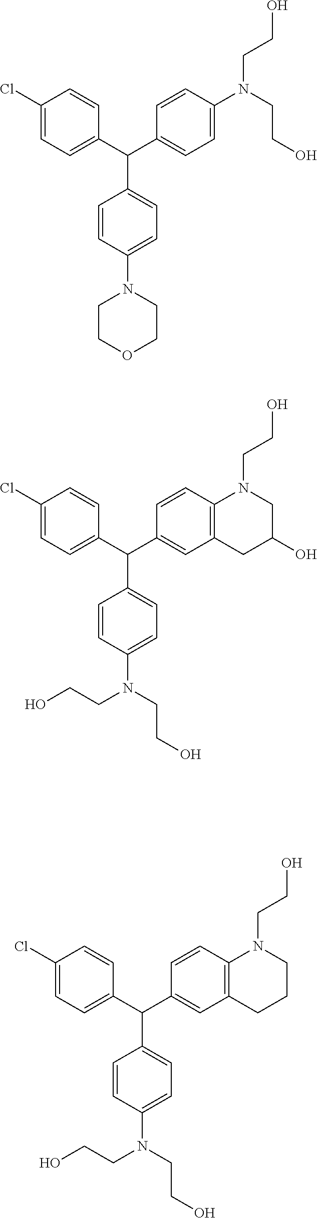 [SMILES: OCCN(CCO)c1ccc(C(c2ccc(Cl)cc2)c2ccc(N3CCOCC3)cc2)cc1 ; OCCN(CCO)c1ccc(C(c2ccc(Cl)cc2)c2ccc3c(c2)CC(O)CN3CCO)cc1 ; OCCN(CCO)c1ccc(C(c2ccc(Cl)cc2)c2ccc3c(c2)CCCN3CCO)cc1]

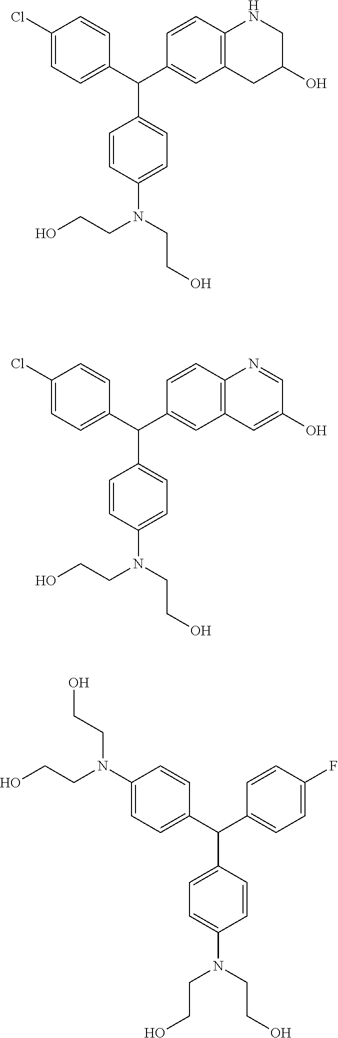 [SMILES: OCCN(CCO)c1ccc(C(c2ccc(Cl)cc2)c2ccc3c(c2)CC(O)CN3)cc1 ; OCCN(CCO)c1ccc(C(c2ccc(Cl)cc2)c2ccc3ncc(O)cc3c2)cc1 ; OCCN(CCO)c1ccc(C(c2ccc(F)cc2)c2ccc(N(CCO)CCO)cc2)cc1]

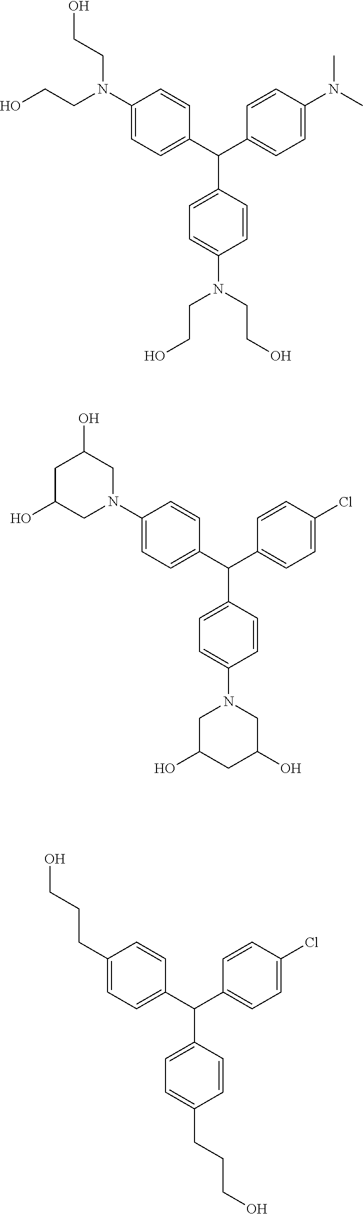 [SMILES: CN(C)c1ccc(C(c2ccc(N(CCO)CCO)cc2)c2ccc(N(CCO)CCO)cc2)cc1 ; OC1CC(O)CN(c2ccc(C(c3ccc(Cl)cc3)c3ccc(N4CC(O)CC(O)C4)cc3)cc2)C1 ; OCCCc1ccc(C(c2ccc(Cl)cc2)c2ccc(CCCO)cc2)cc1]

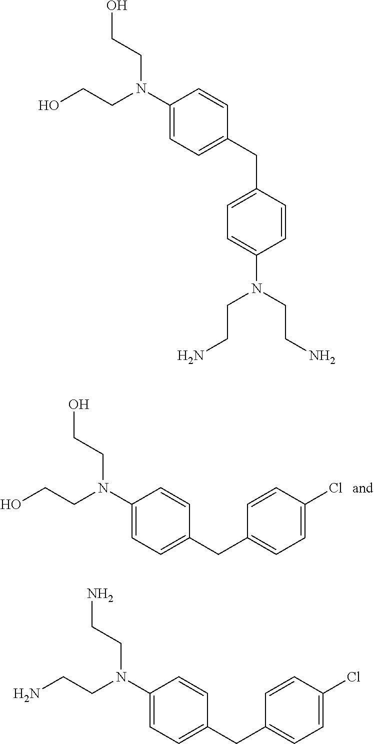 [SMILES: NCCN(CCN)c1ccc(Cc2ccc(N(CCO)CCO)cc2)cc1 ; OCCN(CCO)c1ccc(Cc2ccc(Cl)cc2)cc1 ; NCCN(CCN)c1ccc(Cc2ccc(Cl)cc2)cc1]

or a pharmaceutically acceptable salt, ester, prodrug, or solvate thereof.

In one aspect, the present application discloses a method of treating a disorder that can be treated by contacting, activating or inhibiting a TrkB receptor in a subject comprising administering to the subject in need thereof an effective amount of a compound having binding and/or modulation specificity for a TrkB receptor molecule, for example when the compound is a compound disclosed herein. In one embodiment, the disorder is selected from the group consisting of Alzheimer's disease, Huntington's disease, amyotrophic lateral sclerosis, Rett syndrome, epilepsy, Parkinson's disease, spinal cord injury, stroke, hypoxia, ischemia, brain injury, diabetic neuropathy, peripheral neuropathy, nerve transplantation complications, motor neuron disease, multiple sclerosis, HIV dementia, peripheral nerve injury, hearing loss, depression, obesity, metabolic syndrome, pain, cancer, and other conditions involving degeneration or dysfunction of cells expressing TrkB.

In one aspect, the present application discloses a method of treating a disorder that can be treated by contacting, activating or inhibiting a TrkB receptor in a subject, comprising administering to the subject in need thereof an effective amount of a compound having binding and/or modulation specificity for a TrkB receptor molecule. In one embodiment, the compound has a binding and/or modulation specificity for a TrkB receptor molecule and a TrkA or TrkC receptor molecule.

In another aspect, the present application disclosed a method of treating a disorder that can be treated by contacting, activating or inhibiting a TrkB receptor in a subject, comprising administering to the subject in need thereof an effective amount of a compound of the invention. In one embodiment, a compound of the invention is selected from Group I:

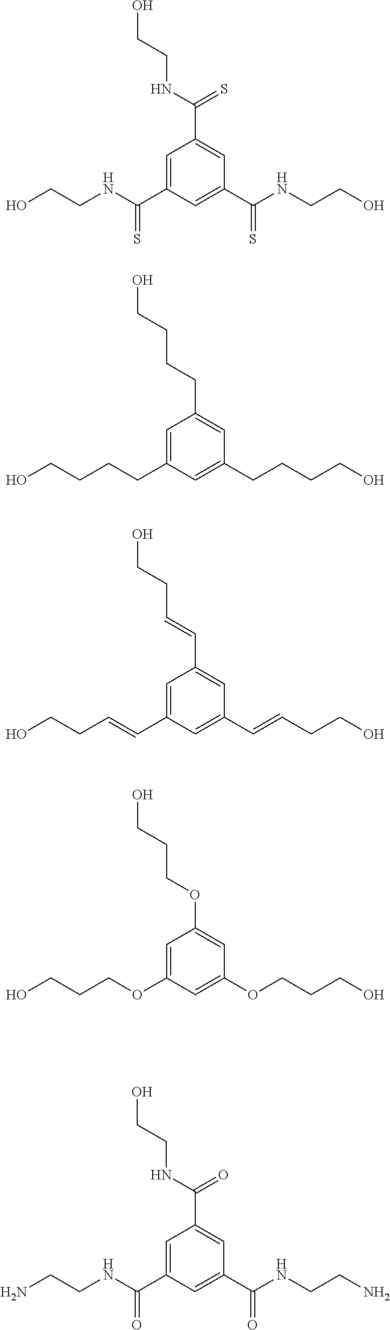

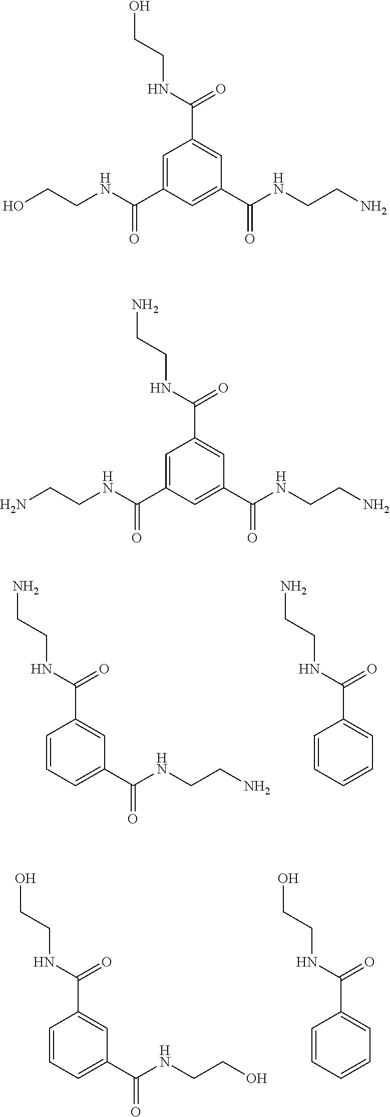 [SMILES: NCCNC(=O)c1cc(C(=O)NCCO)cc(C(=O)NCCO)c1 ; NCCNC(=O)c1cc(C(=O)NCCN)cc(C(=O)NCCN)c1 ; NCCNC(=O)c1cccc(C(=O)NCCN)c1 ; NCCNC(=O)c1ccccc1 ; O=C(NCCO)c1cccc(C(=O)NCCO)c1 ; O=C(NCCO)c1ccccc1]

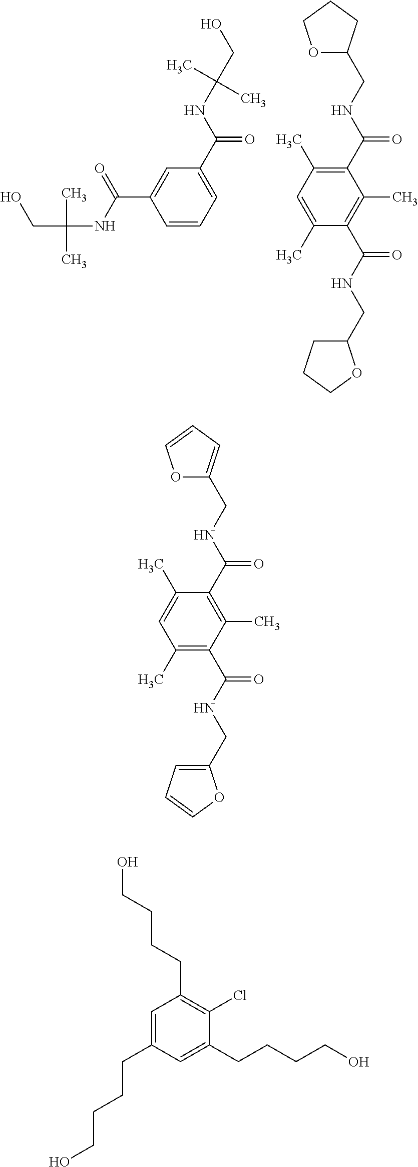 [SMILES: CC(C)(CO)NC(=O)c1cccc(C(=O)NC(C)(C)CO)c1 ; Cc1cc(C)c(C(=O)NCC2CCCO2)c(C)c1C(=O)NCC1CCCO1 ; Cc1cc(C)c(C(=O)NCc2ccco2)c(C)c1C(=O)NCc1ccco1 ; OCCCCc1cc(CCCCO)c(Cl)c(CCCCO)c1]

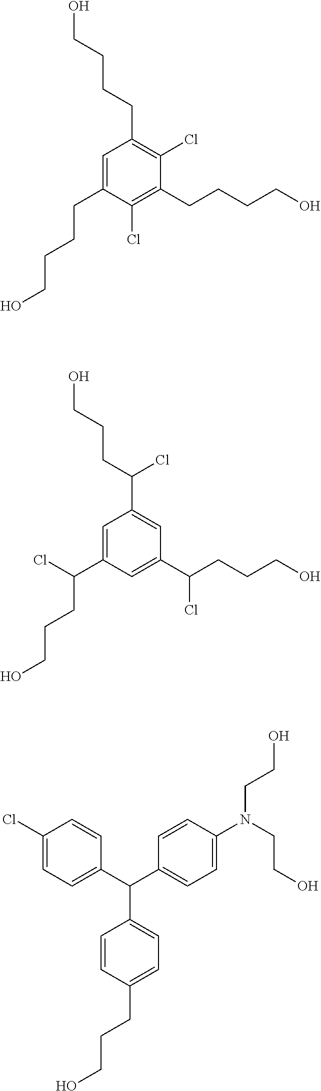 [SMILES: OCCCCc1cc(CCCCO)c(Cl)c(CCCCO)c1Cl ; OCCCC(Cl)c1cc(C(Cl)CCCO)cc(C(Cl)CCCO)c1 ; OCCCc1ccc(C(c2ccc(Cl)cc2)c2ccc(N(CCO)CCO)cc2)cc1]

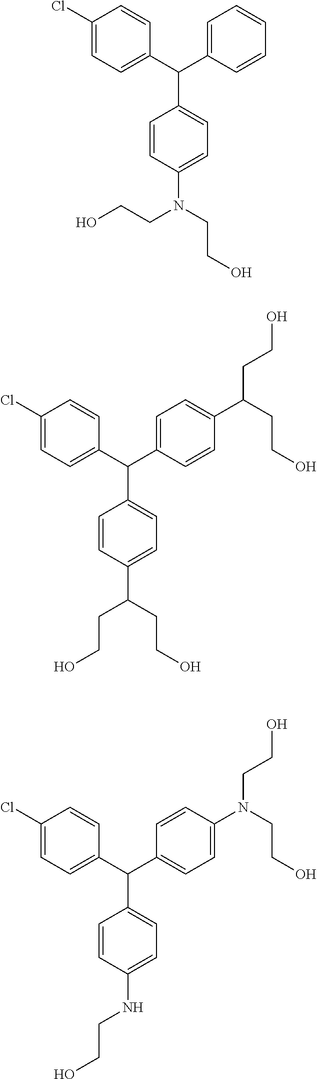 [SMILES: OCCN(CCO)c1ccc(C(c2ccccc2)c2ccc(Cl)cc2)cc1 ; OCCC(CCO)c1ccc(C(c2ccc(Cl)cc2)c2ccc(C(CCO)CCO)cc2)cc1 ; OCCNc1ccc(C(c2ccc(Cl)cc2)c2ccc(N(CCO)CCO)cc2)cc1]

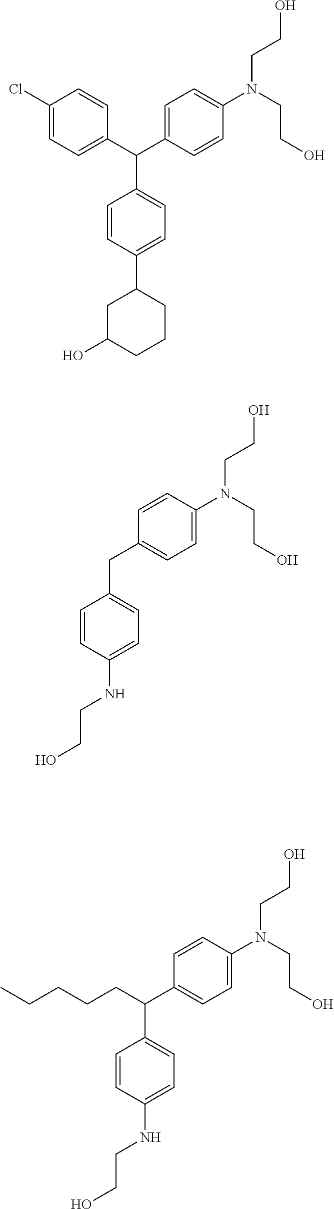 [SMILES: OCCN(CCO)c1ccc(C(c2ccc(Cl)cc2)c2ccc(C3CCCC(O)C3)cc2)cc1 ; OCCNc1ccc(Cc2ccc(N(CCO)CCO)cc2)cc1 ; CCCCCC(c1ccc(NCCO)cc1)c1ccc(N(CCO)CCO)cc1]

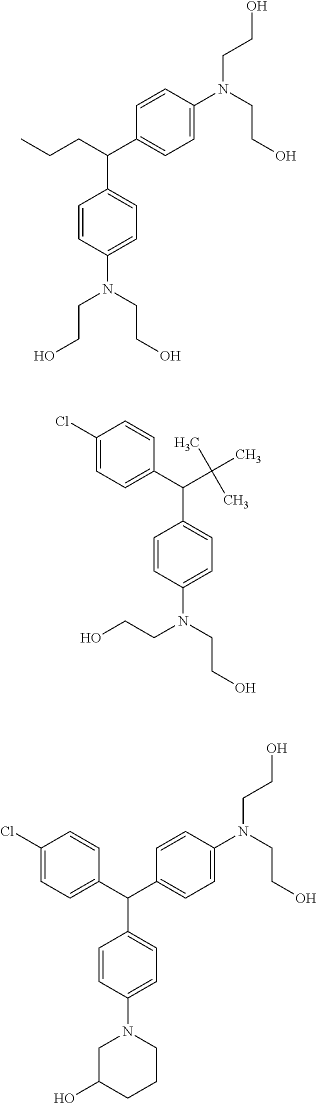 [SMILES: CCCC(c1ccc(N(CCO)CCO)cc1)c1ccc(N(CCO)CCO)cc1 ; CC(C)(C)C(c1ccc(Cl)cc1)c1ccc(N(CCO)CCO)cc1 ; OCCN(CCO)c1ccc(C(c2ccc(Cl)cc2)c2ccc(N3CCCC(O)C3)cc2)cc1]

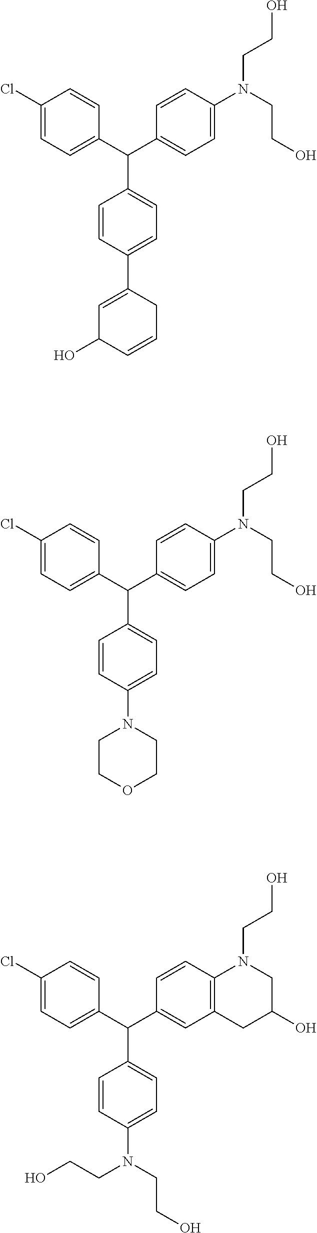 [SMILES: OCCN(CCO)c1ccc(C(c2ccc(Cl)cc2)c2ccc(C3=CC(O)C=CC3)cc2)cc1 ; OCCN(CCO)c1ccc(C(c2ccc(Cl)cc2)c2ccc(N3CCOCC3)cc2)cc1 ; OCCN(CCO)c1ccc(C(c2ccc(Cl)cc2)c2ccc3c(c2)CC(O)CN3CCO)cc1]

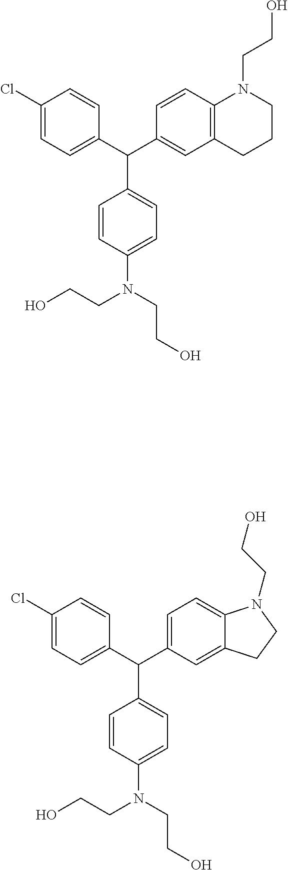 [SMILES: OCCN(CCO)c1ccc(C(c2ccc(Cl)cc2)c2ccc3c(c2)CCCN3CCO)cc1 ; OCCN(CCO)c1ccc(C(c2ccc(Cl)cc2)c2ccc3c(c2)CCN3CCO)cc1]

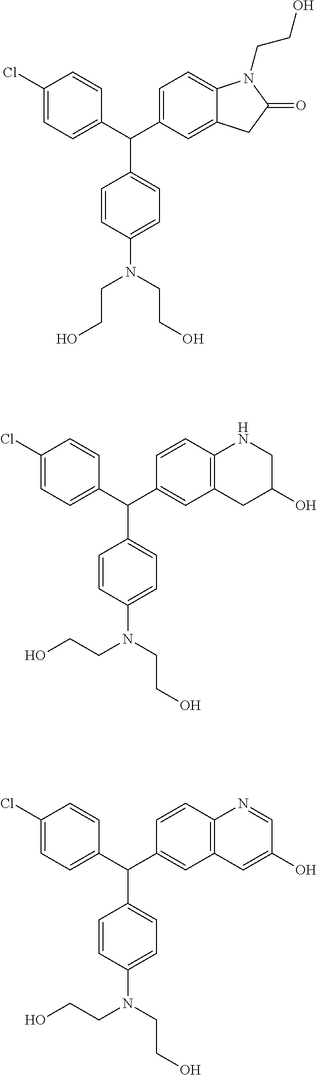 [SMILES: O=C1Cc2cc(C(c3ccc(Cl)cc3)c3ccc(N(CCO)CCO)cc3)ccc2N1CCO ; OCCN(CCO)c1ccc(C(c2ccc(Cl)cc2)c2ccc3c(c2)CC(O)CN3)cc1 ; OCCN(CCO)c1ccc(C(c2ccc(Cl)cc2)c2ccc3ncc(O)cc3c2)cc1]

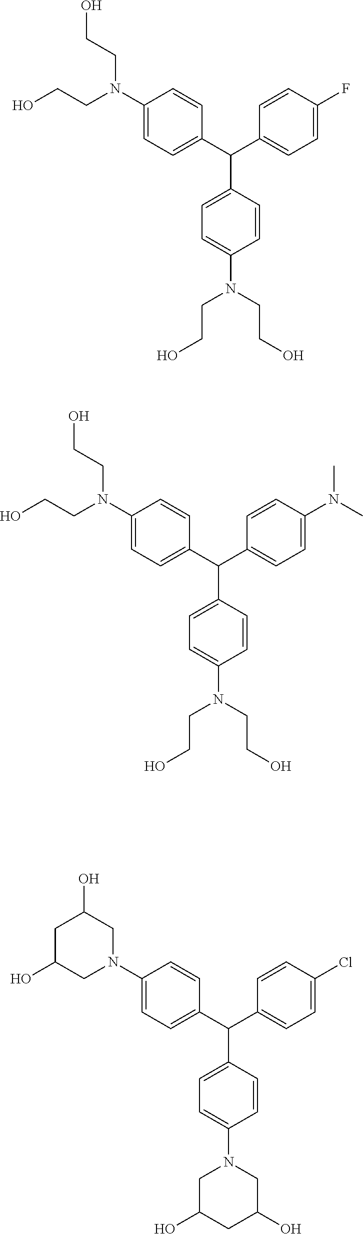 [SMILES: OCCN(CCO)c1ccc(C(c2ccc(F)cc2)c2ccc(N(CCO)CCO)cc2)cc1 ; CN(C)c1ccc(C(c2ccc(N(CCO)CCO)cc2)c2ccc(N(CCO)CCO)cc2)cc1 ; OC1CC(O)CN(c2ccc(C(c3ccc(Cl)cc3)c3ccc(N4CC(O)CC(O)C4)cc3)cc2)C1]

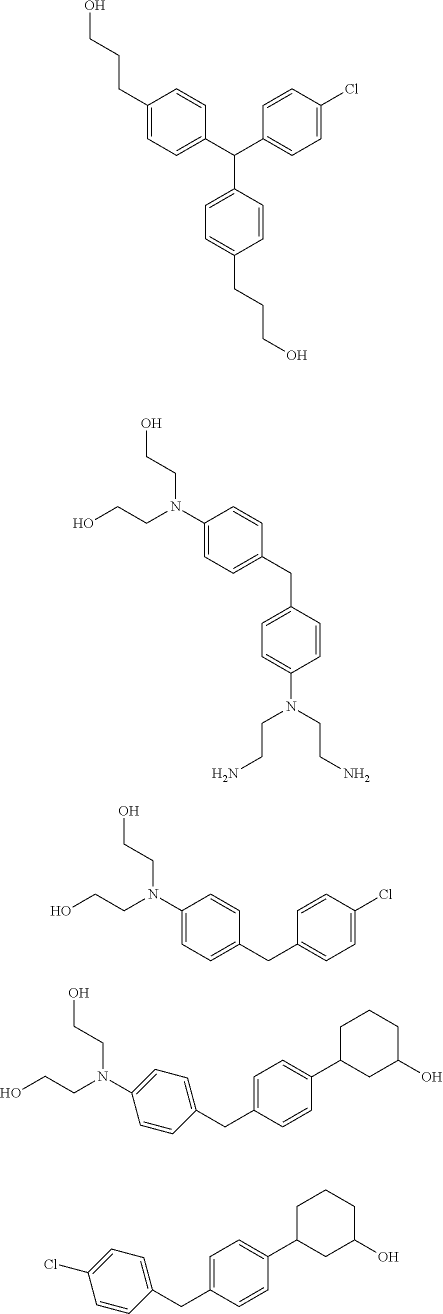 [SMILES: OCCCc1ccc(C(c2ccc(Cl)cc2)c2ccc(CCCO)cc2)cc1 ; NCCN(CCN)c1ccc(Cc2ccc(N(CCO)CCO)cc2)cc1 ; OCCN(CCO)c1ccc(Cc2ccc(Cl)cc2)cc1 ; OCCN(CCO)c1ccc(Cc2ccc(C3CCCC(O)C3)cc2)cc1 ; OC1CCCC(c2ccc(Cc3ccc(Cl)cc3)cc2)C1]

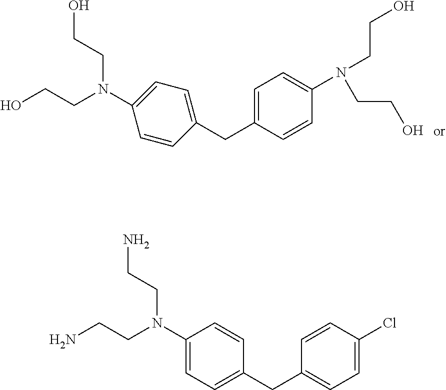 [SMILES: OCCN(CCO)c1ccc(Cc2ccc(N(CCO)CCO)cc2)cc1 ; NCCN(CCN)c1ccc(Cc2ccc(Cl)cc2)cc1]

or a pharmaceutically acceptable salt, ester, prodrug, or solvate thereof.

In another aspect, the present application discloses a method of treating a disorder that can be treated by contacting, activating or inhibiting a TrkB receptor in a subject in need of treatment thereof, comprising administering to the subject an effective amount of a compound selected from Group II:

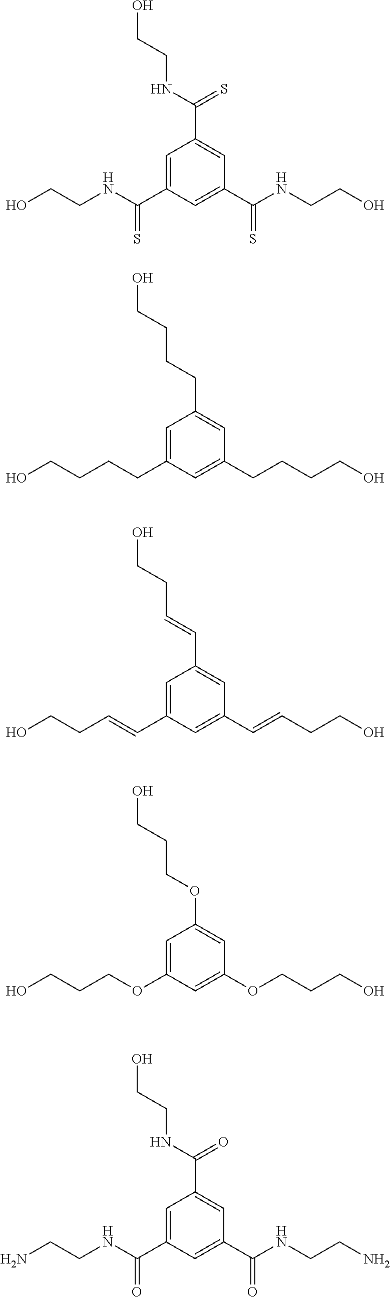

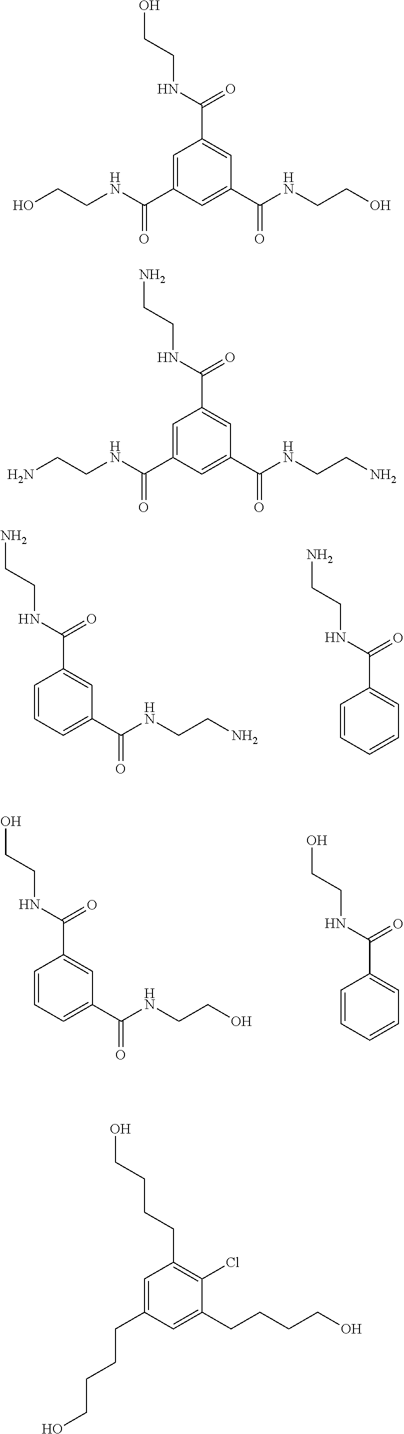

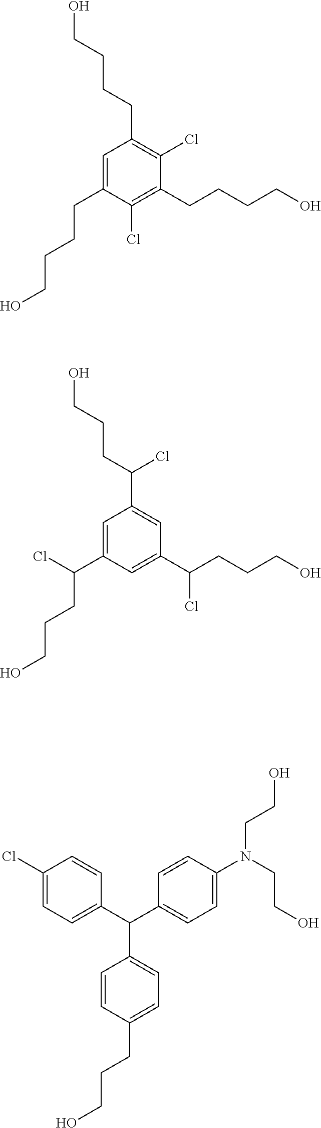 [SMILES: OCCCCc1cc(CCCCO)c(Cl)c(CCCCO)c1Cl ; OCCCC(Cl)c1cc(C(Cl)CCCO)cc(C(Cl)CCCO)c1 ; OCCCc1ccc(C(c2ccc(Cl)cc2)c2ccc(N(CCO)CCO)cc2)cc1]

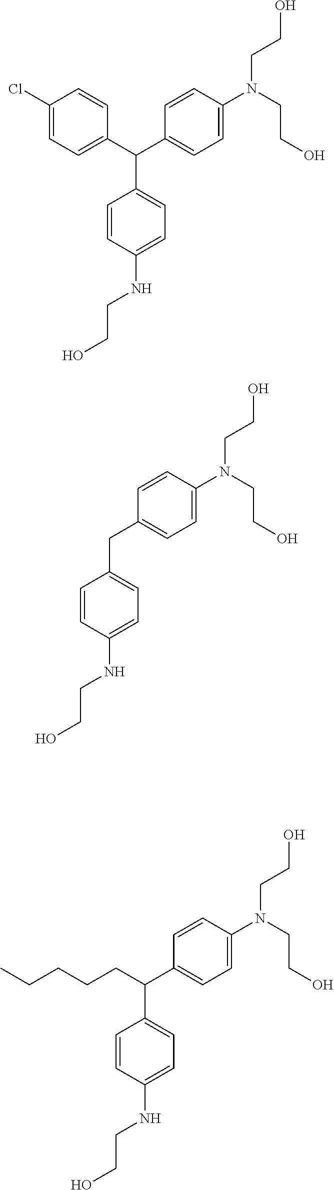 [SMILES: OCCNc1ccc(C(c2ccc(Cl)cc2)c2ccc(N(CCO)CCO)cc2)cc1 ; OCCNc1ccc(Cc2ccc(N(CCO)CCO)cc2)cc1 ; CCCCCC(c1ccc(NCCO)cc1)c1ccc(N(CCO)CCO)cc1]

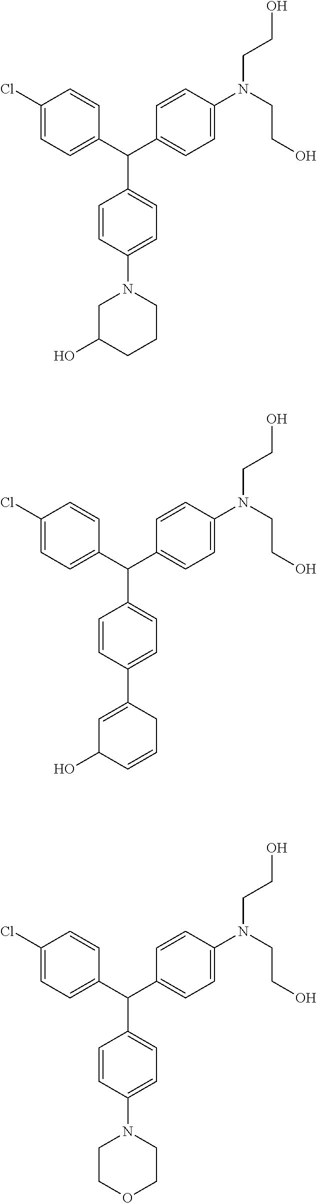 [SMILES: OCCN(CCO)c1ccc(C(c2ccc(Cl)cc2)c2ccc(N3CCCC(O)C3)cc2)cc1 ; OCCN(CCO)c1ccc(C(c2ccc(Cl)cc2)c2ccc(C3=CC(O)C=CC3)cc2)cc1 ; OCCN(CCO)c1ccc(C(c2ccc(Cl)cc2)c2ccc(N3CCOCC3)cc2)cc1]

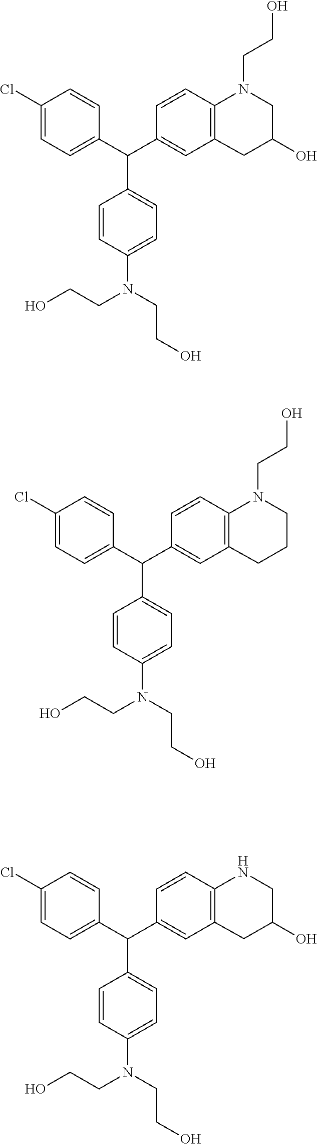 [SMILES: OCCN(CCO)c1ccc(C(c2ccc(Cl)cc2)c2ccc3c(c2)CC(O)CN3CCO)cc1 ; OCCN(CCO)c1ccc(C(c2ccc(Cl)cc2)c2ccc3c(c2)CCCN3CCO)cc1 ; OCCN(CCO)c1ccc(C(c2ccc(Cl)cc2)c2ccc3c(c2)CC(O)CN3)cc1]

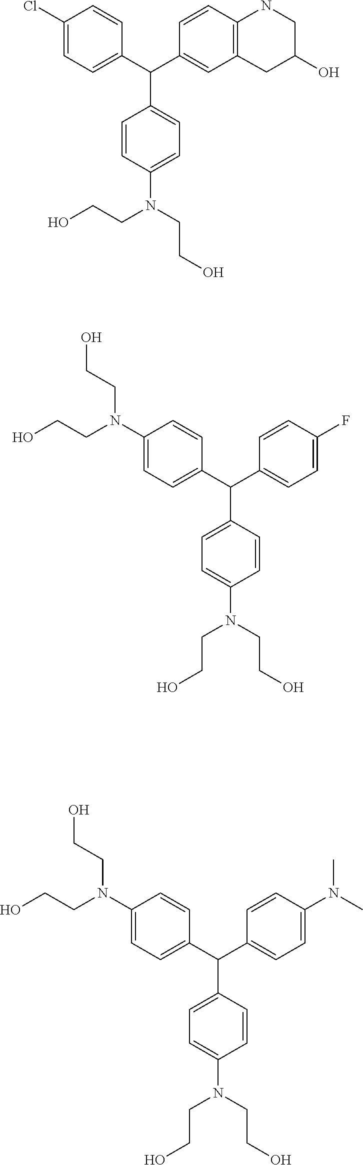 [SMILES: OCCN(CCO)c1ccc(C(c2ccc(Cl)cc2)c2ccc3c(c2)CC(O)CN3)cc1 ; OCCN(CCO)c1ccc(C(c2ccc(F)cc2)c2ccc(N(CCO)CCO)cc2)cc1 ; CN(C)c1ccc(C(c2ccc(N(CCO)CCO)cc2)c2ccc(N(CCO)CCO)cc2)cc1]

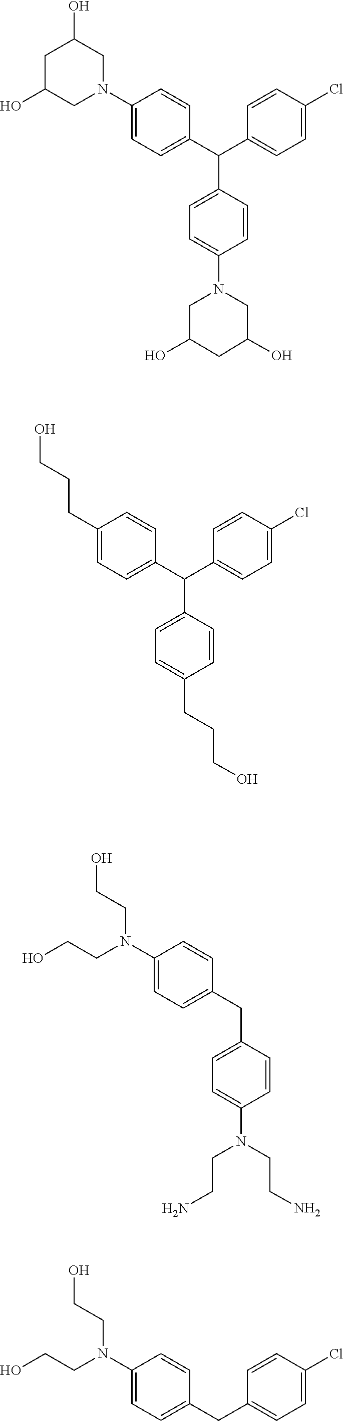 [SMILES: OC1CC(O)CN(c2ccc(C(c3ccc(Cl)cc3)c3ccc(N4CC(O)CC(O)C4)cc3)cc2)C1 ; OCCCc1ccc(C(c2ccc(Cl)cc2)c2ccc(CCCO)cc2)cc1 ; NCCN(CCN)c1ccc(Cc2ccc(N(CCO)CCO)cc2)cc1 ; OCCN(CCO)c1ccc(Cc2ccc(Cl)cc2)cc1]

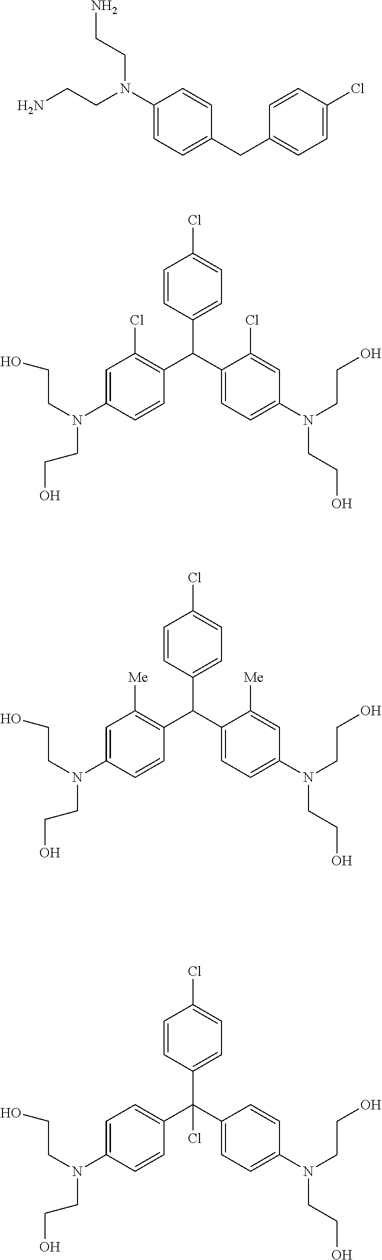 [SMILES: NCCN(CCN)c1ccc(Cc2ccc(Cl)cc2)cc1 ; OCCN(CCO)c1ccc(C(c2ccc(Cl)cc2)c2ccc(N(CCO)CCO)cc2Cl)c(Cl)c1 ; Cc1cc(N(CCO)CCO)ccc1C(c1ccc(Cl)cc1)c1ccc(N(CCO)CCO)cc1C ; OCCN(CCO)c1ccc(C(Cl)(c2ccc(Cl)cc2)c2ccc(N(CCO)CCO)cc2)cc1]

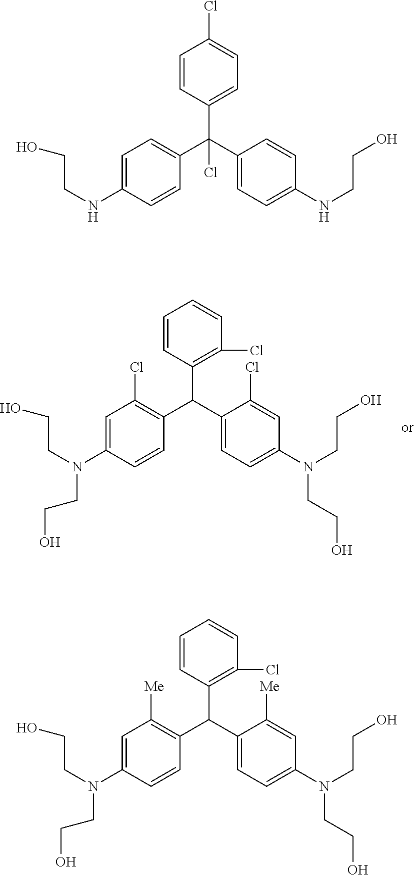 [SMILES: OCCNc1ccc(C(Cl)(c2ccc(Cl)cc2)c2ccc(NCCO)cc2)cc1 ; OCCN(CCO)c1ccc(C(c2ccccc2Cl)c2ccc(N(CCO)CCO)cc2Cl)c(Cl)c1 ; Cc1cc(N(CCO)CCO)ccc1C(c1ccc(N(CCO)CCO)cc1C)c1ccccc1Cl]

or a pharmaceutically acceptable salt, ester, prodrug, or solvate thereof.

In one variation of any disclosed aspect or embodiment, the disorder is selected from the group consisting of Alzheimer's disease, Huntington's disease, amyotrophic lateral sclerosis, Rett syndrome, epilepsy, Parkinson's disease, spinal cord injury, stroke, hypoxia, ischemia, brain injury, diabetic neuropathy, peripheral neuropathy, nerve transplantation complications, motor neuron disease, multiple sclerosis, HIV dementia, peripheral nerve injury, hearing loss, depression, obesity, metabolic syndrome, pain, cancer, and other conditions involving degeneration or dysfunction of cells expressing TrkB.

In another aspect, the present application discloses a method of treating a disorder that can be treated by contacting, activating or inhibiting a TrkB receptor in a subject, comprising administering to the subject in need thereof an effective amount of a compound having a formula selected from Group I or II.

In one variation of any disclosed aspect or embodiment, the compounds of the invention do not include any of:

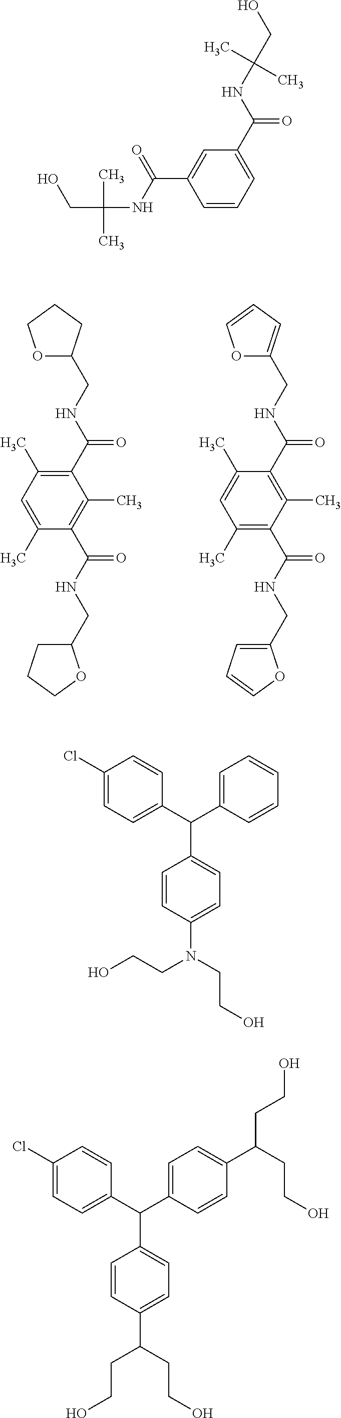 [SMILES: CC(C)(CO)NC(=O)c1cccc(C(=O)NC(C)(C)CO)c1 ; Cc1cc(C)c(C(=O)NCC2CCCO2)c(C)c1C(=O)NCC1CCCO1 ; Cc1cc(C)c(C(=O)NCc2ccco2)c(C)c1C(=O)NCc1ccco1 ; OCCN(CCO)c1ccc(C(c2ccccc2)c2ccc(Cl)cc2)cc1 ; OCCC(CCO)c1ccc(C(c2ccc(Cl)cc2)c2ccc(C(CCO)CCO)cc2)cc1]

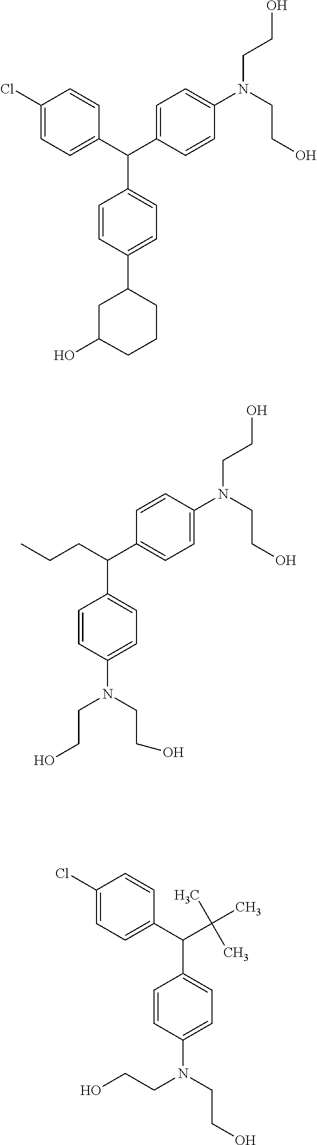 [SMILES: OCCN(CCO)c1ccc(C(c2ccc(Cl)cc2)c2ccc(C3CCCC(O)C3)cc2)cc1 ; CCCC(c1ccc(N(CCO)CCO)cc1)c1ccc(N(CCO)CCO)cc1 ; CC(C)(C)C(c1ccc(Cl)cc1)c1ccc(N(CCO)CCO)cc1]

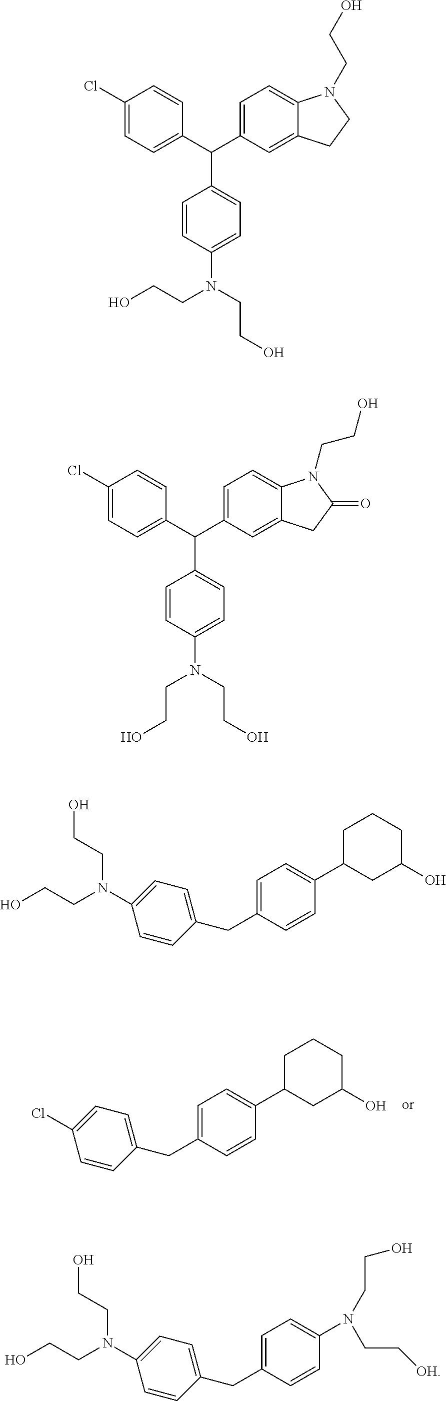 [SMILES: OCCN(CCO)c1ccc(C(c2ccc(Cl)cc2)c2ccc3c(c2)CCN3CCO)cc1 ; O=C1Cc2cc(C(c3ccc(Cl)cc3)c3ccc(N(CCO)CCO)cc3)ccc2N1CCO ; OCCN(CCO)c1ccc(Cc2ccc(C3CCCC(O)C3)cc2)cc1 ; OC1CCCC(c2ccc(Cc3ccc(Cl)cc3)cc2)C1 ; OCCN(CCO)c1ccc(Cc2ccc(N(CCO)CCO)cc2)cc1]

In one aspect, the present application discloses a method of facilitating cell survival comprising treating a TrkB-expressing cell with a compound having binding and/or modulation specificity for a TrkB receptor molecule. In one embodiment, the compound has a binding and/or modulation specificity for a TrkB receptor molecule and a TrkA or TrkC receptor molecule.

In another aspect, the present application discloses a method of facilitating cell survival comprising treating a TrkB-expressing cell with a compound of the invention. In one embodiment, the compound has a formula selected from Group I as defined above. In another embodiment, the compound has a formula selected from Group II as defined above; in yet another embodiment the compound has a formula selected from Group I or Group II. In one variation of any aspect or embodiment, the TrkB-expressing cell is a neuronal cell.

In another aspect, the present application discloses a method for activating a TrkB receptor molecule comprising contacting a cell containing a TrkB receptor molecule with an effective amount of a compound having binding and/or modulation specificity for a TrkB receptor molecule. In one embodiment, the compound has a binding and/or modulation specificity for a TrkB receptor molecule and a TrkA or TrkC receptor molecule.

In one aspect, the present application discloses a method for activating a TrkB receptor molecule comprising contacting a cell containing a TrkB receptor molecule with an effective amount of a compound of the invention. In one embodiment, the compound has a formula selected from Group I as defined above. In another embodiment, the compound has a formula selected from Group II as defined above; in yet another embodiment the compound has a formula selected from Group I or Group II.

In one aspect, the present application discloses a pharmaceutical formulation comprising a unit dose of an active ingredient and a pharmaceutical grade carrier, wherein the active ingredient is selected from the group consisting of a compound of the invention.

In another aspect, the present application discloses a pharmaceutical formulation comprising a unit dose of an active ingredient and a pharmaceutical grade carrier, wherein the active ingredient is a compound having a formula selected from Group I as defined above. In another aspect, the compound has a formula selected from Group II as defined above; in yet another aspect the compound has a formula selected from Group I or Group II.

In one embodiment, the formulation is a formulation for parenteral or oral administration. In another embodiment of any aspect or variation disclosed herein the formulation further comprises a second active ingredient.

### Formulations

For the purposes of this invention, the compounds may be administered by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The compounds disclosed herein can be formulated in accordance with the routine procedures adapted for desired administration route. Accordingly, the compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compounds can further be formulated for topical administration. Suitable topical formulations include one or more compounds in the form of a liquid, lotion, cream or gel. Topical administration can be accomplished by application directly on the treatment area. For example, such application can be accomplished by rubbing the formulation (such as a lotion or gel) onto the skin of the treatment area, or by spray application of a liquid formulation onto the treatment area.

In some formulations, bioimplant materials can be coated with the compounds so as to improve interaction between cells and the implant.

Formulations of the compounds can contain minor amounts of wetting or emulsifying agents, or pH buffering agents. The formulations comprising the compound can be a liquid solution, suspension, emulsion, tablet, pill, capsule, sustained release formulation, or powder.

The compounds can be formulated as a suppository, with traditional binders and carriers such as triglycerides.

Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Oral formulations can include standard carriers such as pharmaceutical grades of mannitol, lactose, starch, magnesium stearate, polyvinyl pyrrolidone, sodium saccharine, cellulose, magnesium carbonate, etc. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax maybe employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

The pharmaceutical formulations comprising the compounds of the present application can include an agent which controls release of the compound, thereby providing a timed or sustained release compound.

### Carriers

Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions.

Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

### Salts

It is also to be understood that the disclosed compounds can further comprise pharmaceutically acceptable salts.

Such salts include, but are not limited to, pharmaceutically acceptable acid addition salts, pharmaceutically acceptable base addition salts, pharmaceutically acceptable metal salts, ammonium and alkylated ammonium salts.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like.

Examples of metal salts include lithium, sodium, potassium, magnesium salts and the like.

Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like.

Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa.

### Methods of Use

The presently disclosed subject matter provides novel methods of treating disorders, including, but not limited to, neurological disorders (e.g., neurodegenerative disorders) and in a subject. More particularly, the methods of the presently disclosed subject matter involve the administration of a compound having binding and/or modulation specificity for a TrkB receptor molecule in a subject to treat a disorder. The compound can be administered in an amount effective to induce survival signaling and/or to upregulate neural function. The compound can also be used to stimulate desired mechanisms of non-neural cells. The compound can also be used to partially or fully block endogenous BDNF.

The disorder to be treated can be any condition that is mediated, at least in part, by binding of neurotrophins to the TrkB receptor, and conditions wherein the TrkB receptor is present, though not necessarily causally linked to the condition. Neurotrophins can be present or absent in the condition. Such disorders include, but are not limited to, Alzheimer's disease, Huntington's disease, amyotrophic lateral sclerosis (ALS), Rett syndrome, epilepsy, Parkinson's disease, spinal cord injury, stroke, hypoxia, ischemia, brain injury, diabetic neuropathy, peripheral neuropathy, nerve transplantation complications, multiple sclerosis, peripheral nerve injury, conditions involving degeneration or dysfunction of cells expressing TrkB. The disorder to be treated further includes depression, obesity, and ischemic conditions of peripheral tissues. TrkB involvement has been linked to a number of disorders, including, but not limited to Alzheimer's disease, Huntington's disease, Parkinson's disease, Rett syndrome, Motor neuron disease, depression, ischemic stroke, HIV dementia, multiple sclerosis, spinal cord injury, hearing loss, obesity, diabetes, metabolic syndrome, peripheral tissue ischemia, epilepsy, pain and cancer.

The presently disclosed subject matter further provides for methods of facilitating cell survival or function, including both neural cells and non-neural cells. Representative neural cells include, but are not limited to, hippocampal pyramidal cells, cortical cells, striatal cells, substantial nigra cells, motor neuron cells, Purkinje cells, dorsal root ganglia cells. Non-neuronal cells include, but are not limited to, vascular endothelial cells. The methods can comprise treating a neural or non-neural cell with a compound having binding or modulation specificity for a TrkB receptor molecule, whereby the compound induces survival signaling and/or upregulation of cell function.

The BDNF mimetics of the present invention can be used in both in vivo and in vitro settings. In some embodiments, the BDNF mimetics can be used as a cost saving alternative to BDNF in in vitro methods. In some embodiments, the BDNF mimetics can be used in methods related to stem cells. Thus, in some embodiments, the BDNF mimetics can be used for maintaining stem cells in an undifferentiated state or to induce stem cell differentiation. By way of example, a BDNF mimetic as disclosed herein can be used in methods currently available in the art that employ BDNF (Huang, E. J., Reichardt, L. F. (2003) Annu Rev Biochem 72, 609-642; Banker, G., Goslin, K. (Eds.) (1998) Culturing Nerve Cells, Chapters 10 and 14 (Cambridge, Mass.: The MIT Press)), except with the substitution of the BDNF mimetic.

### Administration

The presently disclosed subject matter provides methods of administering compounds having binding and/or modulation specificity for a TrkB receptor compound in order to ameliorate a disorder mediated by TrkB binding or modulation in a subject. The method can comprise administering to a subject an effective amount of a compound having binding and/or modulation specificity for a TrkB receptor, such as any of the compounds disclosed herein.

The presently disclosed subject matter provides methods of administering compounds having binding and/or modulation specificity for a TrkB receptor compound in order to ameliorate a disorder mediated by TrkB binding or modulation in a subject. The method can comprise administering to a subject an effective amount of a compound having binding and/or modulation specificity for a TrkB receptor, such as any of the compounds disclosed herein.

As used herein, administering can be effected or performed using any of the various methods known to those skilled in the art. The compound can be administered, for example, subcutaneously, intravenously, parenterally, intraperitoneally, intradermally, intramuscularly, topically, enteral (e.g., orally), rectally, nasally, buccally, sublingually, vaginally, by inhalation spray, by drug pump or via an implanted reservoir in dosage formulations containing conventional non-toxic, physiologically acceptable carriers or vehicles.

Further, the presently disclosed compounds can be administered to a localized area in need of treatment. This can be achieved by, for example, and not by way of limitation, local infusion during surgery, topical application, transdermal patches, by injection, by catheter, by suppository, or by implant (the implant can optionally be of a porous, non-porous, or gelatinous material), including membranes, such as sialastic membranes or fibers.

The form in which the compound is administered (e.g., syrup, elixir, capsule, tablet, solution, foams, emulsion, gel, sol) will depend in part on the route by which it is administered. For example, for mucosal (e.g., oral mucosa, rectal, intestinal mucosa, bronchial mucosa) administration, nose drops, aerosols, inhalants, nebulizers, eye drops or suppositories can be used. The compounds and agents disclosed herein can be administered together with other biologically active agents, such as analgesics, anti-inflammatory agents, anesthetics and other agents which can control one or more symptoms or causes of a TrkB mediated disorder.

Additionally, administration can comprise administering to the subject a plurality of dosages over a suitable period of time. Such administration regimens can be determined according to routine methods, upon a review of the instant disclosure.

In some embodiments, administration comprises administering to the subject a dose or plurality of dosages to achieve a compound concentration in a cell or in a cell microenvironment of between about 0.10 μM and about 50 μM.

The compounds of the presently disclosed subject matter can be employed as the sole active agent in a pharmaceutical or can be used in combination (e.g., administered proximate in time to each other or even in the same formulation) with other active ingredients, e.g., neurotrophins, or other factors or drugs which can facilitate neural survival or axonal growth in neurodegenerative diseases. For example, synergistic effects can be provided by administering a compound having binding and/or modulation specificity for a TrkB receptor molecule to a subject with a second compound having binding and/or modulation specificity for a p75^{NTR} molecule.

### Dosage

Compounds of the invention are generally administered orally in a total daily dose of about 0.01 mg/kg/dose to about 100 mg/kg/dose. Alternately the dose can be from about 0.1 mg/kg/dose to about 10 mg/kg/dose; or about 1 mg/kg/dose to 10 mg/kg/dose. In some dosages, the compounds disclosed herein are administered at about 5 mg/kg/dose. Time release preparations may be employed or the dose may be administered in as many divided doses as is convenient. When other methods are used (e.g. intravenous administration), compounds are administered to the affected tissue at a rate from about 0.05 to about 10 mg/kg/hour, alternately from about 0.1 to about 1 mg/kg/hour. Such rates are easily maintained when these compounds are intravenously administered as discussed herein. Generally, topically administered formulations are administered in a dose of about 0.5 mg/kg/dose to about 10 mg/kg/dose range. Alternately, topical formulations are administered at a dose of about 1 mg/kg/dose to about 7.5 mg/kg/dose or even about 1 mg/kg/dose to about 5 mg/kg/dose.

Drug doses can also be given in milligrams per square meter of body surface area rather than body weight, as this method achieves a good correlation to certain metabolic and excretionary functions. Moreover, body surface area can be used as a common denominator for drug dosage in adults and children as well as in different animal species (Freireich et al., (1966) Cancer Chemother Rep. 50, 219-244). Briefly, to express a mg/kg dose in any given species as the equivalent mg/sq m dose, the dosage is multiplied by the appropriate km factor. In an adult human, 100 mg/kg is equivalent to 100 mg/kg×37 kg/sq m=3700 mg/m².

It will be appreciated by one of skill in the art that dosage range will depend on the particular compound, and its potency. The dosage range is understood to be large enough to produce the desired effect in which the neurological disorder and the symptoms associated therewith are ameliorated and/or survival of the neural cells is achieved, but not be so large as to cause unmanageable adverse side effects. It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art. The dosage can also be adjusted by the individual physician in the event of any complication. No unacceptable toxicological effects are expected when compounds disclosed herein are used in accordance with the present application.

An effective amount of the compounds disclosed herein comprise amounts sufficient to produce a measurable biological response. Actual dosage levels of active ingredients in a therapeutic compound of the presently disclosed subject matter can be varied so as to administer an amount of the active compound that is effective to achieve the desired therapeutic response for a particular subject and/or application. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

Further with respect to the methods of the presently disclosed subject matter, a preferred subject is a vertebrate subject. A preferred vertebrate is warm-blooded; a preferred warm-blooded vertebrate is a mammal. The subject treated by the presently disclosed methods is desirably a human, although it is to be understood that the principles of the presently disclosed subject matter indicate effectiveness with respect to all vertebrate species which are to included in the term “subject.” In this context, a vertebrate is understood to be any vertebrate species in which treatment of a neurodegenerative disorder is desirable. As used herein, the term “subject” includes both human and animal subjects. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos or as pets (including parrots), as well as fowl, and more particularly domesticated fowl, i.e., poultry, such as turkeys, chickens, ducks, geese, guinea fowl, and the like, as they are also of economical importance to humans. Thus, also provided is the treatment of livestock, including, but not limited to, domesticated swine, ruminants, ungulates, horses (including race horses), poultry, and the like.

### EXAMPLES

### Example

### General Syntheses

Standard procedures and chemical transformation and related methods are well known to one skilled in the art, and such methods and procedures have been described, for example, in standard references such as Fiesers' Reagents for Organic Synthesis, John Wiley and Sons, New York, N.Y., 2002; Organic Reactions, vols. 1-83, John Wiley and Sons, New York, N.Y., 2006; March J. and Smith M., Advanced Organic Chemistry, 6th ed., John Wiley and Sons, New York, N.Y.; and Larock R. C., Comprehensive Organic Transformations, Wiley-VCH Publishers, New York, 1999. All texts and references cited herein are incorporated by reference in their entirety.

Reactions using compounds having functional groups may be performed on compounds with functional groups that may be protected. A “protected” compound or derivatives means derivatives of a compound where one or more reactive site or sites or functional groups are blocked with protecting groups. Protected derivatives are useful in the preparation of the compounds of the present invention or in themselves; the protected derivatives may be the biologically active agent. An example of a comprehensive text listing suitable protecting groups may be found in T. W. Greene, Protecting Groups in Organic Synthesis, 3rd edition, John Wiley & Sons, Inc. 1999.

### Example

### Preparation of Compounds of the Invention

A number of the compounds of the invention are commercially available, including BDNF2-4-3, BDNF2-4-4, BDNF4-4-1, BDNF4-4-2, BDNF4-4-3, BDNF5-1-1, BDNF5-1-2, BDNF5-1-3, BDNF5-1-4, BDNF5-1-5, BDNF5-1-5, and BDNF5-1-6; BDNF2-4-5 can be obtained via substitution chemistry from BDNF2-4-4.

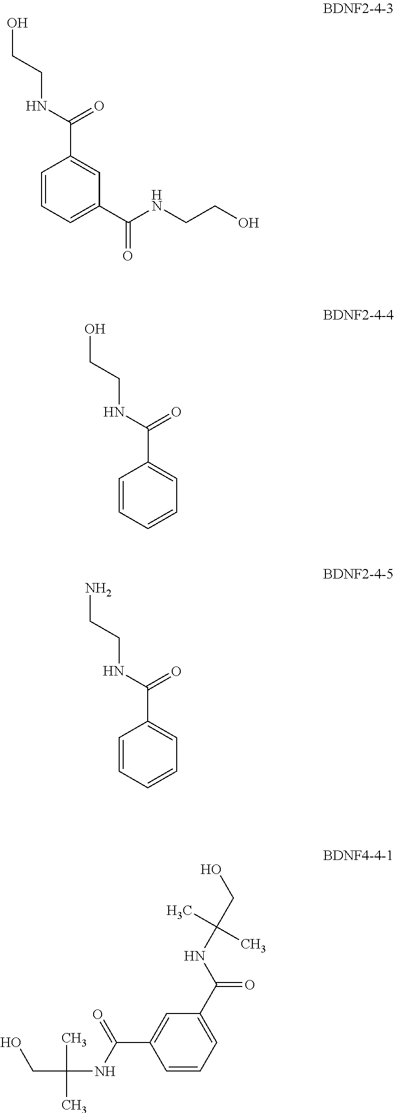 [SMILES: O=C(NCCO)c1cccc(C(=O)NCCO)c1 ; O=C(NCCO)c1ccccc1 ; NCCNC(=O)c1ccccc1 ; CC(C)(CO)NC(=O)c1cccc(C(=O)NC(C)(C)CO)c1]

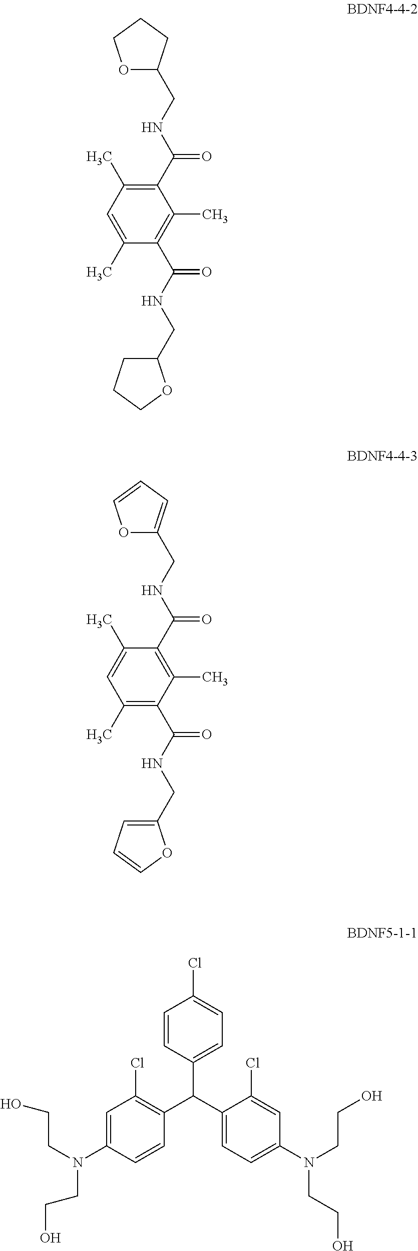 [SMILES: Cc1cc(C)c(C(=O)NCC2CCCO2)c(C)c1C(=O)NCC1CCCO1 ; Cc1cc(C)c(C(=O)NCc2ccco2)c(C)c1C(=O)NCc1ccco1 ; OCCN(CCO)c1ccc(C(c2ccc(Cl)cc2)c2ccc(N(CCO)CCO)cc2Cl)c(Cl)c1]

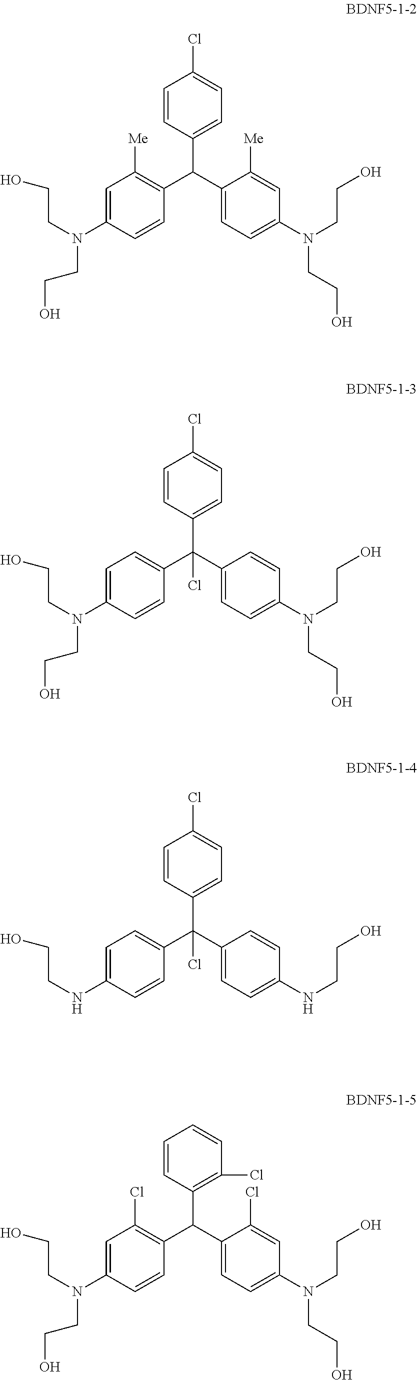 [SMILES: Cc1cc(N(CCO)CCO)ccc1C(c1ccc(Cl)cc1)c1ccc(N(CCO)CCO)cc1C ; OCCN(CCO)c1ccc(C(Cl)(c2ccc(Cl)cc2)c2ccc(N(CCO)CCO)cc2)cc1 ; OCCNc1ccc(C(Cl)(c2ccc(Cl)cc2)c2ccc(NCCO)cc2)cc1 ; OCCN(CCO)c1ccc(C(c2ccccc2Cl)c2ccc(N(CCO)CCO)cc2Cl)c(Cl)c1]

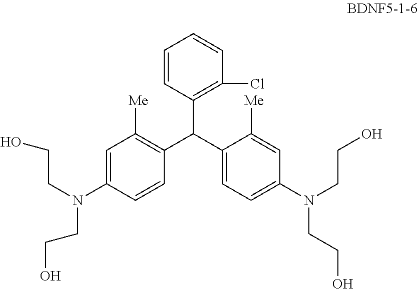 [SMILES: Cc1cc(N(CCO)CCO)ccc1C(c1ccc(N(CCO)CCO)cc1C)c1ccccc1Cl]

A variety of compounds can be prepared according to Schemes 1-8 disclosed below. Other compounds, including BDNF3-4-3, BDNF3-10-6, BDNF3-10-9, BDNF4-10-3a, BDNF4-10-4-b, BDNF4-10-5a, BDNF4-10-6, BDNF3-10-11, BDNF3-10-13, BDNF3-10-15, and BDNF4-10-2a can be prepared according to the routes analogous to the synthetic schemes provided herein.

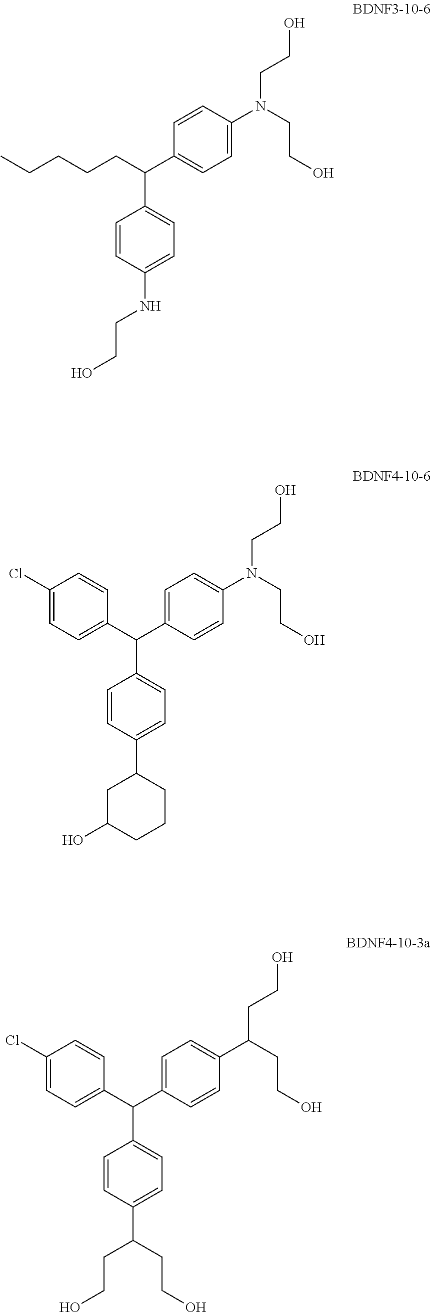 [SMILES: CCCCCC(c1ccc(NCCO)cc1)c1ccc(N(CCO)CCO)cc1 ; OCCN(CCO)c1ccc(C(c2ccc(Cl)cc2)c2ccc(C3CCCC(O)C3)cc2)cc1 ; OCCC(CCO)c1ccc(C(c2ccc(Cl)cc2)c2ccc(C(CCO)CCO)cc2)cc1]

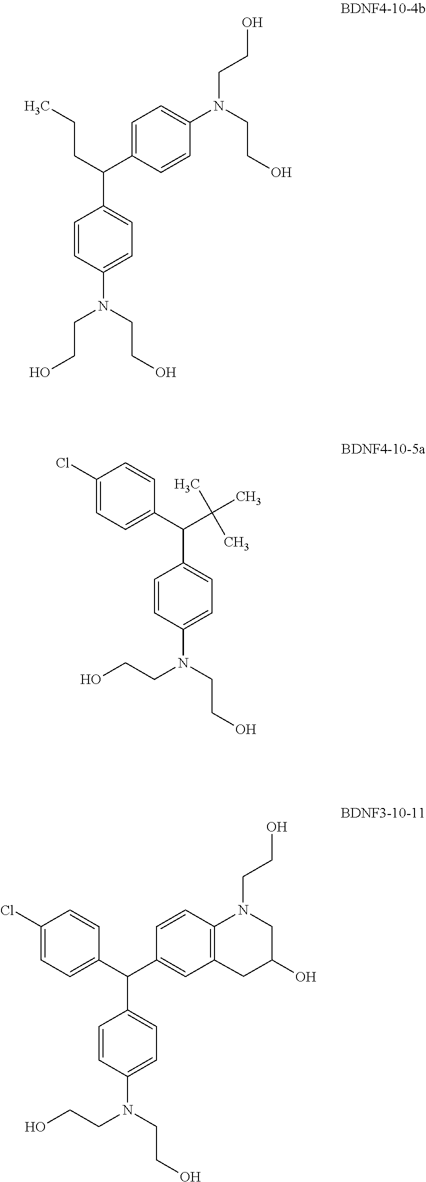 [SMILES: CCCC(c1ccc(N(CCO)CCO)cc1)c1ccc(N(CCO)CCO)cc1 ; CC(C)(C)C(c1ccc(Cl)cc1)c1ccc(N(CCO)CCO)cc1 ; OCCN(CCO)c1ccc(C(c2ccc(Cl)cc2)c2ccc3c(c2)CC(O)CN3CCO)cc1]

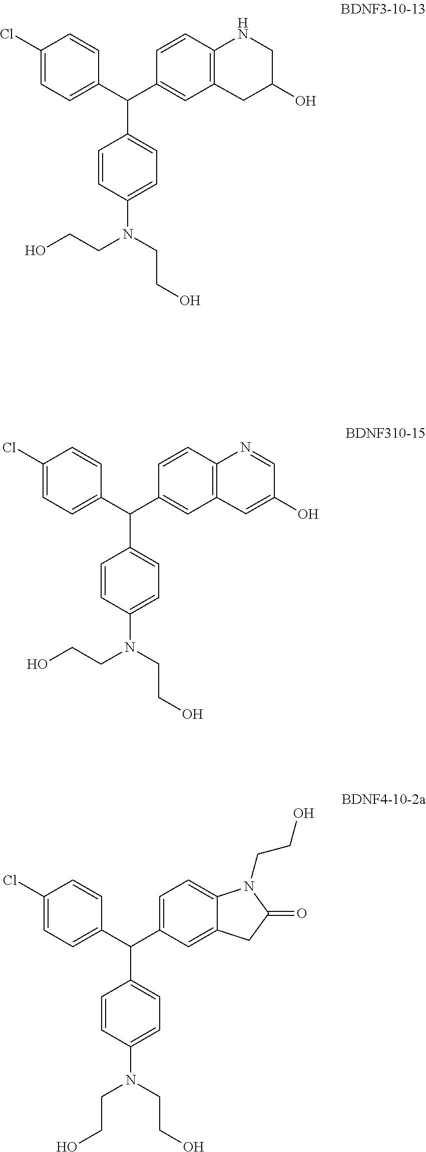 [SMILES: OCCN(CCO)c1ccc(C(c2ccc(Cl)cc2)c2ccc3c(c2)CC(O)CN3)cc1 ; OCCN(CCO)c1ccc(C(c2ccc(Cl)cc2)c2ccc3ncc(O)cc3c2)cc1 ; O=C1Cc2cc(C(c3ccc(Cl)cc3)c3ccc(N(CCO)CCO)cc3)ccc2N1CCO]

Some of compounds of the invention may be difficult to isolate, due to stability limitations, including BDNF3-4-3 and BDNF3-10-9.

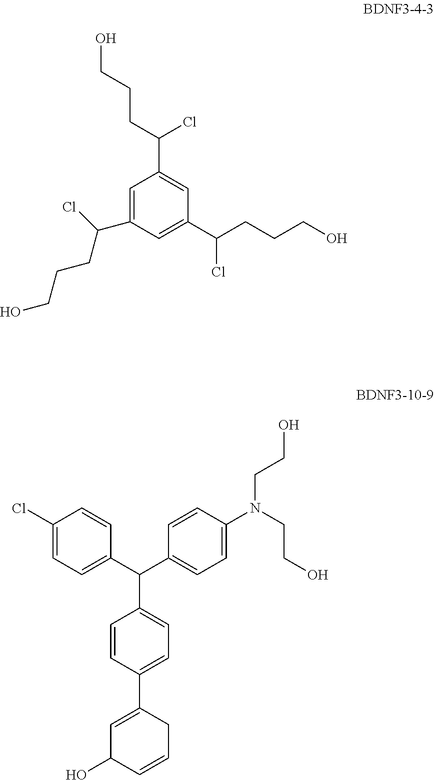 [SMILES: OCCCC(Cl)c1cc(C(Cl)CCCO)cc(C(Cl)CCCO)c1 ; OCCN(CCO)c1ccc(C(c2ccc(Cl)cc2)c2ccc(C3=CC(O)C=CC3)cc2)cc1]

Compound BDNF1-4-15-2 can be prepared as described in Scheme 1.

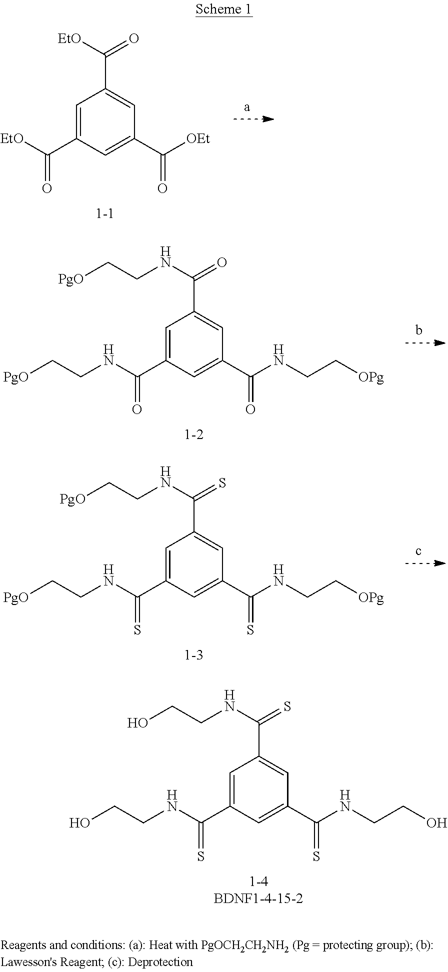

Compounds BDNF2-4-1, BDNF2-4-2, BDNF2-4-6, and BDNF2-4-7, can be prepared as generally described in Scheme 2, with the proper starting materials. In particular, the starting material of BDNF2-4-7 can be diethyl isophthalate, and in an initial step, chemistry at the 5-position is blocked by a protecting group, which is removed in the final step in the process.

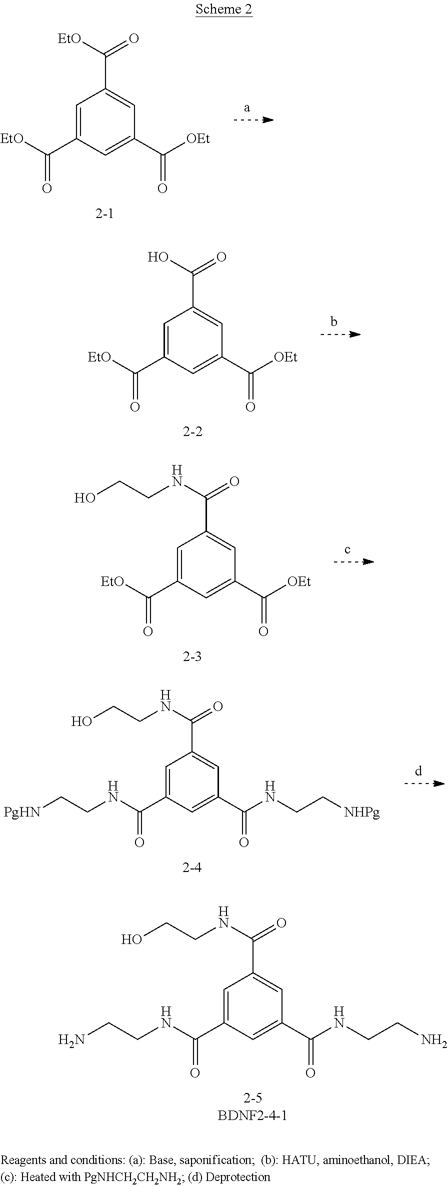

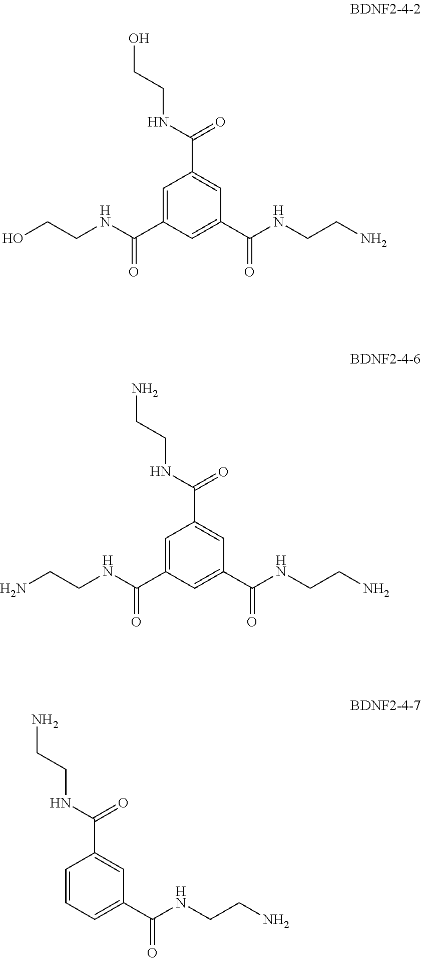 [SMILES: NCCNC(=O)c1cc(C(=O)NCCO)cc(C(=O)NCCO)c1 ; NCCNC(=O)c1cc(C(=O)NCCN)cc(C(=O)NCCN)c1 ; NCCNC(=O)c1cccc(C(=O)NCCN)c1]

Compounds BDNF3-4-1, BDNF3-4-2, and BDNF1-4-15-3 can be prepared as generally described in Scheme 3, with the proper starting materials.

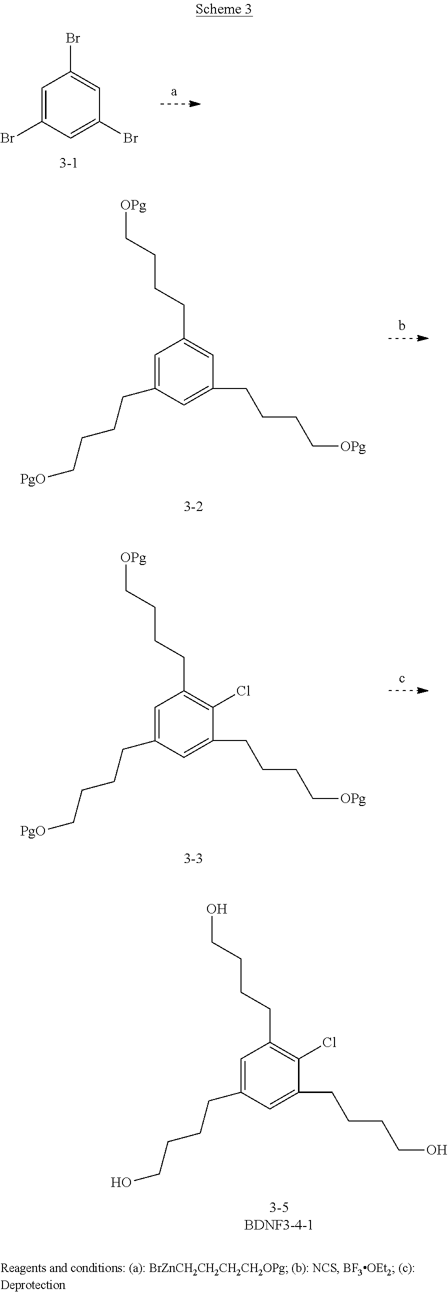

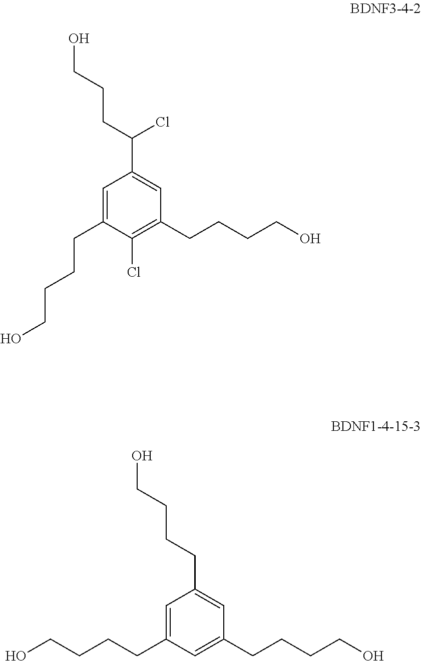 [SMILES: OCCCCc1cc(C(Cl)CCCO)cc(CCCCO)c1Cl ; OCCCCc1cc(CCCCO)cc(CCCCO)c1]

Compounds BDNF3-10-2, BDNF3-10-4, BDNF3-10-7, BDNF3-10-10, as well as BDNF1-10-11-1, BDNF1-10-11-2, BDNF1-10-10A, BDNF1-10-10D, and BDNF4-10-5b can be prepared as generally described in Scheme 4, with the proper starting materials. In the preparation of BDNF3-10-4, 4-chlorobenzaldehyde is used in place of terephthalaldehyde and correspondingly, the Buchwald type coupling and reduction steps are not necessary.

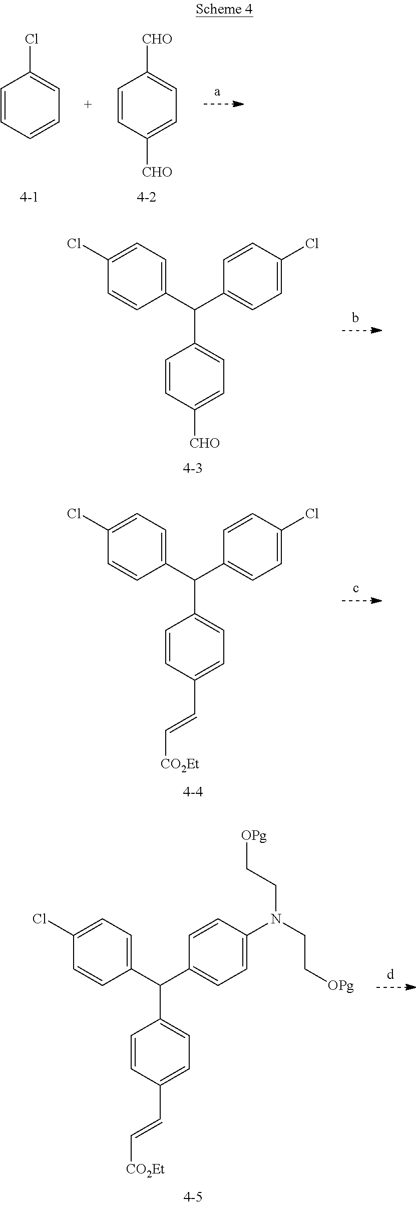

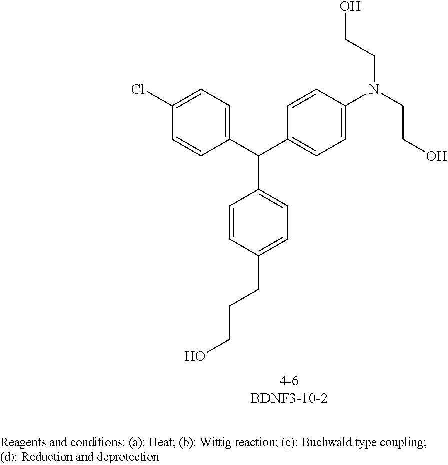 [SMILES: OCCCc1ccc(C(c2ccc(Cl)cc2)c2ccc(N(CCO)CCO)cc2)cc1]

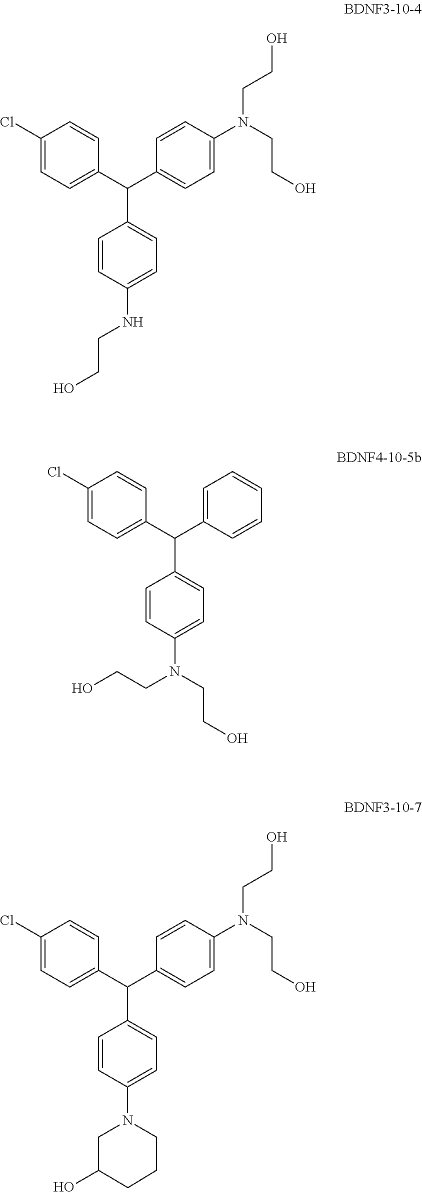 [SMILES: OCCNc1ccc(C(c2ccc(Cl)cc2)c2ccc(N(CCO)CCO)cc2)cc1 ; OCCN(CCO)c1ccc(C(c2ccccc2)c2ccc(Cl)cc2)cc1 ; OCCN(CCO)c1ccc(C(c2ccc(Cl)cc2)c2ccc(N3CCCC(O)C3)cc2)cc1]

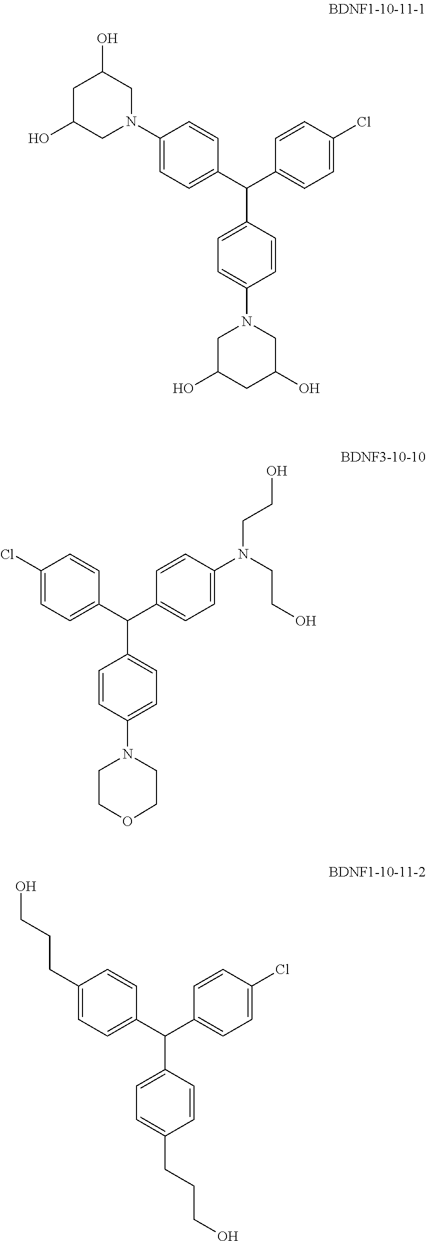 [SMILES: OC1CC(O)CN(c2ccc(C(c3ccc(Cl)cc3)c3ccc(N4CC(O)CC(O)C4)cc3)cc2)C1 ; OCCN(CCO)c1ccc(C(c2ccc(Cl)cc2)c2ccc(N3CCOCC3)cc2)cc1 ; OCCCc1ccc(C(c2ccc(Cl)cc2)c2ccc(CCCO)cc2)cc1]

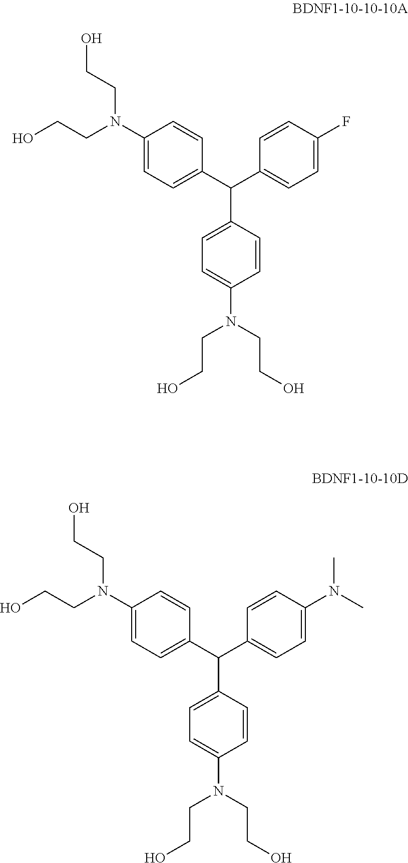 [SMILES: OCCN(CCO)c1ccc(C(c2ccc(F)cc2)c2ccc(N(CCO)CCO)cc2)cc1 ; CN(C)c1ccc(C(c2ccc(N(CCO)CCO)cc2)c2ccc(N(CCO)CCO)cc2)cc1]

Compounds BDNF2-10-1, BDNF2-10-2, BDNF2-10-3, BDNF2-10-4, BDNF3-10-5, BDNF4-10-4-a, BDNF4-10-7a and BDNF4-10-8a can be prepared in similar fashion, as generally described in Scheme 5, with the proper starting materials.

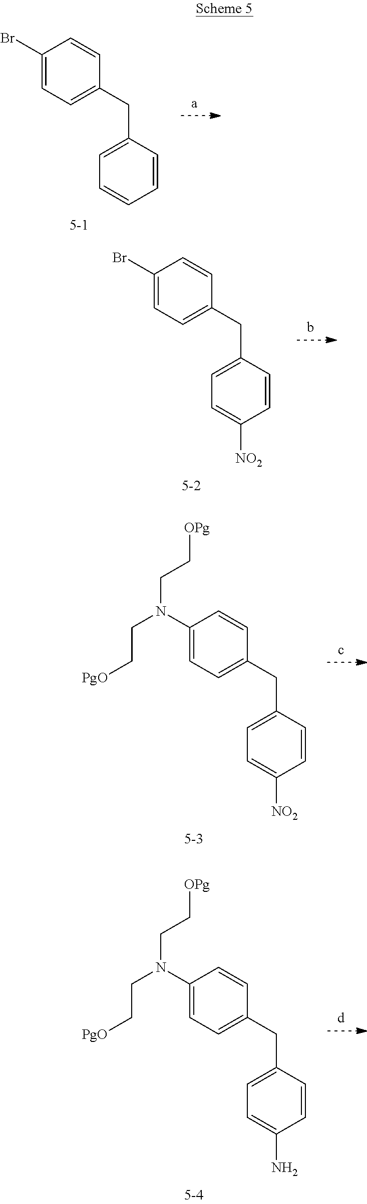

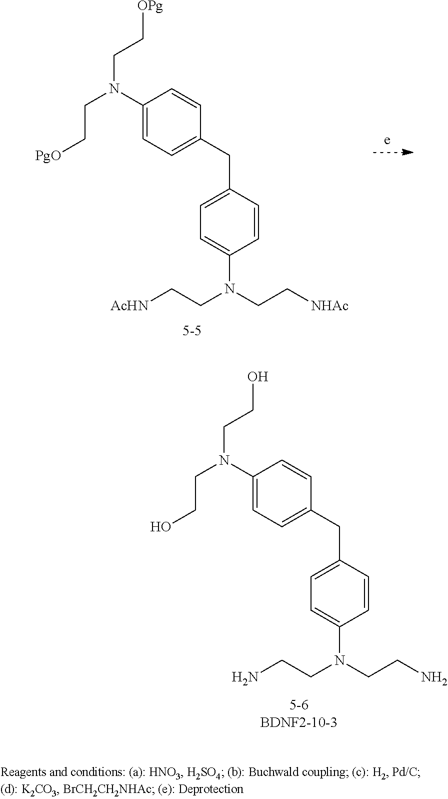

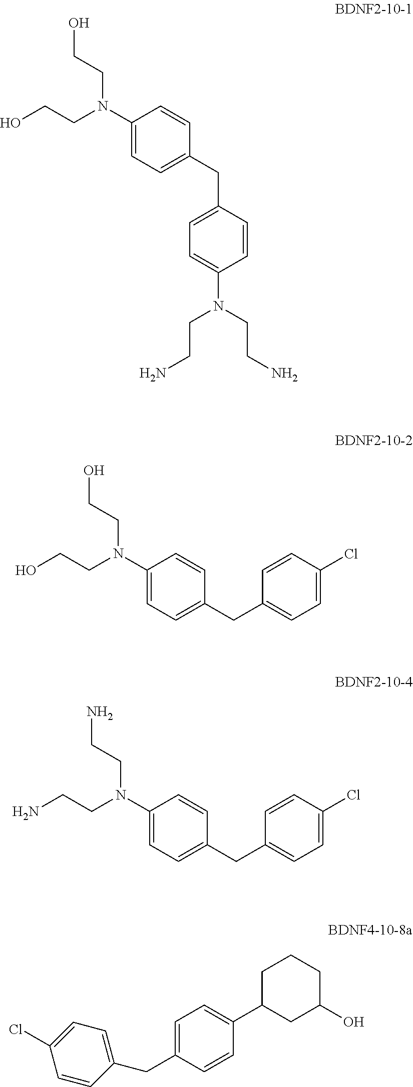 [SMILES: NCCN(CCN)c1ccc(Cc2ccc(N(CCO)CCO)cc2)cc1 ; OCCN(CCO)c1ccc(Cc2ccc(Cl)cc2)cc1 ; NCCN(CCN)c1ccc(Cc2ccc(Cl)cc2)cc1 ; OC1CCCC(c2ccc(Cc3ccc(Cl)cc3)cc2)C1]

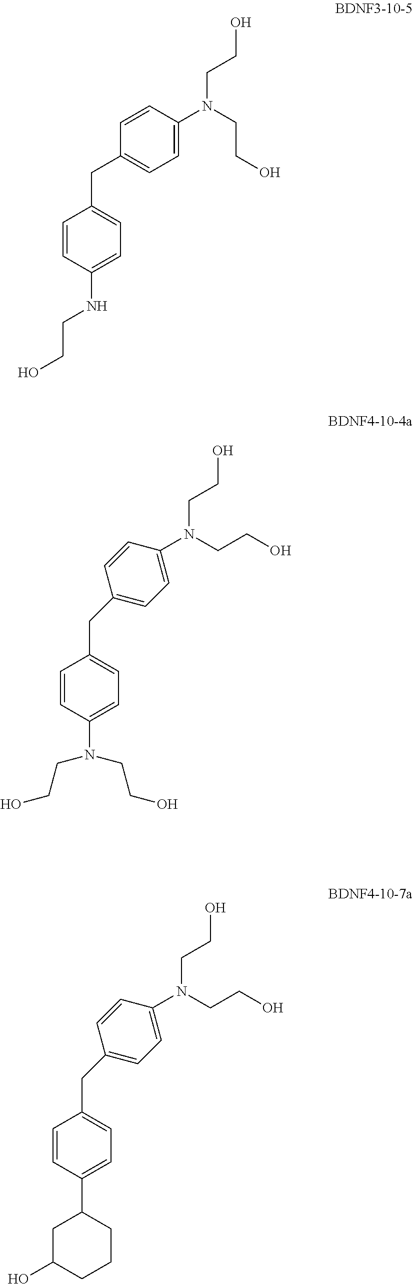 [SMILES: OCCNc1ccc(Cc2ccc(N(CCO)CCO)cc2)cc1 ; OCCN(CCO)c1ccc(Cc2ccc(N(CCO)CCO)cc2)cc1 ; OCCN(CCO)c1ccc(Cc2ccc(C3CCCC(O)C3)cc2)cc1]

Compounds BDNF3-10-12 and BDNF4-10-1a can be prepared as generally described in Scheme 6, with the proper starting materials.

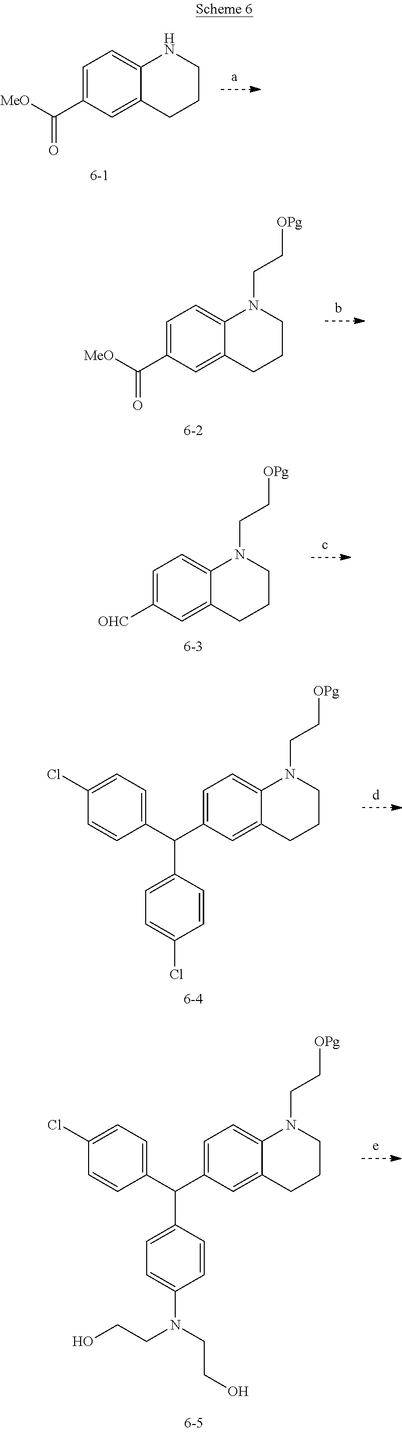

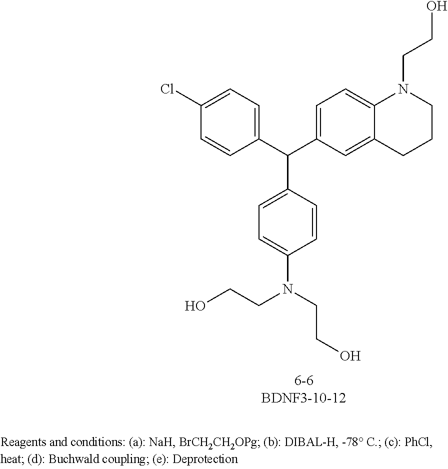 [SMILES: OCCN(CCO)c1ccc(C(c2ccc(Cl)cc2)c2ccc3c(c2)CCCN3CCO)cc1]

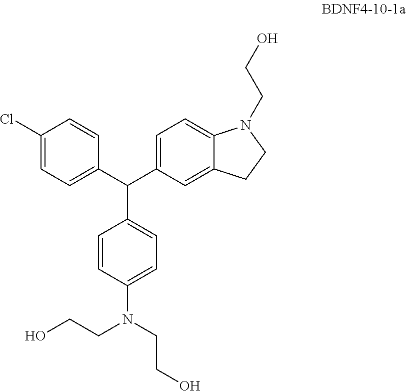 [SMILES: OCCN(CCO)c1ccc(C(c2ccc(Cl)cc2)c2ccc3c(c2)CCN3CCO)cc1]

Compound BDNF1-4-15-4 can be prepared as described in Scheme 7.

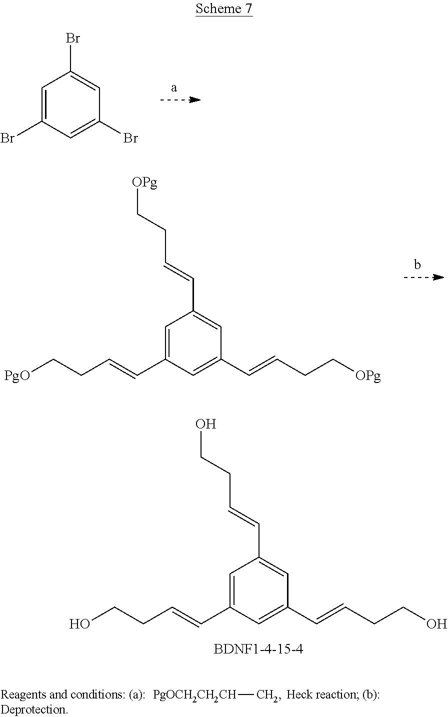

Compound BDNF1-4-15-5 can be prepared as described in Scheme 8.

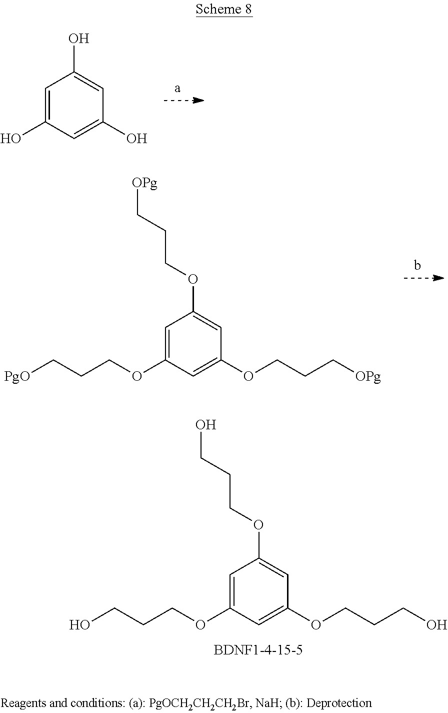

### Example

### Measurement of Activity

### Compounds Promote Hippocampal Neuron Survival

To understand the mechanisms of action of the BDNF loop 2 mimetics and to test the conjecture that they work via the targeted receptor, TrkB, the dose-dependent relationships of the survival-promoting activities of the compounds of the invention are compared to BDNF using embryonic hippocampal neurons in culture conditions in which BDNF promotes neural survival. In the cultures, neurotrophic activity is mediated by BDNF principally through TrkB and p75^{NTR}, as they express little TrkA (Brann, A. B., et al. (1999) J Neurosci 19, 8199-8206; Bui, N. T., et al. (2002) J Neurochem 81, 594-605). Dose-response profiles can provide EC₅₀ and intrinsic activities relative to the BDNF response.

### Compounds Activate TrkB Receptors

To assess the interactions of the BDNF loop 2 mimetic compounds with TrkB receptors, the ability of the compounds of the invention to activate TrkA, TrkB, and TrkC is compared in a series of Western blotting experiments. These studies can be used to show that the BDNF mimetics achieve a high level of specificity, where only TrkB is activated. NIH3T3 cells which do not express native Trk receptors were stably transfected to express human TrkA (responds to NGF), TrkB (responds to BDNF) or TrkC (responds to NT-3). Western blots were used to detect phospho-Trk which is a standard method for detecting Trk activation. Positive control studies demonstrated that each of these Trks was activated by its native ligand.

A measurement of NIH3T3 cells expressing TrkB, both BDNF and the compounds of the invention can be shown to activate TrkB based on an evaluation Trk phosphorylation (demonstrate by probing with anti-Trk^{Y490} antibodies). Culture medium alone (CM) or treatment with NGF do not activate TrkB. NGF promotes activation of TrkA, based on the resulting Trk phosphorylation, but BDNF does not.

A measurement of NIH3T3 cells expressing TrkC can also be used to evaluate the compounds of the invention. Total amount of TrkC present is determined by probing with anti-TrkC antibodies. NT-3 promotes the phosphorylation of Trk, while culture medium alone (CM) and BDNF do not.

### Compound Activity in Huntington's Disease Model

Quinolinic acid-induced death of striatal neurons is an established model for Huntington's disease (HD). Previous work has shown that BDNF functions are impaired in HD and that administration of BDNF can prevent quinolinic acid-induced death. See Perez-Navarro, E., et al. (2000) J. Neurochem 75, 2190-2199; and Kells, A. P., et al. (2004) Mol Ther 9, 682-688, each of which is herein incorporated by reference in its entirety.

In particular, measurements of % death of mouse E16 striatal neurons after application of quinolinic acid at 7.5 mM compared to culture medium alone (CM) to quinolinic acid in the absence of BDNF, over a dose range of 0 to 1.5 ng/mL, and a compound of the invention, over a dose range of 10-1000 nM, efficacies can be measured for decreasing quinolinic acid-induced death.

The K252a inhibitor is a well-characterized inhibitor of TrkB receptor activation. K252a has been shown to have no effect on quinolinic acid-induced death but blocks the ability of BDNF to prevent death. A similar study using the compounds of the invention can be used to show that the effect of the compounds of the invention is mediated through the ability to activate the TrkB receptor.

A similar study similar can be conducted in which only DARP32-positive neurons are assessed. DARP32-positive neurons represent the population within the striatum that are particularly vulnerable in Huntingdon's disease. The compounds of the invention can be shown to protect DARP32-positive neurons with an efficacy similar to that of BDNF. This protection could be mediated through TrkB; in this way the efficacy of the compounds of the invention can be compared to that of BDNF. Further, the addition of the inhibitor K252a can be shown to decrease the ability of BDNF and the compounds of the invention to prevent cell death.

### Compound Activity in Parkinson's Disease Model

Exposure of SH-SY5Y cells to 1-methyl-4-phenylpyridinium (MPP⁺) with resulting cell death is a well-characterized model of Parkinson's disease (PD). See Presgraves, S. P., et al. (2004) Exp Neurol 190, 157-170, and Dluzen, D. E., et al. (2004) Neuroscience 128, 201-208, each of which is herein incorporated by reference in its entirety. BDNF has also been previously shown to protect dopaminergic neurons. Further, MPP⁺ causes a PD-like condition in primates.

For the Parkinson's disease model assays, human SH-SY5Y cells can be used after terminal differentiation into a dopaminergic phenotype (Presgraves. S. P., et al. (2004) Exp Neruol 190, 157-170). Human SH-SY5Y neuroblastoma cells are propagated to confluence in Dulbecco's Modified Eagle's media (DMEM) supplemented with 10% fetal calf serum, 100 μg/mL penicillin, 100 μg/mL streptomycin, 0.25 μg/mL amphotericin B, and 0.01 μM non-essential amino acids and then sub-cultured for differentiation. For differentiation, the cells are incubated in same media containing 10 μM retinoic acid for 3 days; then the media was removed and replaced with media containing 160 nM of the phorbol ester 12-O-tetradecanoyl-phorbol-13-acetate (TPA) for 3 days of differentiation. The cells are then administered a range dose of a compound of the invention or BDNF (1×10⁻¹² M to 1 mM) in DMEM for 30 mins for three days prior to addition of 100 μM MPP⁺. Following transfer of MPP⁺ to the media, sister cultures are tested at 6, 12, 24, and 48 hrs for the cytotoxicity of MPP⁺ as measured by the MTT and lactate dehydrogenase method (LDH) assays which accurately measure different aspects of cell death. In addition, the neuroprotective effects of a fixed dose of the compounds of the invention and BDNF on MPP⁺ induced cell death are tested for in the presence and absence of TrkB inhibitor K252a (200 nM).

In this way, the TrkB inhibitor K252a can be shown to have a small effect on lowering cell survival of dopaminergic neurons. MPP⁺ promotes death of essentially all cells. The degree of blocking of death-inducing activity of MPP⁺ by both BDNF and compounds of the invention can be compared. It is expected that K252a blocks a significant amount of the protection afforded by BDNF and the compounds of the invention, suggesting that the protective effect is mediated through the activation of TrkB.

### Compound Activity in Alzheimer's Disease Model

The ability of the BDNF mimetics to prevent Aβ-induced neuronal degeneration can be tested as a model of the compounds' ability to treat Alzheimer's disease. E16-17 hippocampal neurons are matured for 6 days and then incubated for 3 days with the indicated combinations of Aβ oligomer preparations (Dahigren, K. N., et al. (2002) J Biol Chem 277, 32046-32053), neurotrophins and neurotrophin mimetics. Following the three day incubation, neuronal survival is assessed using standard morphological criteria (Massa, S. M., et al. (2006) J Neurosci 26, 5288-5300).

Addition of Aβ₁₋₄₂ oligomer (10 μM) but not control Aβ_{Scrambled} oligomer (10 μM) results in an approximate 40% reduction in neuronal survival. NGF fails to prevent Aβ-induced degeneration. The NGF mimetic LM11A-31, known to act as a ligand at the NGF p75^{NTR} receptor (described in published application U.S. No. 2006/0246072), blocks Aβ-induced degeneration. This blocking activity was itself blocked by NGF, consistent with NGF competing with LM11A-31 at p75^{NTR} and thereby inhibiting its protective effect. The effectiveness of BDNF mimetics of the present invention can be compared to BDNF in blocking Aβ-induced degeneration.

The patents and publications listed herein describe the general skill in the art and are hereby incorporated by reference in their entireties for all purposes and to the same extent as if each was specifically and individually indicated to be incorporated by reference. In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

1. A compound of formula (I): 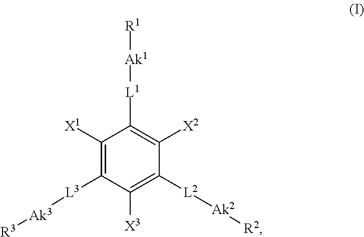
or a pharmaceutically acceptable salt, ester, prodrug, or solvate thereof;
wherein
X¹, X², and X³ each independently is —H or halo;
Ak¹, Ak², and Ak³ each independently is optionally substituted C₁-C₆ alkylene, optionally substituted C₂-C₆ alkenyl, or optionally substituted C₂-C₆ alkynyl;
L¹, L², and L³ each independently is —O—, —S—, —NH—, —C(O)NH—, —C(S)NH—, —CR^{A}R^{B}CR^{A′}R^{B′} or —CR^{A}═CR^{A′}—; or alternatively, one or two of -L¹-Ak¹-R¹, -L²-Ak²-R², and -L³-Ak³-R³ is —H;
R¹, R², and R³ each independently is —OH, —NH₂, —OR^{A}, or —NR^{A}R^{B}; and
each of R^{A}, R^{B}, R^{A′}, and R^{B′} independently is —H, halo, or C₁-C₆ alkyl;
with the proviso that the compound does not have the formula:

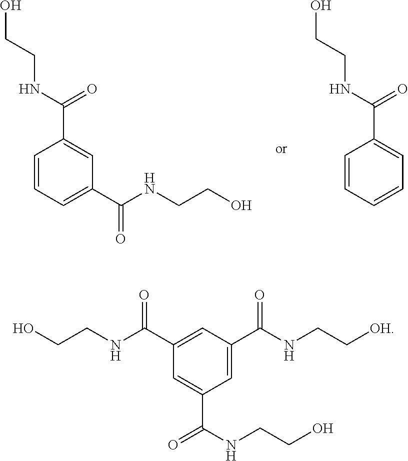 [SMILES: O=C(NCCO)c1cccc(C(=O)NCCO)c1 ; O=C(NCCO)c1ccccc1 ; O=C(NCCO)c1cc(C(=O)NCCO)cc(C(=O)NCCO)c1]

2. The compound of claim 1 wherein
each of X¹, X², and X³ is —H, —F, or —Cl;
Ak¹, Ak², and Ak³ each independently is optionally substituted C₁-C₆ alkylene; and
each of R^{A}, R^{B}, R^{A′}, and R^{B′} independently is —H, —F, or —Cl.

3. The compound of claim 2 wherein
each of X¹, X², and X³ is —H;
Ak¹, Ak², and Ak³ each independently is optionally substituted C₁-C₄ alkylene; and
R¹, R², and R³ each independently is —OH or —NH₂.

4. The compound of claim 1 wherein
each of X¹, X², and X³ is —H;
Ak¹, Ak², and Ak³ each independently is optionally substituted C₂-C₃ alkylene; and
L¹, L², and L³ each independently is —O—, —C(O)NH—, —C(S)NH—, —CH₂CH₂— or —CH═CH—; and
R¹, R², and R³ each independently is —OH or —NH₂.

5. The compound of claim 1 wherein
at least one of X¹, X², and X³ is —Cl;
Ak¹, Ak², and Ak³ each independently is optionally substituted C₁-C₄ alkylene;
R¹, R², and R³ each independently is —OH, —NH₂, —OR^{A}, or —NR^{A}R^{B}; and
each of R^{A}, R^{B}, R^{A′}, and R^{B′} each independently is —H, —F, or —Cl.

6. The compound of claim 5 wherein
at least two of X¹, X², and X³ is —Cl;
Ak¹, Ak², and Ak³ each is —CH₂—CH₂—;
L¹, L², and L³ each independently is —O—, —S—, —NH—, —C(O)NH—, —C(S)NH—, —CH₂CH₂— or —CH═CH—; and
R¹, R², and R³ each independently is —OH or —NH₂.

7. The compound according to claim 1 having a structural formula selected from the group consisting of:

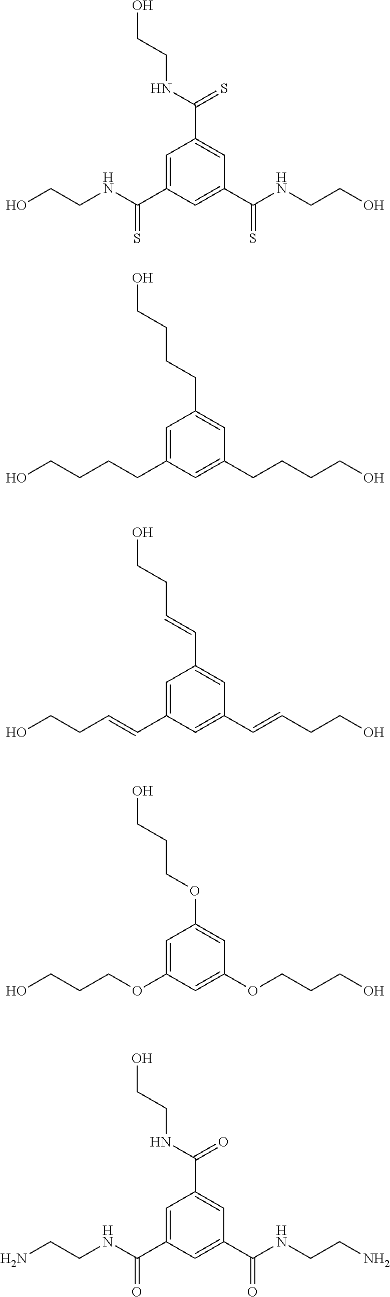

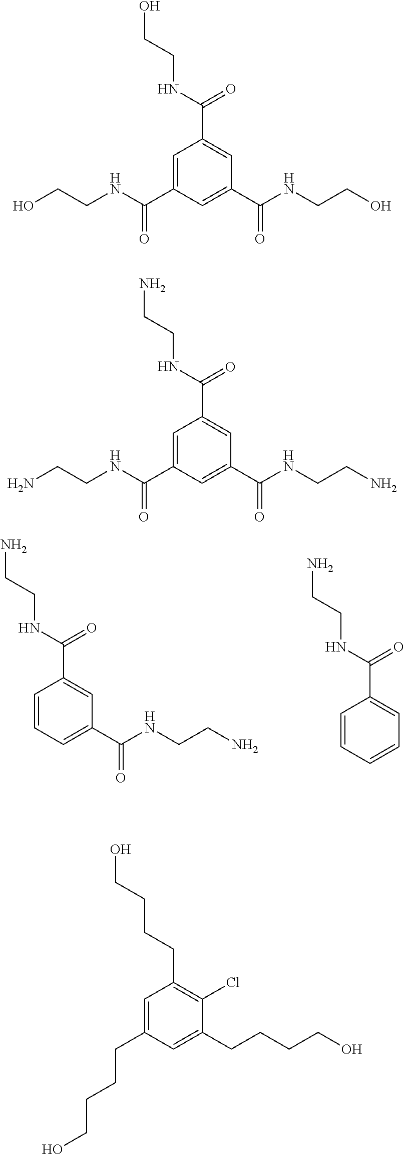

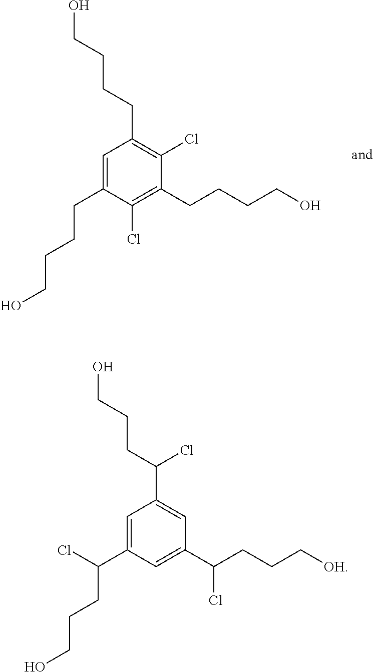 [SMILES: OCCCCc1cc(CCCCO)c(Cl)c(CCCCO)c1Cl ; OCCCC(Cl)c1cc(C(Cl)CCCO)cc(C(Cl)CCCO)c1]

8. A compound of Formula (II): 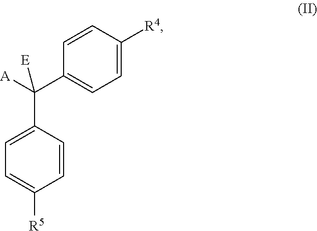
or a pharmaceutically acceptable salt, ester, prodrug, or solvate thereof;
wherein:
each of R⁴ and R⁵ is independently halo, —NR^{C}R^{D}, optionally substituted heterocycloalkyl, optionally substituted cycloalkyl, or optionally substituted aryl;
A is —H, C₁-C₆ alkyl, C₂-C₆ alkenyl, C₂-C₆ alkynyl, optionally substituted aryl, or optionally substituted heteroaryl;
E is —H or halo; and
each of R^{C} and R^{D} is independently —H, C₁-C₆ alkyl, C₁-C₆ aminoalkylene, C₂-C₆ aminoalkenyl, C₂-C₆ aminoalkynyl, C₁-C₆ hydroxyalkylene, C₂-C₆ hydroxyalkenyl, or C₂-C₆ hydroxyalkynyl;
with the proviso that the compound does not have the formula:

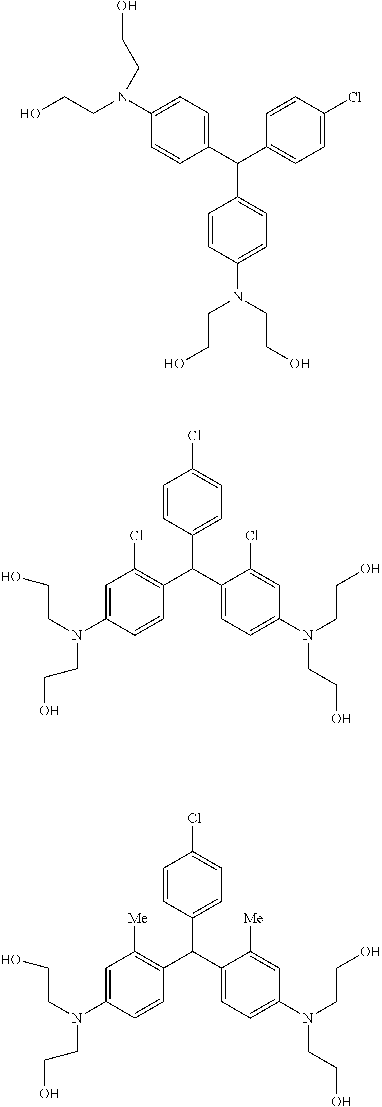 [SMILES: OCCN(CCO)c1ccc(C(c2ccc(Cl)cc2)c2ccc(N(CCO)CCO)cc2)cc1 ; OCCN(CCO)c1ccc(C(c2ccc(Cl)cc2)c2ccc(N(CCO)CCO)cc2Cl)c(Cl)c1 ; Cc1cc(N(CCO)CCO)ccc1C(c1ccc(Cl)cc1)c1ccc(N(CCO)CCO)cc1C]

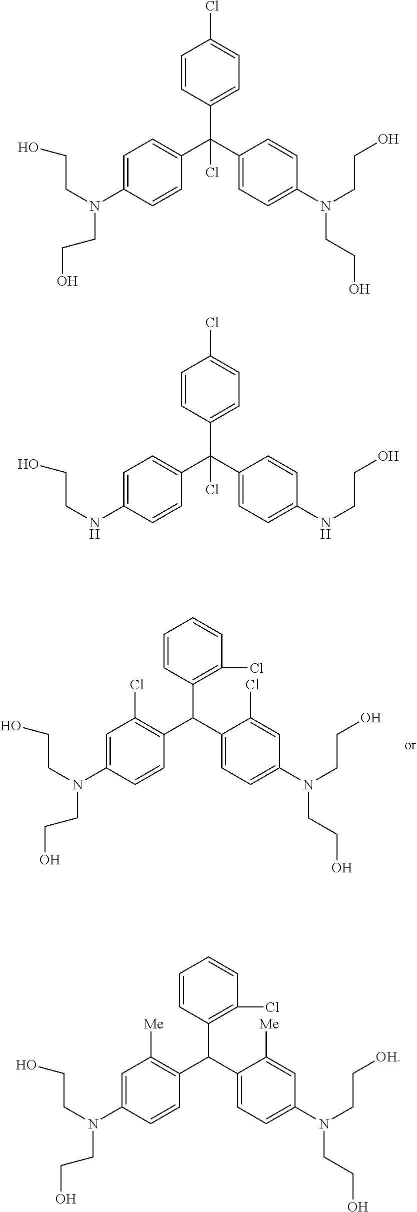 [SMILES: OCCN(CCO)c1ccc(C(Cl)(c2ccc(Cl)cc2)c2ccc(N(CCO)CCO)cc2)cc1 ; OCCNc1ccc(C(Cl)(c2ccc(Cl)cc2)c2ccc(NCCO)cc2)cc1 ; OCCN(CCO)c1ccc(C(c2ccccc2Cl)c2ccc(N(CCO)CCO)cc2Cl)c(Cl)c1 ; Cc1cc(N(CCO)CCO)ccc1C(c1ccc(N(CCO)CCO)cc1C)c1ccccc1Cl]

9. The compound of claim 8 wherein
each of R⁴ and R⁵ is independently —F, —Cl, —NR^{C}R^{D}, optionally substituted morpholinyl, optionally substituted thiomorpholinyl, optionally substituted piperazinyl, optionally substituted piperidinyl, optionally substituted pyrrolidinyl, optionally substituted cyclohexadienyl, or optionally substituted phenyl;
A is —H, C₁-C₆ alkyl, optionally substituted phenyl, or optionally substituted bicyclic heteroaryl;
E is —H or —Cl; and
each of R^{C} and R^{D} is independently —H, C₁-C₆ alkyl, C₁-C₆ aminoalkyl, or C₁-C₆ hydroxyalkyl.

10. The compound of claim 9 wherein
each of R⁴ and R⁵ is independently —F, —Cl, —NR^{C}R^{D}, optionally substituted N-bound morpholinyl, optionally substituted N-bound piperidinyl, or optionally substituted cyclohexa-1,4-dienyl;
A is —H, C₃-C₆ alkylene, optionally substituted phenyl, optionally substituted quinolinyl, or optionally substituted tetrahydroquinolinyl;
E is —H; and
each of R^{C} and R^{D} is independently —H, methyl, ethyl, C₂-C₄ aminoalkylene, or C₂-C₄ hydroxyalkylene.

11. The compound of claim 9 wherein each of A and E is —H.

12. The compound of claim 9 wherein A is C₃-C₆ alkylene and E is —H.

13. The compound of claim 9 wherein A is quinolinyl substituted with one or more of —OH and C₁-C₆ hydroxyalkylene, and E is —H.

14. The compound of claim 13 wherein A is quinolinyl substituted with C₂-C₄ hydroxyalkylene, and E is —H.

15. The compound of claim 9 wherein A is tetrahydroquinolinyl substituted with one or more of —OH and C₁-C₆ hydroxyalkylene, and E is —H.

16. The compound of claim 15 wherein A is tetrahydroquinolinyl substituted with C₂-C₄ hydroxyalkylene, and E is —H.

17. The compound of claim 9 wherein A is optionally substituted phenyl, and E is —H or —Cl.

18. The compound of claim 17 wherein A is substituted phenyl, wherein the substituent is selected from the group consisting of —Cl, -Me and —NR^{C}R^{D}, wherein each of R^{C} and R^{D} is independently —H, C₁-C₆ aminoalkylene, or C₁-C₆ hydroxyalkylene.

19. The compound of claim 18 wherein the substituent is —NR^{C}R^{D}, wherein each of R^{C} and R^{D} is C₂-C₄ aminoalkylene, or C₂-C₄ hydroxyalkylene.

20. The compound of claim 18 wherein each of R^{C} and R^{D} is —CH₂CH₂—OH.

21. The compound according to claim 10 having a structural formula selected from the group consisting of:

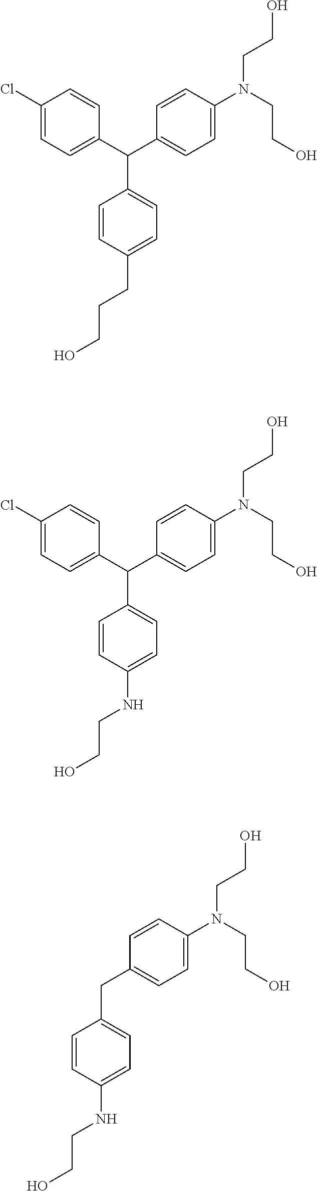 [SMILES: OCCCc1ccc(C(c2ccc(Cl)cc2)c2ccc(N(CCO)CCO)cc2)cc1 ; OCCNc1ccc(C(c2ccc(Cl)cc2)c2ccc(N(CCO)CCO)cc2)cc1 ; OCCNc1ccc(Cc2ccc(N(CCO)CCO)cc2)cc1]

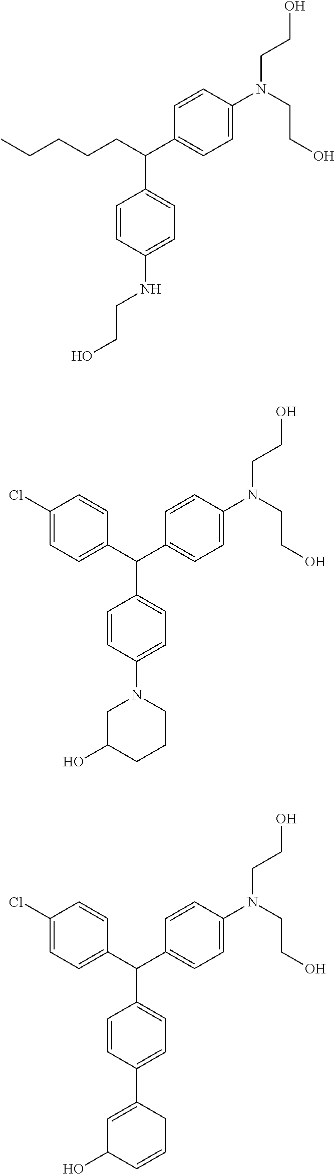 [SMILES: CCCCCC(c1ccc(NCCO)cc1)c1ccc(N(CCO)CCO)cc1 ; OCCN(CCO)c1ccc(C(c2ccc(Cl)cc2)c2ccc(N3CCCC(O)C3)cc2)cc1 ; OCCN(CCO)c1ccc(C(c2ccc(Cl)cc2)c2ccc(C3=CC(O)C=CC3)cc2)cc1]

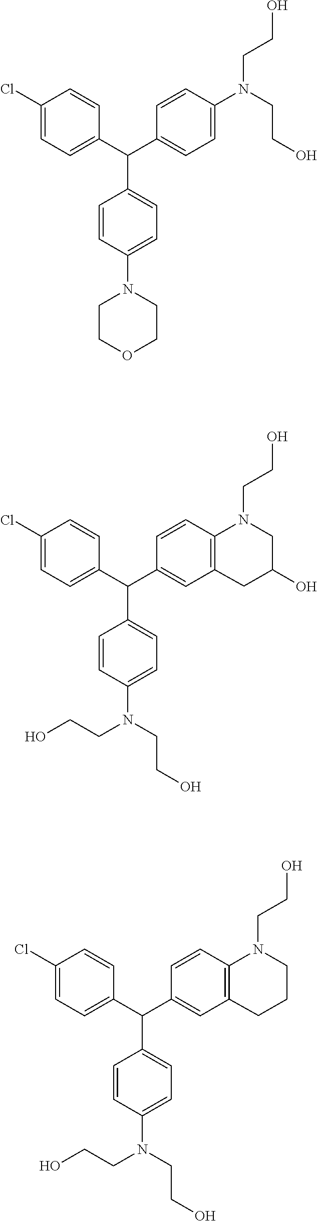 [SMILES: OCCN(CCO)c1ccc(C(c2ccc(Cl)cc2)c2ccc(N3CCOCC3)cc2)cc1 ; OCCN(CCO)c1ccc(C(c2ccc(Cl)cc2)c2ccc3c(c2)CC(O)CN3CCO)cc1 ; OCCN(CCO)c1ccc(C(c2ccc(Cl)cc2)c2ccc3c(c2)CCCN3CCO)cc1]

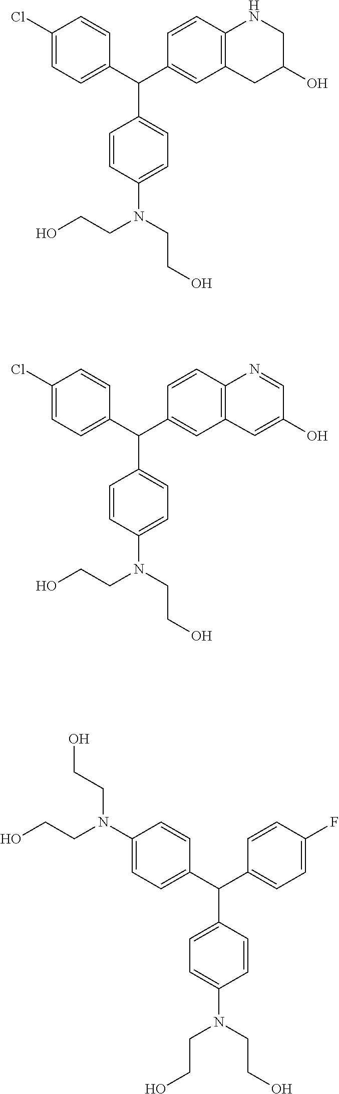 [SMILES: OCCN(CCO)c1ccc(C(c2ccc(Cl)cc2)c2ccc3c(c2)CC(O)CN3)cc1 ; OCCN(CCO)c1ccc(C(c2ccc(Cl)cc2)c2ccc3ncc(O)cc3c2)cc1 ; OCCN(CCO)c1ccc(C(c2ccc(F)cc2)c2ccc(N(CCO)CCO)cc2)cc1]

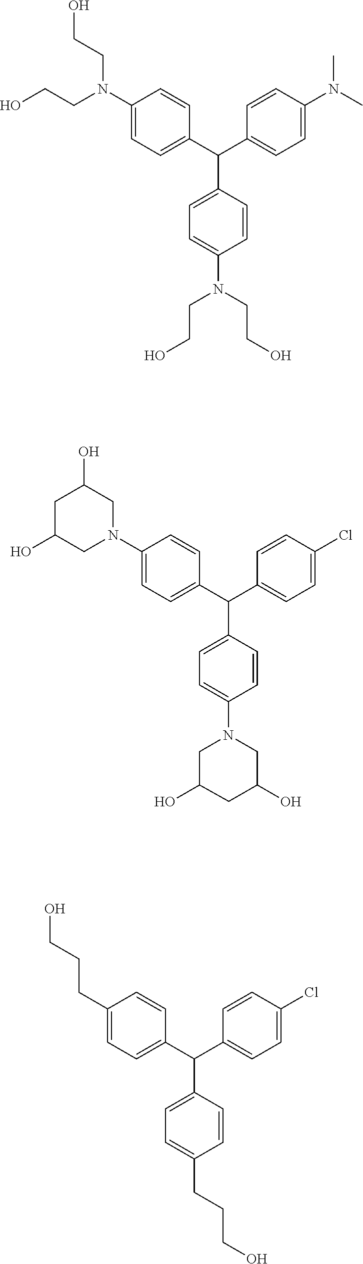 [SMILES: CN(C)c1ccc(C(c2ccc(N(CCO)CCO)cc2)c2ccc(N(CCO)CCO)cc2)cc1 ; OC1CC(O)CN(c2ccc(C(c3ccc(Cl)cc3)c3ccc(N4CC(O)CC(O)C4)cc3)cc2)C1 ; OCCCc1ccc(C(c2ccc(Cl)cc2)c2ccc(CCCO)cc2)cc1]

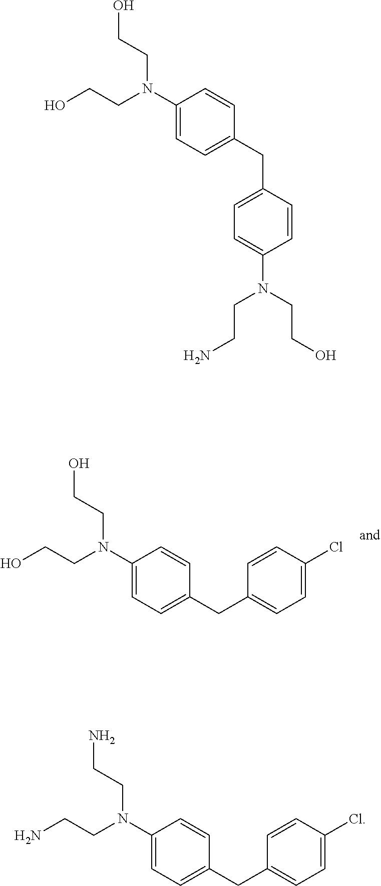 [SMILES: NCCN(CCO)c1ccc(Cc2ccc(N(CCO)CCO)cc2)cc1 ; OCCN(CCO)c1ccc(Cc2ccc(Cl)cc2)cc1 ; NCCN(CCN)c1ccc(Cc2ccc(Cl)cc2)cc1]

22-23. (canceled)

24. A method of treating a disorder that can be treated by contacting, activating or inhibiting a TrkB receptor in a subject in need of treatment thereof, comprising administering to the subject an effective amount of a compound of claim 1.

25. The method of claim 24, wherein the disorder is selected from the group consisting of Alzheimer's disease, Huntington's disease, amyotrophic lateral sclerosis, Rett syndrome, epilepsy, Parkinson's disease, spinal cord injury, stroke, hypoxia, ischemia, brain injury, diabetic neuropathy, peripheral neuropathy, nerve transplantation complications, motor neuron disease, multiple sclerosis, HIV dementia, peripheral nerve injury, hearing loss, depression, obesity, metabolic syndrome, pain, cancer, and other conditions involving degeneration or dysfunction of cells expressing TrkB.

26-28. (canceled)

29. A method of facilitating cell survival comprising treating a TrkB-expressing cell with a compound of claim 1.

30. (canceled)

31. The method of claim 29, wherein said TrkB-expressing cell is a neuronal cell.

32-33. (canceled)

34. A method for activating a TrkB receptor molecule comprising contacting a cell containing a TrkB receptor molecule with an effective amount of a compound of claim 1.

35. (canceled)

36. A pharmaceutical formulation comprising a compound of claim 1 and a pharmaceutical grade carrier.

37-39. (canceled)
